(19) United States
(12) Patent Application Publication
(10) Pub. No.: US 2025/0010067 A1
(43) Pub. Date: Jan. 9, 2025

(54) RESTRICTION DEVICE

(71) Applicant: IMPLANTICA PATENT LTD., Lund (SE)

(72) Inventor: Peter Forsell, Bouveret (CH)

(21) Appl. No.: 18/687,891

(22) PCT Filed: Aug. 26, 2022
(86) PCT No.: PCT/EP2022/073860

§ 371 Date: Feb. 29, 2024

(30) Foreign Application Priority Data
Aug. 30, 2021 (WO) PCT/EP2021/073893
Feb. 18, 2022 (SE) 2250189-4

(51) Int. Cl. A61N 1/36; A61N 1/05; A61N 1/372; A61N 1/378

(52) U.S. Cl. CPC A61N 1/36007; A61N 1/0509; A61N 1/3615; A61N 1/36178; A61N 1/37217; A61N 1/37252; A61N 1/3787

(57) ABSTRACT

The present disclosure relates to a system for treating a patient having a disorder related to a patient's intestine (100), comprising one or more electrical stimulation devices (10) having one or more electrodes (11) for electrically stimulating muscle or neural tissue of the intestine, wherein each of the one or more electrical stimulation devices (10) comprises a wireless energy receiver (R) configured to receive energy for stimulating the muscle or neural tissue wirelessly.

### TECHNICAL FIELD

The present invention relates to a system for treating a patient having a disorder related to a patient's intestine. More specifically, the treatment involves electrical stimulation of the patient's intestine. Also disclosed is a method of implantation and a method of use of such system.

### BACKGROUND

Intestinal disorders may be caused by injury, birth defect, cancer or other diseases, such as constipation or incontinence. WO 2011/128124 A1 discloses a system for regulating the flow of intestinal contents through the intestine. In that particular application, a reservoir for intestinal contents is formed from surgically modified intestine that has been cut along a mutual contact line of laterally adjacent sections of a bent portion of intestine and connected so that the resulting upper and lower halves of the intestine form an intestinal wall of the reservoir, and the system is designed for emptying such intestinal reservoir. More specifically, the prior art system comprises a pump adapted to act on said intestinal wall so as to reduce the reservoir's volume, thereby emptying the reservoir. The pump may be an electrical stimulation type pump, a hydraulically acting type pump or/and a mechanically acting type pump. The system further comprises an entry valve upstream of the reservoir and an exit valve downstream of the reservoir.

The electrical stimulation type pump comprises an electrical stimulation device for electrically stimulating a muscle or neural tissue of said intestinal wall by applying electrical pulses to the intestinal wall so as to cause at least partial contraction thereof, in particular by a series of electrical pulses. For this purpose, the electrical stimulation apparatus comprises one or more electrodes adapted to generate the electrical pulses. This is a very gentle way of constricting the reservoir. By electrically stimulating different portions of said intestinal wall in a direction of natural intestinal contents flow over time, the intestinal contents are pumped along the intestinal reservoir and, thus, the intestinal reservoir is emptied. More specifically, the electrodes of the electrical stimulation type pump are mounted on one or more holding devices which are in the form of a cable or have any other longitudinal, stripe-like or rod-like or plate-like shape. A plurality of the electrodes may be arranged in one or more rows along the length of the holding devices. The longitudinal holding devices are arranged side by side, when implanted, so as to cover substantially the entire intestinal reservoir on one side or on opposing sides of the reservoir.

The holding devices are either embedded in a flexible web which allows the holding devices to follow movements of the intestinal reservoir when sections thereof are constricted individually due to selective electrical stimulation. Or the longitudinal holding devices are implanted in surgically created folds of the intestinal wall of the reservoir. Alternatively, the electrodes are directly invaginated in the intestinal wall one by one or in groups without being carried on a common holding device. It is further suggested in WO 2011/128124 A1 that, instead of providing a plurality of longitudinal holding devices with electrodes, the electrical stimulation device may be formed as an integral unit on at least one side of the reservoir to make handling and manufacture easier.

In addition to the electrical stimulation type pump, the pump disclosed in WO 2011/128124 A1 may comprise a constriction type pump implanted in the patient's body for at least partly constricting the intestinal reservoir mechanically or hydraulically by acting from outside on the intestinal wall. Therein, the electrical stimulation type pump and the constriction type pump may act on the same portions of the intestinal wall so as to pump the intestinal contents along the reservoir by, over time, electrically stimulating different portions of said intestinal wall and simultaneously constricting respective sections of the reservoir in the direction of natural intestinal contents flow. In particular, the constriction type pump in operation may constrict the intestinal reservoir only partly, in order not to damage the intestinal tissue, whereas complete constriction and, thus, emptying of the reservoir is obtained by additionally stimulating the intestinal wall portions electrically in a manner as described before.

Since due to the surgical modifications, the intestinal reservoir itself has lost its natural peristaltic capabilities, the electrical stimulation type pump may pump intestinal contents along the reservoir in a direction of natural intestinal contents flow by stimulating different portions of the intestinal wall in a wavelike (peristaltic) manner, e.g. when constriction of the reservoir caused by the constriction type pump is released, so as to improve the filling of the intestinal reservoir with intestinal contents supplied to the reservoir. An exit valve provided at the downstream end of the intestinal reservoir is closed while the reservoir is filling up, to prevent intestinal contents from escaping the reservoir unintentionally.

It is an object of the present invention to further improve the system for treating a patient having a disorder related to a patient's intestine which involves electrical stimulation of the patient's intestine. In this regard, the system may comprise all the features as described above in relation to WO 2011/128124 A1, but with some modifications as described hereinafter.

### SUMMARY

According to one aspect of the present disclosure, the system for treating a patient having a disorder related to a patient's intestine comprises a plurality of electrical stimulation devices having one or more electrodes for electrically stimulating muscle or neural tissue of the intestine, wherein each of the one or more electrical stimulation devices comprises a wireless energy receiver configured to receive energy for stimulating the muscle or neural tissue wirelessly.

This means that the electrical stimulation devices are not connected by wire, nor in any other way. Physically, the electrical stimulation devices are independent from each other. This way, they can be installed on or close to the intestine or even implanted in an intestinal wall and are able to follow any movement of the intestine. In particular, such intestinal movement may be caused by electrical stimulation via the respective electrical stimulation device and/or by constriction via the aforementioned constriction type pump and/or by constriction via any other mechanical or hydraulic or other type of constriction device. In other words, the one or more electrical stimulation devices are rather flexible and remain flexible over time since any danger that such flexibility may decrease due to fibrosis growing over and encapsulating the system and electrical stimulation devices is minimized, thanks to the physical independence of the electrical stimulation devices.

The system preferably comprises one or a plurality of wireless energy transmitters configured to transfer energy to some or all of the one or more electrical stimulation devices. Thus, one energy transmitter is provided for supplying energy to more than one electrical stimulation device. More specifically, either one wireless energy transmitter may be configured to transfer energy to all of the one or more electrical stimulation devices or at least one, preferably all, of the plurality of wireless energy transmitters may be configured to transfer energy to some of the one or more electrical stimulation devices. This way, the complexity of the system may be kept minimal. Of course, more than one energy transmitter may be provided, each one of them configured to supply energy to more than one electrical stimulation device.

The wireless energy receiver of each of the one or more electrical stimulation devices may include a secondary coil and at least one of the wireless energy transmitters, preferably each transmitter, may comprise a primary coil configured to induce a voltage in the secondary coil of some or all of the one or more electrical stimulation devices. This way, energy can be transmitted wirelessly from the energy transmitter to the energy receiver via the primary and secondary coils.

Alternatively, the system may comprise an individual wireless energy transmitter for each one of the one or more electrical stimulation devices for transferring energy individually to the respective one of the one or more electrical stimulation devices. In this case, in order to transmit energy wirelessly, the wireless energy receiver of each of the one or more electrical stimulation devices may include a secondary coil and each of the individual wireless energy transmitters may comprise a primary coil configured to transfer energy to the secondary coil of a respective one of the one or more electrical stimulation devices.

Preferably, RFID technology is used to transfer the energy wirelessly from the energy transmitter to the energy receiver. RFID technology is widely known, and transfer of energy via the aforementioned primary and secondary coils is a well-known way of transferring energy by RFID technology. More specifically, the wireless energy receiver may be configured to receive the energy via RFID pulses.

The system preferably comprises a feedback unit configured to provide feedback pertaining to the amount of energy received by the wireless energy receiver, such as via the RFID pulses, wherein the system is configured to adjust the amount of transferred energy based on the feedback. More specifically, the amount of RFID pulse energy that is being received may be adjusted based on the feedback such that the pulse frequency is successively raised until a satisfying level is reached.

Preferably, each of the one or more electrical stimulation devices comprises a rechargeable energy storage unit, such as a rechargeable battery or a capacitor, for temporarily storing at least part of the wirelessly received energy. The rechargeable energy storage unit may be charged over time so that an energy amount required by the electrode or electrodes of the respective electrical stimulation device for stimulating the muscle or neural tissue is available when needed. Such energy amount may be small anyways, as contraction of the muscle is autonomous once an activation potential in the corresponding nerve has been reached or exceeded by means of the electrical stimulation.

Preferably, each of the one or more electrical stimulation devices comprises an internal controller. The internal controller may serve various functions, the main function consisting in controlling the timing and amount of energy applied to the electrode or electrodes of the electrical stimulation device for stimulating the nerve or muscle tissue. Another important function consists in controlling and possibly storing away the amount of energy that is received via the wireless energy receiver. The internal controller may further serve to communicate with an external controller and/or with a remote controller. For instance, such communication may relate, inter alia, to the energy transfer via the energy receiver and/or to the timing and/or amount of energy to be applied to the electrode or electrodes.

In particular, the internal controller may be configured to wirelessly receive electrode control data for controlling stimulation of the muscle or neural tissue. Thus, not only the energy transfer but also data transfer is carried out wirelessly in order for the electrical stimulation devices to be physically independent from each other. Such data may be received either from an implanted external controller or from a remote controller outside the patient's body.

Preferably, the internal controller receives the electrode control data wirelessly via the wireless energy receiver. In other words, the same port may be used to receive both energy and data. In particular, the energy transferred to and received by the electrical stimulation device via the wireless energy receiver may be appropriately modulated, the modulation defining and, thus, carrying a signal which may be decoded by the internal controller and interpreted as data. This is a well-known technique, which is particularly known and used within the RFID technology. That is, an RFID signal may be used to transport both energy and information.

More specifically, the internal controller of each of the one or more electrical stimulation devices may be addressable individually by an external controller or remote controller using an individual code, i.e. a code which is specific to the respective internal controller. This is particularly useful where one external controller or remote controller is used to control more than one electrical stimulation device and/or where one wireless transmitter is used to transmit energy wirelessly to the wireless energy receivers of more than one electrical stimulation device. For instance, when electrical stimulation devices are to be activated sequentially, e.g. for stimulating the intestine in a wave-like manner, the respective electrical stimulation device may be addressed individually using the individual code of the corresponding internal controller. Typically, such individual code is placed at the beginning of the data transmitted to the internal controller. This way, only one or more desired electrical stimulation devices may be instructed at a given time to electrically stimulate a portion of the intestine and/or only one or more desired electrical stimulation devices will receive and possibly store energy received through the wireless energy receiver.

As mentioned before, the system may comprise an external controller configured to communicate with the internal controller wirelessly. The external controller is either an implantable external controller configured to be implanted within the patient's body or a remote controller configured to communicate directly with the internal controller from outside the patient's body. Alternatively, the system may comprise a remote controller configured to communicate with the implantable external controller from outside the patient's body. In the latter case, there are at least three types of controllers, the internal controller within each one of the electrical stimulation devices, at least one external controller inside the patient's body for communication with one or more of the internal controllers, and preferably only one remote controller outside the patient's body for communicating with the one or more implanted external controllers. The remote controller is preferably operable by the patient and/or a caretaker.

The remote controller may be configured to communicate with the implantable external controller via electric wiring. However, preferably, the remote controller is configured to communicate with the implantable external controller wirelessly, which is more convenient for the patient and/or care person. Energy transfer and/or data transfer between the remote controller and the implantable external controller may be realized in the same way as the energy and/or data transfer to (and from) the internal controller of the electrical stimulation devices. In any case, the remote controller is preferably configured so that it can be mounted to the patient's skin.

The system may comprise a large number of the electrical stimulation devices. These may be arranged at many different locations along the part of the intestine which is to be stimulated electrically, i.e. in one or more rows and/or on one or two or more than two sides of the intestine, in particular on two opposite sides thereof. For instance, 4 or 5 or 6 or 7 or 8 or 9 or 10 or 11 or 12 or more than 12 of the electrical stimulation devices may be provided. Each of the electrical stimulation devices may comprise a single electrode or a plurality of two or more electrodes.

In one embodiment of the present disclosure, the system is configured to electrically stimulate, by means of the electrodes of the one or more electrical stimulation devices, the muscle or neural tissue sufficiently for a muscle of the intestine to contract to an extent such that the intestine constricts. That is, the system may function as a constriction device by electrically stimulating contraction of the muscles in the intestine.

In this context, the one or more electrical stimulation devices may form part of an electrical stimulation type pump as described above in relation to the prior art disclosed in WO 2011/128124 A1, whose pump is configured to advance intestinal contents through the patient's intestine in a downstream direction, e.g. by successive electrical stimulation of different portions of the intestine in a wavelike, i.e. peristaltic, manner.

More specifically, in the same way as the system disclosed in WO 2011/128124 A1, the system described herein may be configured and is particularly suitable for use on a reservoir section of the intestine which is formed from surgically modified intestine that has been cut along a mutual contact line of laterally adjacent sections of a bent portion of intestine and connected so that the upper and lower halves of the cut intestine form an intestinal wall of the reservoir section. More specifically, at least the electrodes of the one or more electrical stimulation devices may be configured to be implanted in surgically created folds of the patient's intestine.

In addition to the one or more electrical stimulation devices, the system may further comprise at least one mechanical or hydraulic constriction device configured to be implanted outside the patient's intestine in close proximity thereto for constricting the intestine from the outside thereof. The electrical stimulation devices and the mechanical or hydraulic constriction device may be configured to act on the same part of the patient's intestine, as is generally known from WO 2011/128124 A1. In this context, the mechanical or hydraulic constriction device may form part of a pump that is configured to advance intestinal contents through the patient's intestine in a downstream direction. Alternatively, the mechanical or hydraulic constriction device may have the function of a valve configured to open and close the intestine by constriction to thereby control the flow of intestinal contents through the intestine, in particular into or out of the intestine. For instance, the valve may form an artificial sphincter close to the patient's rectum or close to a stoma of the patient. The electrical stimulation devices may support the respective function of the mechanical or hydraulic constriction device. They may individually or together form an emptying device for emptying a respective section of the patient's intestine.

In another embodiment of the present disclosure, the system is configured to electrically stimulate, by means of the electrodes of the one or more electrical stimulation devices, the muscle or neural tissue in an area of the intestine constricted by a medical device, such as the at least one mechanical or hydraulic constriction device sufficiently for increasing blood flow through the tissue of the intestine. The purpose thereof is to exercise the tissue wall which is in contact with the constriction device. The body tends to react to medical implants, partly because the implant is a foreign object, and partly because the implant interacts mechanically with tissue of the body. Exposing tissue to long-term engagement with, or pressure from, a mechanical or hydraulic or other type of constriction device may deprive the tissue cells of oxygen and nutrients, which may lead to deterioration of the tissue, atrophy and eventually necrosis. This may result in migration of the device, including migration through the tissue wall. It is therefore desirable to exercise the tissue cells so as to stimulate blood flow and increase tolerance of the tissue for pressure from the implant. In this context, it is preferable to configure the system such that electrical stimulation of the muscle or neural tissue for increasing blood flow through the tissue of the intestine is adjustable at a low level which is not enough to constrict the intestine.

### Communication

According to a further aspect of the present disclosure, security of the system against fraudulent third-party intervention may be increased. This is particularly important in the context of wireless communication, which can easily be intercepted and then misused by third parties.

Accordingly, the system is preferably configured such that at least one of:
wireless communication from or to, or both from and to, a controller of the system is encrypted,
data transmitted by a controller via wireless communication is signed, and
authentication of a user of the system involves input of authentication data of the patient.

Preferably, the encrypted wireless communication includes encryption with a public key and decryption with a private key, such as the well-known RSA encryption. Other encryption methods may likewise be implemented. Preferably, the security level is further increased in that the private key may be a combined key derived by combining at least a first key and a second key.

Similarly, as regards the signing of the data transmitted wirelessly by a controller, such as by the aforementioned external controller or remote controller to the internal controller, the signing may involve a private key, whereas subsequent verification of the signed data may involve a corresponding public key.

Preferably, data communication involves both an encryption and a signature. The RSA encryption technology allows for both, encrypting the data and adding a digital signature to the data. For the encryption/decryption process, the sender uses a public key of the recipient for encrypting the data and the recipient uses his private key for subsequently decrypting the data, whereas for the signing/authentication process, the sender uses his private key to sign the (encrypted) data and the recipient uses the sender's public key to authenticate the signature.

As regards the authentication of a user which involves input of authentication data of the patient, the system may comprise a verification unit which is configured to obtain the authentication data of the patient. For instance, the verification unit may comprise at least one of a fingerprint reader, a retina scanner, a camera, a graphical user interface for inputting a code, and a microphone. Only after a positive verification by the verification unit will certain functions of the system be enabled. For instance, the positive verification may enable the controller to process certain data or may open a communication channel between two controllers of the system, such as a wireless communication channel.

Alternatively or in addition, the system may comprise a sensation generator for generating a sensation which is detectable by a sense of the patient. In this course, the patient may input into the system authentication data which relate to what the patient has sensed. Then, the authentication of the user may involve a verification by the verification unit that the authentication data input by the user matches data from the sensation generator which relate to the sensation generated by the sensation generator. Again, only after a positive verification by the verification unit will certain functions of the system be enabled. For instance, the positive verification may enable the controller to process certain data or may open a communication channel between two controllers of the system, such as a wireless communication channel.

In this context, the sensation generator may be configured to generate as the sensation detectable by the sense of the patient at least one of:
a vibration, which may include e.g. a fixed-frequency mechanical vibration,
a sound, which may include e.g. a superposition of fixed-frequency mechanical vibrations,
a photonic signal, which may include e.g. a non-visible light pulse, such as an infrared pulse,
a light signal, which may include e.g. a visual light pulse,
an electrical signal, which may include e.g. an electrical current pulse, and
a heat signal, which may include e.g. a thermal pulse.

The electrodes may comprise a bare electrode portion configured to form a metal-tissue interface with the tissue of the intestinal wall, thereby allowing faradaic charge transfer to be the predominant charge transfer mechanism over said interface.

Alternatively, the electrodes may comprise an electrode portion at least partly covered by a dielectric material configured to form a dielectric-tissue interface with the tissue of the intestinal wall, thereby allowing for a faradaic portion of the charge transfer mechanism over said interface to be reduced.

The arrangement of the electrical stimulation devices may be configured such that at least two electrodes can be arranged on opposing sides of the patient's intestine.

### General Communication Housing

Further, an external device configured for the communication with the implantable medical device when implanted in a patient is provided, the external device comprising: a display device and a housing unit configured to mechanically and disconnectably connect to the display device, wherein the housing comprises a first communication unit for receiving communication from the display device and a second communication unit for wirelessly transmitting communication to the implantable medical device.

According to one embodiment, the external device comprises a handheld electronic device.

According to one embodiment, the external device is configured for communicating with the implantable medical device for changing the operational state of an implantable medical device. The advantage of the embodiment is that the operational state of the implantable medical device can be changed remotely.

According to one embodiment, the first communication unit is a wireless communication unit for wireless communication with the display device. The advantage of the embodiment is that the display device can be communicated without the need of electric wires.

According to one embodiment, the first communication unit is configured to communicate wirelessly with the display device using a first communication frequency and the second communication unit is configured to communicate wirelessly with the implantable medical device using a second communication frequency, wherein the first and second communication frequencies are different. The advantage of the embodiment is that the likelihood of interferences is reduced.

According to one embodiment, the second communication unit is configured to communicate wirelessly with the implantable medical device using electromagnetic waves at a frequency below 100 kHz.

According to one embodiment, the second communication unit is configured to communicate wirelessly with the implantable medical device using electromagnetic waves at a frequency below 40 kHz. The advantage of the embodiment is that titanium, which is commonly used for medical devices, is transparent for electromagnetic waves below 40 kHz.

According to one embodiment, the first communication unit is configured to communicate wirelessly with the display device using electromagnetic waves at a frequency above 100 kHz. The advantage of the embodiment is that the frequency spectrum below 100 kHz remains noise free for the communication with the medical implantable device.

According to one embodiment, the first communication unit is configured to communicate wirelessly with the display device using a first communication protocol and the second communication unit is configured to communicate wirelessly with the implantable medical device using a second communication protocol, wherein the first and second communication protocols are different. The advantage of the embodiment is that the protocol can be independently chosen for the communication of the first and second communication units, depending on which protocol suits the needs of the communication units better.

According to one embodiment, the housing unit comprises a first antenna configured for wireless communication with the display device and a second antenna configured for wireless communication with the implantable medical device. The advantage of the embodiment is that the antenna can be independently chosen for the communication of the first and second communication units, depending on which antenna suits the needs of the communication units better.

According to one embodiment, the first communication unit is a wire-based communication unit for wire-based communication with the display device. The advantage of the embodiment is that the communication of the first communication unit is reliable and secure.

According to one embodiment, the display device comprises a first communication unit for communication with the housing unit and a second communication unit for wireless communication with a second external device. The advantage of the embodiment is that communication with an additional external device becomes possible, thereby introducing redundancy and reliability.

According to one embodiment, the second communication unit of the display device is configured for communicating with the second external device over the internet. The advantage of the embodiment is that the display device can communicate with devices far away.

According to one embodiment, the first communication unit of the display device is a wireless communication unit for wireless communication with the housing unit. The advantage of the embodiment is that the communication unit can be connected to the housing unit without the use of wires.

According to one embodiment, the first communication unit of the display device is configured to communicate wirelessly with the housing unit using a first communication frequency and the second communication unit of the display device is configured to communicate wirelessly with the second external device using a second communication frequency, wherein the first and second communication frequencies are different. The advantage of the embodiment is that the likelihood of interferences is reduced and the signal to interference and noise ratio is increased.

According to one embodiment, the first communication unit of the display device is configured to communicate wirelessly with the housing unit using a first communication protocol and the second communication unit of the display device is configured to communicate wirelessly with the second external device using a second communication protocol, wherein the first and second communication protocols are different. The advantage of the embodiment is that the protocol can be independently chosen for the communication of the first and second communication units, depending on which protocol suits the needs of the communication units better.

According to one embodiment, the display device comprises a first antenna configured for wireless communication with the housing and a second antenna configured for wireless communication with the second external device. The advantage of the embodiment is that the antenna can be independently chosen for the communication of the first and second communication units, depending on which antenna suits the needs of the communication units better.

According to one embodiment, the first communication unit is a wire-based communication unit for wire-based communication with the housing unit. The advantage of the embodiment is that the communication of the first communication unit is reliable and secure.

According to one embodiment, the display device is configured to display a user interface to the patient. The advantage of the embodiment is that the patient can use his familiar display device to communicate with the housing unit.

According to one embodiment, the housing unit is configured to transmit information pertaining to the display of the user interface to the display device. The advantage of the embodiment is that the patient can receive information using his familiar display device.

According to one embodiment, the display device is configured to receive from the patient input pertaining to communication to or from the implantable medical device and transmit signals based on the received input to the housing unit. The advantage of the embodiment is that the patient can use his familiar display device to communicate with the housing unit.

According to one embodiment, the display device comprises a touch screen configured to display the user interface and receive the input from the patient. The advantage of the embodiment is that the patient can use a familiar way of handling the information.

According to one embodiment, the housing unit is configured to display a user interface to the patient. The advantage of the embodiment is that the housing unit can receive user input.

According to one embodiment, the first communication unit of the housing unit is configured to receive communication from the implantable medical device pertaining to input from the patient and wirelessly transmit signals based on the received input to the implantable medical device, using the second communication unit. The advantage of the embodiment is that the housing unit acts as an extra node in the communication between the display device and the medical implantable device, thereby enabling it to monitor the communication.

According to one embodiment, the second communication unit of the housing unit is configured for wireless communication with the implantable medical device using a standard network protocol. The advantage of the embodiment is that the implementation of the communication units is cheap and the protocols are reliable.

According to one embodiment, the standard network protocol is one of the list of: Radio Frequency type protocol, RFID-type protocol, WLAN-type protocol, Bluetooth-type protocol, BLE-type protocol, NFC-type protocol, 3G/4G/5G-type protocol, and GSM-type protocol.

According to one embodiment, the second communication unit of the housing unit comprises a Bluetooth transceiver.

According to one embodiment, the second communication unit of the housing unit is configured for wireless communication with the implantable medical device using a proprietary network protocol. The advantage of the embodiment is that the housing unit is compatible with implantable medical devices that use proprietary network protocols.

According to one embodiment, the second communication unit of the housing unit comprises a UWB transceiver. The advantage is that high data rates can be communicated via the second communication unit.

According to one embodiment, the first communication unit of the housing unit is configured for wireless communication with the display device using a standard network protocol. The advantage of the embodiment is that the implementation of the communication units is cheap and the protocols are reliable.

According to one embodiment, the standard network protocol is an NFC-type protocol. The advantage of the embodiment is that the distance between the communicating devices is limited, thereby protecting against eavesdropping attacks.

According to one embodiment, the first communication unit of the housing unit is configured for wireless communication with the display device using a proprietary network protocol. The advantage of the embodiment is that the housing unit is compatible with implantable medical devices that use proprietary network protocols.

According to one embodiment, a communication range of the first communication unit of the housing unit is less than a communication range of the second communication unit of the housing unit. The advantage of the embodiment is that energy is saved by selecting the first communication unit when its range suffices.

According to one embodiment, a communication range of the first communication unit of the display device is less than a communication range of the second communication unit of the display device. The advantage of the embodiment is that energy is saved by selecting the first communication unit when its range suffices.

According to one embodiment, at least one of the housing unit and the display device is configured to allow communication between the housing unit and the display device on the basis of a distance between the housing unit and the display device. The advantage of the embodiment is that the distance is used as a safety and authorization factor.

According to one embodiment, at least one of the housing unit and the display device is configured to allow communication between the housing unit and the display device on the basis of the housing unit being mechanically connected to the display device. The advantage of the embodiment is that the safety against a man-in-the-middle attacks is increased.

According to one embodiment, the housing unit is configured to allow communication between the housing unit and the implantable medical device on the basis of a distance between the housing unit and the implantable medical device. The advantage of the embodiment is that the distance is used as a safety and authorization factor.

According to one embodiment, the housing unit further comprises an encryption unit configured to encrypt communication received from the display device. The advantage of the embodiment is that the encrypted communication is protected against unwanted third party access.

According to one embodiment, the housing unit is further adapted to transmit the encrypted communication to the implantable medical device using the second communication unit. The advantage of the embodiment is that the encrypted communication is protected against unwanted third party access.

According to one embodiment, the second communication unit of the display device is configured to be disabled to enable at least one of: communication between the display device and the housing unit, and communication between the housing unit and the implantable medical device.

The display device in any of the embodiments described herein may be a wearable device or a handset. The advantage of the embodiment is that the device is mobile and can be used where needed.

According to one embodiment, the housing unit comprises a case for the wearable device or handset. The advantage of the embodiment is that the wearable device or handset can be protected from mechanical damage.

Further, a housing unit configured for communication with the implantable medical device when implanted in a patient is provided, the housing unit being configured to mechanically connect to a display device and comprising a first communication unit for communication with the display device and a second communication unit for wireless communication with the implantable medical device.

According to one embodiment, the display device is a wearable device or a handset and the housing unit comprises a case for the wearable device or handset.

According to one embodiment, the first communication unit is a wireless communication unit for wireless communication with the display device.

According to one embodiment, the first communication unit is configured to communicate wirelessly with the display device using a first communication frequency and the second communication unit is configured to communicate wirelessly with the implantable medical device using a second communication frequency, wherein the first and second communication frequencies are different.

According to one embodiment, the housing unit is configured to transmit information pertaining to the display of a user interface to the display device.

According to one embodiment, the housing unit is configured to receive patient input from the display device.

According to one embodiment, the housing unit is configured to display a user interface to the patient.

According to one embodiment, the housing unit is configured to allow communication between the housing unit and the display device on the basis of a distance between the housing unit and the display device.

According to one embodiment, the housing unit is configured to allow communication between the housing unit and the display device on the basis of the housing unit being mechanically connected to the display device.

According to one embodiment, the housing unit is configured to allow communication between the housing unit and the implantable medical device on the basis of a distance between the housing unit and the implantable medical device.

According to one embodiment, the housing unit further comprises an encryption unit configured to encrypt communication received from the display device.

According to one embodiment, the housing unit is further adapted to transmit the encrypted communication to the implantable medical device using the second communication unit.

According to one embodiment, the minimum bounding box of the housing unit and the display device, when the housing is mechanically connected to the display device, is no more than 10% wider, 10% longer or 100% higher than the minimum bounding box of the display device.

According to one embodiment, the housing unit comprises one or more switches configured to be used by the patient when the housing is not mechanically connected to the display device.

According to one embodiment, the switches are at least partly covered by the display device, when the display device is mechanically connected to the housing unit.

According to one embodiment, at least a part of the housing bends in order to mechanically connect to the display device.

According to one embodiment, at least a part of the housing is configured to clasp the display device.

According to one embodiment, the housing is configured to cover at least one side of the display device when it is mechanically connected to the display device.

According to one embodiment, the housing is configured to be mechanically connected to the display device by a device which is mechanically connected to the housing and the display device.

### General Security Module

Further, an implantable controller for the implantable medical device is provided. The implantable controller comprises a wireless transceiver for communicating wirelessly with an external device, a security module, and a central unit configured to be in communication with the wireless transceiver, the security module and the implantable medical device. The wireless transceiver is configured to receive communication from the external device including at least one instruction to the implantable medical device and transmit the received communication to the central unit. The central unit is configured to send secure communication to the security module derived from the communication received from the external device, and the security module is configured to decrypt at least a portion of the secure communication and/or verify the authenticity of the secure communication. The security module is configured to transmit a response communication to the central unit and the central unit is configured to communicate the at least one instruction to the implantable medical device, the at least one instruction being based on the response communication or on a combination of the response communication and the communication received from the external device.

According to one embodiment, the security module comprises a set of rules for accepting communication from the central unit.

According to one embodiment, the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.

According to one embodiment, the set of rules comprises a rule stipulating that communication from the central unit is only accepted when the wireless transceiver has been placed in the off-mode for a specific time period.

According to one embodiment, the central unit is configured to verify a digital signature of the received communication from the external device.

According to one embodiment, the set of rules comprises a rule stipulating that communication from the central unit is only accepted when the digital signature of the received communication has been verified by the central unit.

According to one embodiment, the central unit is configured to verify the size of the received communication from the external device.

According to one embodiment, the set of rules comprises a rule stipulating that communication from the central unit is only accepted when the size of the received communication has been verified by the central unit.

The wireless transceiver of any of the preceding embodiments may be configured to receive a message from the external device being encrypted with at least a first and second layer of encryption and the central unit may be configured to decrypt a first layer of decryption and transmit at least a portion of the message comprising the second layer of encryption to the security model. The security module may be configured to decrypt the second layer of encryption and transmit a response communication to the central unit based on the portion of the message decrypted by the security module.

According to one embodiment, the central unit may be configured to decrypt a portion of the message comprising a digital signature such that the digital signature can be verified by the central unit.

According to one embodiment, the central unit is configured to decrypt a portion of the message comprising message size information such that the message size can be verified by the central unit.

According to one embodiment, the central unit is configured to decrypt a first and second portion of the message, and the first portion comprises a checksum for verifying the authenticity of the second portion.

According to one embodiment, the response communication transmitted from the security module comprises a checksum, and the central unit may be configured to verify the authenticity of at least a portion of the message decrypted by the central unit using the received checksum.

According to one embodiment, the set of rules comprises a rule related to the rate of data transfer between the central unit and the security module.

The security module in any of the embodiments herein may be configured to decrypt a portion of the message comprising a digital signature, encrypted with the second layer of encryption, such that the digital signature can be verified by the security module.

The central unit may be configured such that it is only capable of decrypting a portion of the communication received from the external device when the wireless transceiver is placed in the off-mode.

According to one embodiment, the central unit is only capable of communicating the at least one instruction to the implantable medical device when the wireless transceiver is placed in the off-mode.

According to one embodiment, the implantable controller is configured to receive, using the wireless transceiver, a message from the external device comprising a first non-encrypted portion and a second encrypted portion, decrypt the encrypted portion, and use the decrypted portion to verify the authenticity of the non-encrypted portion.

According to one embodiment, the central unit is configured to transmit the encrypted portion to the security module, receive a response communication from the security module based on information contained in the encrypted portion being decrypted by the security module, and use the response communication to verify the authenticity of the non-encrypted portion.

According to one embodiment, the non-encrypted portion comprises at least a portion of the at least one instruction to the implantable medical device.

The implantable controller may be configured to receive, using the wireless transceiver, a message from the external device comprising information related to at least one of a physiological parameter of the patient and a physical parameter of the implanted medical device and use the received information to verify the authenticity of the message.

The physiological parameter of the patient may comprise at least one of: a temperature, a heart rate and a saturation value.

The physical or functional parameter of the implanted medical device may comprise at least one of: a current setting or value of the implanted medical device, a prior instruction sent to the implanted medical device and an ID of the implanted medical device.

According to one embodiment, the portion of the message comprising the information is encrypted, and the central unit is configured to transmit the encrypted portion to the security module and receive a response communication from the security module based on the information having been decrypted by the security module.

According to one embodiment, the security module comprises a hardware security module comprising at least one hardware-based key. The hardware-based key may correspond to a hardware-based key in the external device, which may be a hardware-based key on a key-card connectable to the external device.

According to one embodiment, the security module comprises a software security module comprising at least one software-based key. The software-based key may correspond to a software-based key in the external device. The software-based key may correspond to a software-based key on a key-card connectable to the external device. The security module may in any of the embodiments comprise a combination of a software-based key and a hardware-based key.

In any of the preceding embodiments, the implantable controller may comprise at least one crypto-processor.

The wireless transceiver may in any of the embodiments be configured to receive communication from a handheld external device.

According to one embodiment, the at least one instruction to the implantable medical device may comprise an instruction for changing an operational state of the implantable medical device.

The wireless transceiver may be configured to communicate wirelessly with the external device using electromagnetic waves at a frequency below 100 kHz or at a frequency below 40 kHz.

According to one embodiment, the wireless transceiver is configured to communicate wirelessly with the external device using a first communication protocol, and the central unit is configured to communicate with the security module using a second different communication protocol.

In any of the embodiments, the wireless transceiver may be configured to communicate wirelessly with the external device using a standard network protocol. The standard network protocol may be selected from a list comprising RFID-type protocols, WLAN-type protocols, Bluetooth type protocols, BLE-type protocols, NFC-type protocols, 3G/4G/5G-type protocols, and GSM-type protocols.

The wireless transceiver may in some embodiments be configured to communicate wirelessly with the external device using a proprietary network protocol.

According to one embodiment, the wireless transceiver comprises a UWB transceiver.

According to one embodiment, the security module and/or the central unit and/or the wireless transceiver are comprised in the controller.

The external unit in any of the embodiments herein may be a wearable device or a handset. The advantage of the embodiment is that the device is mobile and can be used where needed.

Further, the implantable medical device may comprise a receiving unit. The implantable medical device comprises at least one coil configured for receiving transcutaneously transferred energy, a measurement unit configured to measure a parameter related to the energy received by the coil, a variable impedance electrically connected to the coil, a switch placed between the variable impedance and the coil for switching off the electrical connection between the variable impedance and the coil. The implantable medical device further comprises a controller configured to control at least one of the variable impedance for varying the impedance and thereby tune the coil based on the measured parameter, and the switch for switching off the electrical connection between the variable impedance and the coil in response to when the measured parameter exceeds a threshold value.

According to one embodiment, the controller is configured to vary the variable impedance in response to when the measured parameter exceeds a threshold value.

According to one embodiment, the measurement unit is configured to measure a parameter related to the energy received by the coil over a time period.

According to one embodiment, the measurement unit is configured to measure a parameter related to a change in energy received by the coil.

According to one embodiment, the first switch is placed at a first end portion of the coil, and the implantable medical device further comprises a second switch placed at a second end portion of the coil such that the coil can be completely disconnected from other portions of the implantable medical device.

According to one embodiment, the receiving unit is configured to receive transcutaneously transferred energy in pulses according to a pulse pattern, and the measurement unit is configured to measure a parameter related to the pulse pattern.

According to one embodiment, the controller is configured to control the variable impedance in response to when the pulse pattern deviates from a predefined pulse pattern.

According to one embodiment, the controller is configured to control the switch for switching off the electrical connection between the variable impedance and the coil in response to the pulse pattern deviating from a predefined pulse pattern.

According to one embodiment, the measurement unit is configured to measure a temperature in the implantable medical device or in the body of the patient, and the controller is configured to control the first and second switch in response to the measured temperature.

According to one embodiment, the variable impedance comprises a resistor and a capacitor, a resistor and an inductor and/or an inductor and a capacitor.

The variable impedance may comprise a digitally tuned capacitor. The variable impedance may comprise a digital potentiometer. The variable impedance may comprise a variable inductor.

According to one embodiment, the variation of the impedance is configured to lower the active power that is received by the receiving unit.

According to one embodiment, the variable impedance is placed in series with the coil.

According to one embodiment, the variable impedance is placed parallel to the coil.

According to one embodiment, the implantable medical device further comprises an energy storage unit connected to the receiving unit. The energy storage unit is configured to store energy received by the receiving unit.

As mentioned before, the system as described above is particularly useful for use in a valve such as an artificial sphincter. Therefore, according to another aspect of the present disclosure, an artificial sphincter may be configured, when implanted, to act on a wall of an intestine of a patient so as to restrict flow of intestinal contents out of the intestine and may comprise a system as described herein.

Likewise, the system as described above is particularly useful for use in an emptying device. Therefore, according to another aspect of the present disclosure, an emptying device may be configured, when implanted, to act on a wall of an intestine of a patient so as to advance intestinal contents contained in the intestine out of the intestine and may comprise a system as described herein.

### Implantation

Accordingly, a method of implanting a system for treating a patient having a disorder related to the patient's intestine comprises the steps of:
making an incision in the body of the patient for accessing the intestine,
inserting one or more electrical stimulation devices, wherein each of the one or more electrical stimulation devices comprises one or more electrodes for electrically stimulating muscle or neural tissue of the intestine and a wireless energy receiver configured to receive energy for stimulating the muscle or neural tissue wirelessly,
placing the electrodes of the one or more electrical stimulation devices in connection with the intestine,
inserting one or a plurality of wireless energy transmitters,
placing the one or plurality of wireless energy transmitters in proximity to the one or more electrical stimulation devices so as to allow transfer of energy from the one or plurality of energy transmitters to all of the one or more electrical stimulation devices.

The method of implanting the system may comprise further steps as described above and in more detail hereinafter.

A method of using the system, the artificial sphincter or the emptying device accordingly comprises the step of wirelessly transmitting energy to and receiving the energy by the energy receiver. The method of using the system may comprise further steps as described above and in more detail hereinafter.

### Surface Coating

A further aspect of the present disclosure relates to the mitigation of fibrin creation caused by contact between a medical implant, such as the above-discussed implantable system, and the tissue or flowing blood of a patient. As is well known, the body tends to react to a medical implant, partly because the implant is a foreign object, and partly because the implant interacts mechanically with tissue of the body and/or blood flowing within the body. Implantation of medical devices and/or biomaterial in the tissue of a patient may trigger the body's foreign body reaction leading to the formation of foreign body giant cells and the development of a fibrous capsule enveloping the implant. The formation of a dense fibrous capsule that isolates the implant from the host is the common underlying cause of implant failure. Implantation of medical devices and/or biomaterial in a blood flow may also cause the formation of fibrous capsules due to the attraction of certain cells within the blood stream. Implants may, due to the fibrin formation, cause blood clotting leading to complications for the patient. Implants in contact with flowing blood and/or placed in the body may also lead to bacterial infection. One common way of counteracting the creation of blood clots is by using blood thinners of different sorts. One commonly used blood thinner is called heparin. However, heparin has certain side effects that are undesirable.

In general, fibrin is an insoluble protein that is partly produced in response to bleeding and is the major component of blood clots. Fibrin is formed by fibrinogen, a soluble protein that is produced by the liver and found in blood plasma. When tissue damage results in bleeding, fibrinogen is converted at the wound into fibrin by the action of thrombin, a clotting enzyme. The fibrin then forms, together with platelets, a hemostatic plug or clot over a wound site. The process of forming fibrin from fibrinogen starts with the attraction of platelets. Platelets have thrombin receptors on their surfaces that bind serum thrombin molecules. These molecules can in turn convert soluble fibrinogen into fibrin. The fibrin then forms long strands of tough and insoluble protein bound to the platelets. The strands of fibrin are then cross-linked so that it hardens and contracts. This is enabled by Factor XIII which is a zymogen found in the blood of humans. Fibrin may also be created due to the foreign body reaction. When a foreign body is detected in the body, the immune system will become attracted to the foreign material and attempt to degrade it. If this degradation fails, an envelope of fibroblasts may be created to form a physical barrier to isolate the body from the foreign body. This may further evolve into a fibrin sheath. In case the foreign body is an implant, this may hinder the function of the implant.

Thus, implants can, when implanted in the body, be in contact with flowing blood. This may cause platelet adhesion on the surface of the implants. The platelets may then cause the fibrinogen in the blood to convert into fibrin creating a sheath on and/or around the implant. This may prevent the implant from working properly and may also create blood clots that are perilous for the patient. However, implants not in contact with flowing blood can still malfunction due to fibrin creation. Here the foreign body reaction may be the underlying factor for the malfunction. Further, the implantation of a foreign body into the human body may cause an inflammatory response. The response generally persists until the foreign body has been encapsulated in a relatively dense layer of fibrotic connective tissue which protects the human body from the foreign body. The process may start with the implant immediately and spontaneously acquiring a layer of host proteins. The blood protein-modified surface enables cells to attach to the surface, enabling monocytes and macrophages to interact on the surface of the implant. The macrophages secrete proteins that modulate fibrosis and in turn develop the fibrosis capsule around the foreign body, i.e., the implant. In practice, a fibrosis capsule may be formed of a dense layer of excess fibrous connective tissue. The inelastic properties of the fibrotic capsule may lead to hardening, tightness, deformity, and distortion of the implant, which in severe cases may result in revision surgery.

Implants may also cause infections of different sorts. Bacterial colonization that leads to implant-associated infections are a known issue for many types of implants. For example, the commensal skin bacteria. Staphylococci, and the Staphylococcus aureus tend to colonize foreign bodies such as implants and may cause infections. A problem with the Staphylococci is that it may also produce a biofilm around the implant encapsulating the bacterial niche from the outside environment. This makes it harder for the host defense systems to take care of the bacteria. There are other examples of bacteria and processes that creates bacteria causing infection due to implants.

Thus, according to this further aspect of the present disclosure, in order to mitigate fibrin creation caused by contact between components of the above-discussed implantable system, and the tissue or flowing blood of a patient, the implantable components of the system may comprise a specific coating arranged on the respective outer surface of the component. The coating may comprise at least one layer of a biomaterial. The biomaterial is preferably fibrin-based. The coating may comprise at least one drug or substance with antithrombotic and/or antibacterial and/or antiplatelet characteristics. The drug or substance may be encapsulated in a porous material.

There may be provided a second coating arranged on the first coating. The second coating may be a different biomaterial than said first coating. In particular, the first coating may comprise a layer of perfluorocarbon chemically attached to the surface and the second coating may comprise a liquid perfluorocarbon layer.

Further preferably, the surface may comprise a metal, such as at least one of titanium, cobalt, nickel, copper, zinc, zirconium, molybdenum, tin or lead.

Finally, the surface may comprise a micro pattern, wherein the micro pattern may be etched into the surface prior to insertion into the body. The layer of a biomaterial may be coated on the micro pattern.

A further aspect of the present disclosure relates to an implantable energized medical device, which may advantageously be combined with the disclosed system for treating a patient having a disorder related to a patient's intestine and which is configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the connecting portion and second portion are configured to form a connecting interface between the connecting portion and the second portion, and the second portion extends along a first direction being parallel to the second plane, wherein the second portion has a lengthwise cross-sectional area along the first direction, wherein a second lengthwise cross-sectional area is smaller than a first lengthwise cross-sectional area and wherein the first lengthwise cross-sectional area is located closer to said connecting interface with regard to the first direction.

In some embodiments, the second portion has a first end and a second end opposing the first end along the first direction, wherein the second portion has a length between the first and second end, and wherein the second portion has an intermediate region and a distal region, wherein the intermediate region is defined by the connecting interface between the connecting portion and the second portion, and the distal region extends from the connecting interface between the connecting portion and the second portion to the second end.

In some embodiments, the lengthwise cross-sectional area of the second portion decreases continuously from an end of the intermediate region towards the second end.

In some embodiments, the lengthwise cross-sectional area of the second portion decreases linearly from an end of the intermediate region towards the second end.

In some embodiments, the lengthwise cross-sectional area of the second portion decreases stepwise from an end of the intermediate region towards the second end.

In some embodiments, the distal region of the second portion is conically shaped.

In some embodiments, the second portion has rotational symmetry along the first direction.

In some embodiments, the second surface of the second portion is substantially perpendicular to a central extension of the connecting portion.

In some embodiments, the second surface of the second portion is substantially parallel to the second plane.

In some embodiments, the second surface of the second portion is substantially flat and configured to form a contact area to the second tissue surface, and wherein the second portion further comprises a lower surface facing away from the first portion configured to taper towards the second end.

In some embodiments, the second portion has a proximal region, wherein the proximal region extends from the first end to the connecting interface between the connecting portion and the second portion.

In some embodiments, the lengthwise cross-sectional area of the second portion decreases continuously from an end of the intermediate region towards the first end.

In some embodiments, the lengthwise cross-sectional area of the second portion decreases linearly from an end of the intermediate region towards the first end.

In some embodiments, the lengthwise cross-sectional area of the second portion decreases stepwise from an end of the intermediate region towards the first end.

In some embodiments, the proximal region of the second portion is conically shaped.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the second portion has said length in a direction being different to a central extension of the connecting portion.

In some embodiments, the connecting interface between the connecting portion and the second portion is eccentric with respect to the second portion.

In some embodiments, the connecting interface between the connecting portion and the second portion is eccentric, with respect to the second portion, in the first direction, but not in a second direction being perpendicular to the first direction.

In some embodiments, the connecting interface between the connecting portion and the second portion is eccentric, with respect to the second portion, in the first direction and in a second direction being perpendicular to the first direction.

In some embodiments, the second direction is parallel to the second plane.

In some embodiments, the proximal region and the distal region comprises the second surface configured to engage the second surface of the second side of the tissue portion.

In some embodiments, the second portion is tapered from the first end to the second end.

In some embodiments, the second portion is tapered from the intermediate region of the second portion to each of the first end and second end.

In some embodiments, the first portion has a maximum dimension being in the range of 10 to 40 mm, such as in the range of 10 to 30 mm, such as in the range of 15 to 25 mm.

In some embodiments, the first portion has a diameter being in the range of 10 to 40 mm, such as in the range of 10 to 30 mm, such as in the range of 15 to 25 mm.

In some embodiments, the connecting portion has a maximum dimension in the third plane in the range of 2 to 20 mm, such as in the range of 2 to 15 mm, such as in the range of 5 to 10 mm.

In some embodiments, the second portion has a maximum dimension being in the range of 30 to 90 mm, such as in the range of 30 to 70 mm, such as in the range of 35 to 60 mm.

In some embodiments, the first portion has one or more of a spherical shape, an ellipsoidal shape, a polyhedral shape, an elongated shape, and a flat disk shape.

In some embodiments, the connecting portion has one of an oval cross-section, an elongated cross-section, and a circular cross-section, in a plane parallel to the third plane.

In some embodiments, the distal region is configured to be directed downwards in a standing patient.

In some embodiments, the first portion has a first height, and the second portion has a second height, both heights being in a direction perpendicular to the first and second planes, wherein the first height is smaller than the second height.

In some embodiments, the first height is less than ⅔ of the second height, such as less than ½ of the second height, such as less than ⅓ of the second height.

In some embodiments, the second end of the second portion comprises connections for connecting to an implant being located in a caudal direction from a location of the implantable energized medical device in the patient.

In some embodiments, the first end of the second portion comprises connections for connecting to an implant being located in a cranial direction from a location of the implantable energized medical device in the patient.

In some embodiments, the connecting portion further comprises a fourth cross-sectional area in a fourth plane, wherein the fourth plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the first surface is configured to engage the first tissue surface of the first side of the tissue portion.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the velocity and increase the force of the movement generated by the electrical motor.

In some embodiments, the transmission is configured to transfer a week force with a high velocity into a stronger force with lower velocity.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the implantable energized medical device further comprises a capacitor connected to at least one of the first and second energy storage unit and connected to the electrical motor, wherein the capacitor is configured to: be charged by at least one of the first and second energy storage units, and provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical device further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

In some embodiments, the fourth plane is parallel to a major extension plane of the tissue.

A further aspect of the present disclosure relates to an implantable energized medical device, which may advantageously be combined with the disclosed system for treating a patient having a disorder related to a patient's intestine and which is configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive electromagnetic waves at a frequency above a frequency level, and/or to transmit electromagnetic waves at a frequency below the frequency level, wherein the second portion is configured to receive and/or transmit electromagnetic waves at a frequency below the frequency level, and wherein the frequency level is 100 kHz.

In some embodiments, wherein the first portion is configured to transmit electromagnetic waves at the frequency below the frequency level to the second portion.

In some embodiments, the first portion is configured to transmit electromagnetic waves at the frequency above the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter above the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device above the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

A further aspect of the present disclosure relates to an implantable energized medical device, which may advantageously be combined with the disclosed system for treating a patient having a disorder related to a patient's intestine and which is configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive and/or transmit electromagnetic waves at a frequency below the frequency level, and wherein the frequency level is 100 KHz.

In some embodiments, the second portion is configured to receive and/or transmit electromagnetic waves at a frequency below the frequency level.

In some embodiments, the first portion is configured to transmit electromagnetic waves at the frequency below the frequency level to the second portion.

In some embodiments, the first portion is configured to transmit electromagnetic waves at the frequency below the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter below the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device below the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the first portion comprises an outer casing made from titanium.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

A further aspect of the present disclosure relates to an implantable energized medical device, which may advantageously be combined with the disclosed system for treating a patient having a disorder related to a patient's intestine and which is configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is made from a polymer material, the second portion comprises a casing made from titanium, wherein the casing forms a complete enclosure.

In some embodiments, the casing of the second portion forms a complete enclosure such that the entirety of the outer surface of the second portion is covered by the casing, when the second portion is connected to the connecting portion.

In some embodiments, the first portion comprises a casing made from the polymer material.

In some embodiments, the casing of the first portion forms a complete enclosure such that the entirety of the outer surface of the first portion is covered by the casing.

In some embodiments, the connecting portion comprises a connection arranged to connect to the first and second portion respectively and carry electrical signals and/or energy.

In some embodiments, the connection is arranged in a core of the connecting portion such that it is encapsulated by outer material of the connecting portion.

In some embodiments, the connecting portion comprises a ceramic material.

In some embodiments, the connection is encapsulated within the ceramic material.

In some embodiments, the first portion comprises a first connection configured to connect to the connection of the connecting portion.

In some embodiments, the second portion comprises a second connection configured to connect to the connection of the connection portion.

In some embodiments, the casing of the second portion is hermetically sealed.

In some embodiments, the second connection is arranged such that the hermetical seal of the second portion is kept intact.

In some embodiments, the casing of the first portion is hermetically sealed.

A further aspect of the present disclosure relates to an implantable energized medical device, which may advantageously be combined with the disclosed system for treating a patient having a disorder related to a patient's intestine and which is configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, and wherein the connecting portion is configured to extend between the first portion and the second portion along a central extension axis, and wherein the second portion is configured to extend in a length direction being divergent with the central extension axis, and wherein the connecting portion has a substantially constant cross-sectional area along the central extension axis, or wherein the connecting portion has a decreasing cross-sectional area in a direction from the first portion towards the second portion along the central extension axis, and/or wherein the second portion has a substantially constant cross-sectional area along the length direction, or wherein the second portion has a decreasing cross-sectional area in the length direction.

In some embodiments, the third cross-sectional area is smaller than the first cross-sectional area.

In some embodiments, the connecting portion is tapered in the direction from the first portion towards the second portion along the central extension axis.

In some embodiments, the connecting portion has a circular or oval cross-section along the central extension axis with a decreasing diameter in the direction from the first portion towards the second portion.

In some embodiments, the second portion is tapered in the length direction.

In some embodiments, the connecting portion has a circular or oval cross-section in the length direction with a decreasing diameter in the length direction.

In some embodiments, the length direction extends from an interface between the connecting portion and the second portion towards an end of the second portion.

In some embodiments, the length direction extends in a direction substantially perpendicular to the central extension axis.

According to an embodiment of the present inventive concept, an implantable device for exerting a force on a body portion of a patient is provided, the implantable device comprising: an implantable energized medical device and an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

### BRIEF DESCRIPTION OF THE DRAWINGS

The invention is now described, by way of example, with reference to the accompanying drawing, in which:

FIG. 1A shows a first embodiment of a system for electrically stimulating tissue of a patient's intestine in a top view.

FIG. 1B shows the system of FIG. 1A in a—partially cross-sectional-side view.

FIG. 1C shows the system of FIG. 1A in a different side view.

FIG. 2A shows a second embodiment of a system for electrically stimulating tissue of a patient's intestine in a top view.

FIG. 2B shows the system of FIG. 2A in a—partially cross-sectional-side view.

FIG. 3A shows a third embodiment of a system for electrically stimulating tissue of a patient's intestine in a top view.

FIG. 3B shows the system of FIG. 3A in a—partially cross-sectional-side view.

FIG. 4A shows a fourth embodiment of a system for electrically stimulating tissue of a patient's intestine in a top view.

FIG. 4B shows the system of FIG. 4A in a—partially cross-sectional-side view.

FIG. 5A shows a fifth embodiment of a system for electrically stimulating tissue of a patient's intestine in a top view.

FIG. 5B shows the system of FIG. 5A in a—partially cross-sectional-side view.

FIG. 5C shows a variant of the system of FIG. 5A in a—partially cross-sectional-side view.

FIG. 6 shows a sixth embodiment of a system for electrically stimulating tissue of a patient's intestine in a top view.

FIG. 7 shows a seventh embodiment of a system for electrically stimulating tissue of a patient's intestine in a top view.

FIG. 8 shows an eighth embodiment of a system for electrically stimulating tissue of a patient's intestine in a top view.

FIG. 9 shows a ninth embodiment of a system for electrically stimulating tissue of a patient's intestine in a top view.

FIG. 10A shows a hydraulic pump system in a top view which may be provided to support any one of the embodiments of a system for electrically stimulating tissue of a patient's intestine as described herein.

FIG. 10B shows the system of FIG. 10A in a—partially cross-sectional-side view.

FIG. 11 shows an eleventh embodiment of a system for electrically stimulating tissue of a patient's intestine in a top view.

FIGS. 12A-D show various examples of electrode arrangements for electrically stimulating muscle tissue of the patient.

FIGS. 13 and 14 illustrate a pulsed signal for electrically stimulating muscle tissue.

FIGS. 15 to 17 are schematic illustrations of systems for treating reflux disease.

FIG. 18 is a flow chart of a method of implantation of the system:

FIGS. 19A, 19B, 19B′ and 19C generally illustrate a system for communication with an implanted medical device:

FIG. 20 shows an embodiment of a system for charging, programming and communicating with a controller of an implanted medical device:

FIG. 21 shows an elevated perspective view from the left of a housing unit;

FIG. 22 shows a plan view from the left of a housing unit:

FIG. 23 shows an elevated perspective view from the left of a housing unit:

FIG. 24 shows a plan view from the left of a housing unit:

FIG. 25 shows a system overview of an external device comprising a housing unit and a display device in wireless communication with an implanted medical device:

FIG. 26 shows an implant with an implant surface and a coating arranged on the surface:

FIG. 27 shows an implant with an implant surface and multiple coatings arranged on the surface:

FIGS. 28A and 28B show different micro patterns on the surface of an implant:

FIGS. 29 and 30 show an embodiment of an implantable energized medical device;

FIGS. 31A to 31D show a first portion and a connecting portion of the medical device of FIGS. 29 and 30;

FIGS. 32A to 34B show variants of an element of the connecting portion of FIGS. 31A to 31C:

FIG. 35 shows a kit for assembling the medical device of FIGS. 29 and 30;

FIG. 36 shows a further embodiment of an implantable energized medical device;

FIG. 37 shows a general example of an implantable energized medical device:

FIG. 38 shows a first variant of the general example of the medical device of FIG. 37;

FIG. 39 shows a second variant of the general example of the medical device of FIG. 37;

FIGS. 40A and 40B show cross sections of the medical device of FIG. 37:

FIGS. 41A to 41Q show different relative arrangements of first and second parts of the medical device of FIG. 37:

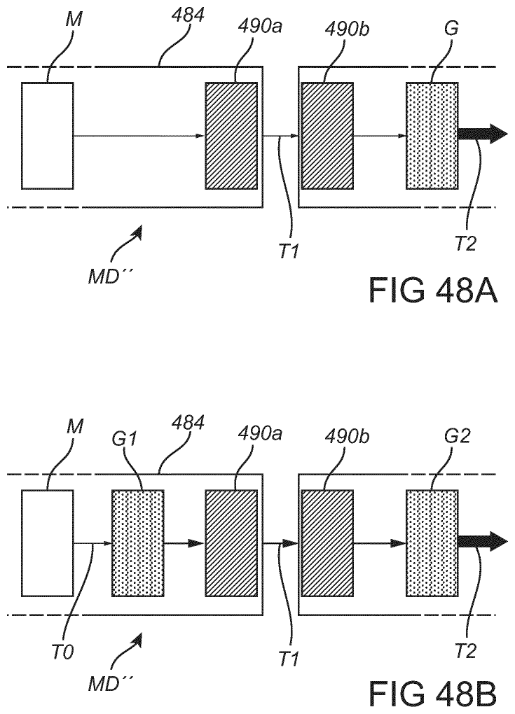

FIGS. 37 and 43 show a third variant of the general example of the medical device of FIG. 37:

FIGS. 44 and 45 show the medical device of FIG. 37 with first and second parts thereof being differently rotationally displaced relative to each other:

FIGS. 46A to 46C illustrate a procedure of inserting the medical device of FIGS. 37 and 43;

FIG. 47 shows an even further embodiment of an implantable energized medical device:

FIGS. 48A and 48B illustrate a gear arrangement and magnetic coupling for coupling the implantable energized medical device to an implant:

FIG. 49A shows a perspective elevated view from the right of an embodiment of an implantable energized medical device for powering an implantable medical device:

FIGS. 49B and 49C show lengthwise cross-sectional areas of the implantable medical device along the line A-A in FIG. 49A:

FIGS. 50 to 52 show cross-sectional plain side views of embodiments of an implantable energized medical device for powering an implantable medical device:

FIG. 53A shows a perspective elevated view from the right of an embodiment of an implantable energized medical device for powering an implantable medical device:

FIGS. 53B and 53C show lengthwise cross-sectional areas of the implantable medical device along the line A-A in FIG. 53A.

### DETAILED DESCRIPTION

In the following, a detailed description of embodiments of the invention will be given with reference to the accompanying drawings. It will be appreciated that the drawings are for illustration only and are not in any way restricting the scope of the invention. Thus, any references to directions, such as “up” or “down”, are only referring to the directions shown in the Figures. It should be noted that the features having the same reference numerals have the same function, a feature in one embodiment may thus be exchanged for a feature from another embodiment having the same reference numeral unless clearly contradictory. The descriptions of the features having the same reference numerals are thus to be seen as complementing each other in describing the fundamental idea of the feature and thereby showing the feature's versatility.

Restriction of the intestine is to be understood as any operation decreasing a cross-sectional area of the intestine. The restriction may decrease the flow of matter in the intestine or may completely close the intestine such that no matter can pass. Constriction is to be understood as a special way of restricting the intestine, namely a restriction by constriction, e.g. by means of a mechanical or hydraulic constriction device acting on the intestine from its outside and thereby constricting it.

A controller is to be understood as any unit capable of controlling at least a part of the system. A controller may include a motor and/or pump or any another operational device for operating at least part of the system. It may be separate from the electrical stimulation device and/or mechanical or hydraulic constriction device and may be adapted to control only the operation thereof. Preferably, a controller includes a CPU which enables the controller to process data. A control signal is to be understood as any signal capable of carrying information and/or electric power such that the electrical stimulation device and/or mechanical or hydraulic constriction device or any other part of the system can be controlled directly or indirectly.

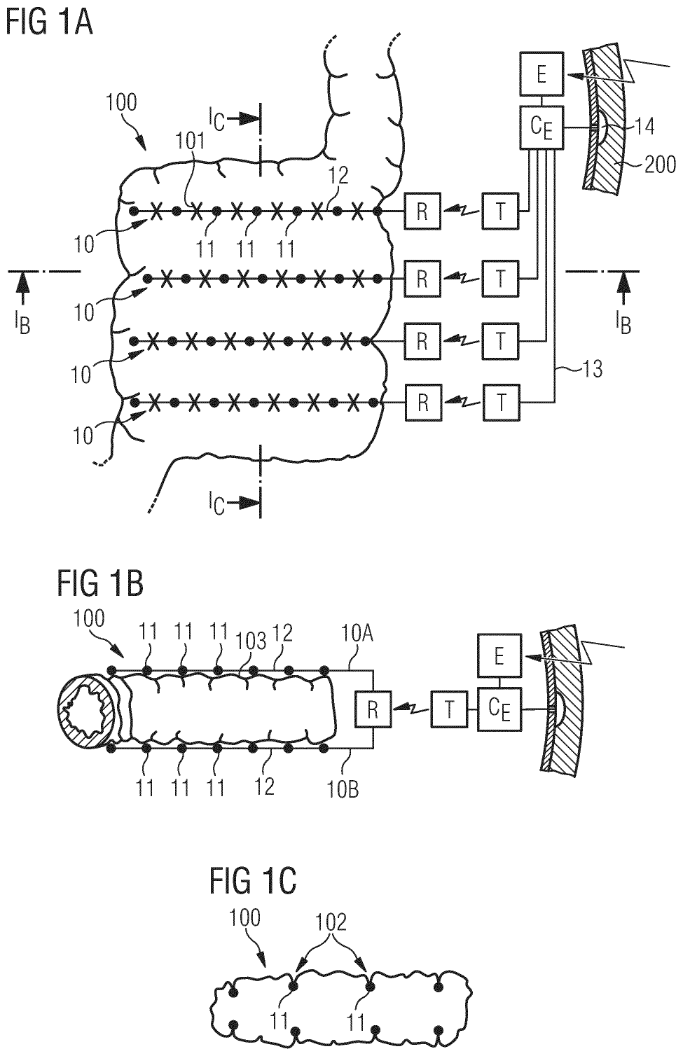

FIG. 1A shows a top view of a first embodiment of a system for treating a patient having a disorder related to a patient's intestine 100. In this embodiment as well as in the following embodiments, the system is adapted to be implanted in relation to a reservoir section of an intestine 100 which is formed from surgically modified intestine that has been cut along a mutual contact line of laterally adjacent sections of a bent portion of intestine and connected so that the upper and lower halves of the cut intestine form an intestinal wall of the reservoir section. The connection lines are sewn together by sutures 101.

The system for treating the patient's intestine involves electrical stimulation thereof by a plurality of electrical stimulation devices 10. Each of the electrical stimulation devices 10 may comprise one or more electrodes 11. In the embodiment shown, an electrical stimulation device 10 comprises seven electrodes 11. The electrodes 11 in each of the electrical stimulation devices 10 may be interconnected by an electrical wire 12, which means that those electrodes 11 are energized simultaneously when a voltage is applied to the wire 12. In the embodiment shown, the electrical wire 12 with the electrodes 11 connected thereto is arranged along the mutual contact line where the upper and lower halves of the cut intestine are sewn together by sutures 101. Alternatively, the electrodes 11 may be arranged at different locations of the intestine 100.

Each one of the electrical stimulation devices 10 comprises a wireless energy receiver R configured to receive energy for wirelessly stimulating the muscle or neural tissue of the intestine 100. Thus, the electrical stimulation devices 100 are not physically interconnected but are independent from each other. As can be seen from the side view shown in FIG. 1B, the electrical stimulation device 10 includes two branches 10A, 10B which share a common wireless energy receiver R. As can be seen in a different view shown in FIG. 1C, the electrodes 11 may be arranged in surgically created folds 102, either with or without the electrical wire 12. Alternatively, the electrodes 11 and/or the electrical wire 12 may be attached to an outside wall 103 of the intestine 100, as shown in FIG. 1B, or may be implanted in the wall 103 (not shown).

In the embodiment shown in FIGS. 1A to IC, a wireless energy transmitter T is provided for each one of the respective wireless energy receivers R. Thus, energy is transmitted wirelessly to the electrical stimulation devices 10, which makes the electrical stimulation devices 10 relatively independent not only from each other but also from the remaining part of the overall system. This way, the electrical stimulation devices 10 remain rather flexible over time since the danger of decreased flexibility due to fibrosis growing over and encapsulating the system is minimized. This is important for a proper functioning of the intestine which must be able to undergo filling and emptying movements, in particular peristaltic movements. This applies not only to an intestinal reservoir as modified and shown in e.g. FIG. 1, but also to regular intestines to which the systems as disclosed herein are likewise applicable. However, what needs to be taken care of is that the wireless energy transmitters T are arranged, more particularly implanted, sufficiently close to the wireless energy receivers R such that energy can safely be transmitted from the energy transmitters T to the respectively associated energy receivers R.

Energy transfer between the wireless energy transmitters T and the wireless energy receivers R is preferably carried out via cooperating antennas, such as a primary coil on each of the transmitters T and a secondary coil on each of the receivers R, wherein the primary coils are configured to induce a voltage in the associated secondary coil, for which reason the wireless energy transmitters and receivers should be arranged close to each other, when implanted.

The primary and secondary coils of the wireless transmitters T and receivers R allow for using RFID technology to transfer the energy from the energy transmitter to the energy receiver. This technology is well established. In particular, the wireless energy receivers R may be configured to receive the energy via RFID pulses.

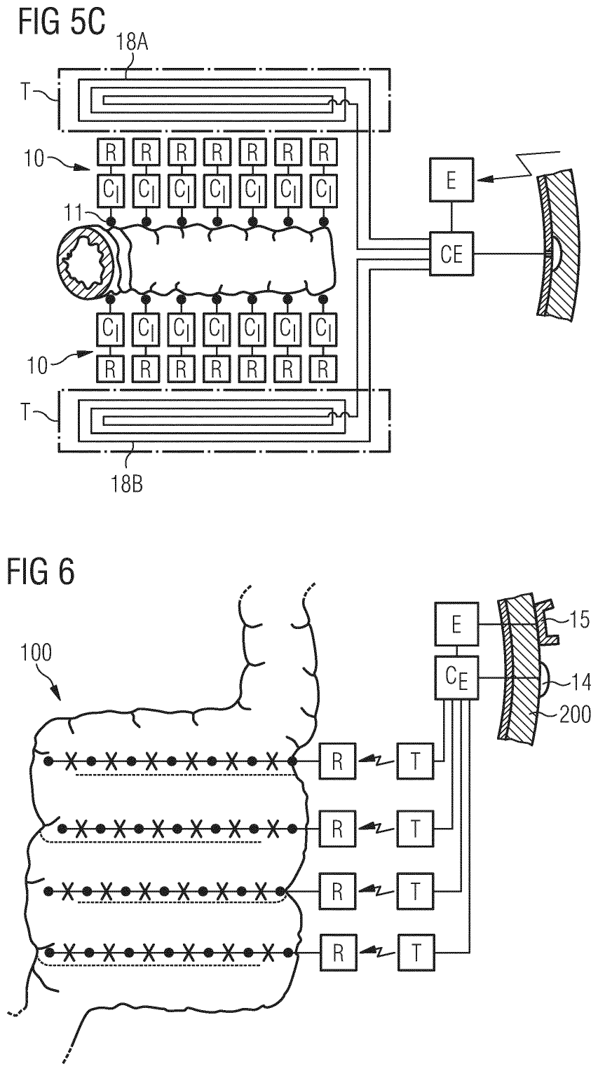

In turn, the wireless energy transmitters T do not necessarily need to maintain flexibility over time and, therefore, they are each connected to a controller via electric wiring 13. The controller is referenced with C₁₁ representing an “external” controller as compared to an internal controller which may make part of the electrical stimulation devices 10, as will be described herein after. More specifically, the external controller C₁₁ is an implanted external controller. Here, implantation is under the skin such that it can be actuated manually by means of a switch 14, which may have the form of a press button. In particular, the switch 14 may be implanted under the skin, as shown in FIG. 1A, or may be provided on the patient's skin outside the patient's body (as shown in FIG. 6).

Furthermore, an energy storage unit E, which is rechargeable, is connected to the external controller C₁₁ so as to provide energy to the wireless energy transmitters T when controlled accordingly by the external controller C₁₁. The energy storage unit is rechargeable wirelessly through the patient's skin 200, as indicated in FIG. 1A by an arrow, for which reason the energy storage unit E is preferably implanted very close to the patient's skin 200. Alternatively, as shown in FIG. 6, the energy storage unit E may be connected by wire to a port 15 mounted on the patient's skin 200. Whenever needed, the energy storage unit E may be recharged by docking an electric charger to the port 15.

Accordingly, when the system is implanted and used by a patient or by a care person, one may actuate the switch 14 implanted underneath the skin 200 by pressing thereon, which initiates the controller C₁₁ so as to run a program installed in a CPU of the controller C₁₁. According to such program, the controller C₁₁ will release energy from the energy storage unit E sequentially to the electrical stimulation devices 10. Consequently, different parts of the intestine 100 are electrically stimulated at different times so that they contract and, thereby, restrict the volume inside the intestine 100. This way, intestinal contents contained inside the intestine 100 may be urged further and further through the intestine 100 towards an end of the intestine 100. At the end of the program, energy transfer between the wireless energy transmitters T and receivers R is terminated so that the neural and muscle tissue of the intestine 100 may relax. Of course, the running of the program in the external controller C₁₁ can be interrupted at any time by actuating the switch 14 once again, if desired.

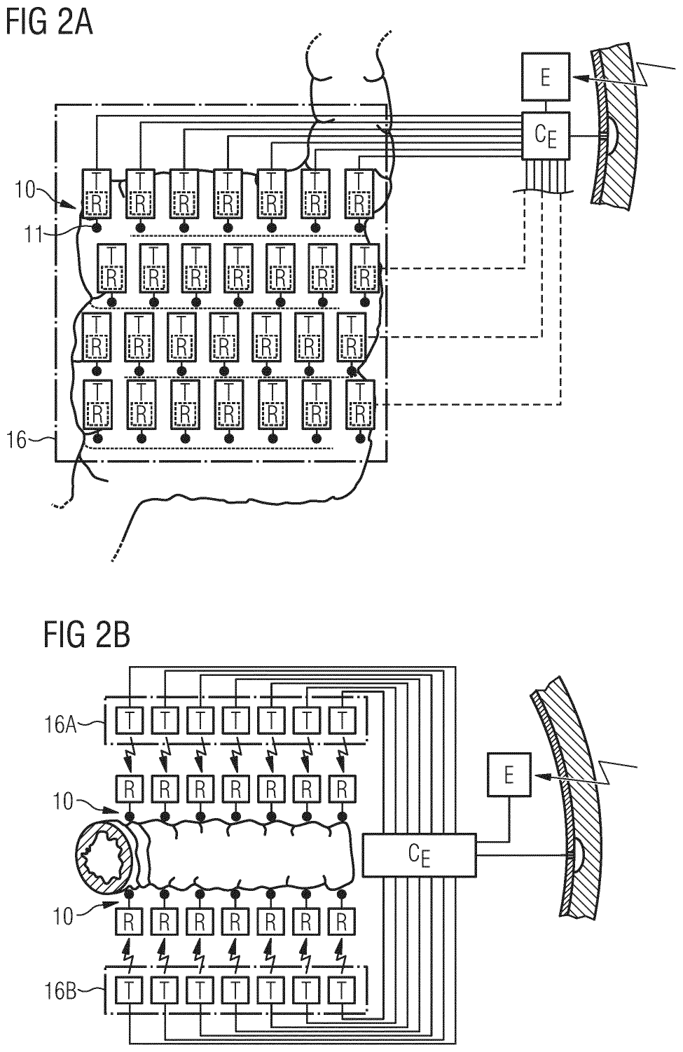

FIG. 2A shows a second embodiment of a system for electrically stimulating tissue of a patient's intestine in a top view. This embodiment differs from the first embodiment in that each electrical stimulation device 10 includes a single electrode 11 only. Accordingly, since each electrical stimulation device 10 has its own wireless energy receiver R, there is provided a wireless energy transmitter T for each one of the electrical stimulation devices 10. The wireless energy transmitters T may be arranged on a common web 16, which may have a net-like structure with one wireless energy transmitter T being preferably mounted to an associated connecting point of the net-like structure.

FIG. 2B shows the system of FIG. 2A in a side view and with two of the aforementioned webs 16, referenced in FIG. 2B as webs 16A and 16B, similar to the branches 10A and 10B in FIG. 1B. As can be seen from both FIGS. 2A and 2B, the wireless energy transmitters T overlie the respectively associated wireless energy receivers R with a minimal distance so as to provide the best energy transfer between the wireless energy transmitters T and receivers R.

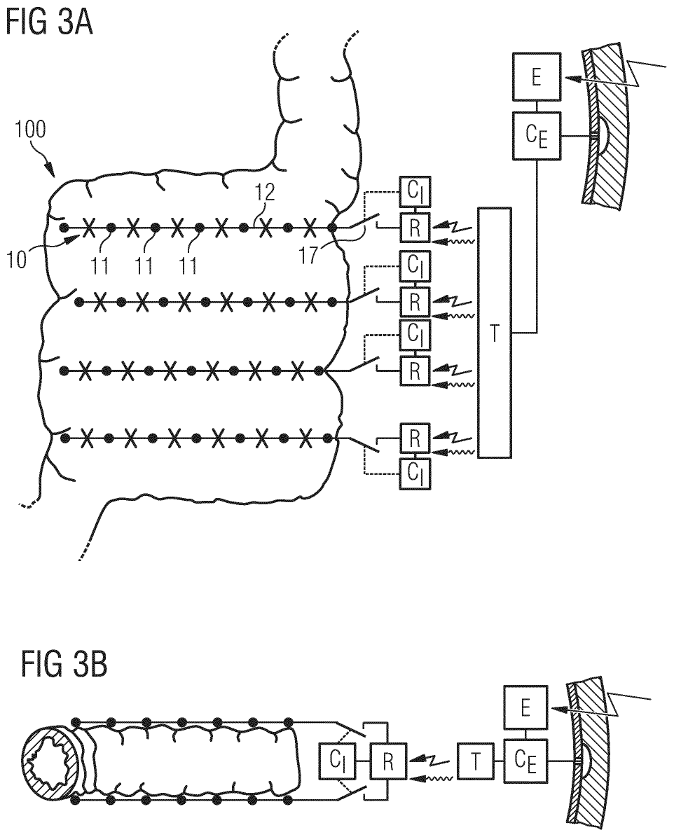

FIG. 3A shows a third embodiment of a system for electrically stimulating tissue of a patient's intestine in a top view. FIG. 3B shows a side view of the third embodiment. This embodiment differs from the first embodiment shown in FIGS. 1A to IC in that a single wireless energy transmitter T is provided to transmit energy to all of the electrical stimulation devices 10. Since it is not desired to energize all electrical stimulation devices 10 at the same time, there is further provided an internal controller C₁ in each electrical stimulation device 10. The internal controller C₁₁ controls a switch 17 which interrupts and closes, respectively, an electrical connection between the associated wireless energy receiver R and the electrode or electrodes 11 of the respective electrical stimulation device 10. Thus, the wireless energy transmitter T is adapted to not only transmit energy but also data to the wireless energy receiver R, which is likewise adapted not only to receive energy wirelessly but also data. For this purpose, an RFID technology is particularly suitable, because an RFID signal may be used to transport both energy and information, as is well known in the art. Accordingly, the data received by the internal controller C₁₁ wirelessly through the wireless energy receiver R includes information about when to close and/or open the switch 17 so that energy may be transferred to the corresponding electrical stimulation device 10 through the wireless energy receiver R. The transfer of energy, on the one hand, and data, on the other hand, between the wireless energy transmitters T and receivers R is indicated by respectively different arrows in FIGS. 3A and 3B.

More specifically, the internal controller C₁₁ of each of the plurality of electrical stimulation devices 10 may be addressed individually by the external controller C₁₁ (or by a remote controller as will be described hereinafter) using an individual code which is specific to the respective internal controller C₁₁. In the situation as discussed above, where the electrical stimulation devices are to be actuated sequentially in order to stimulate the intestine in a wave-like manner, the respective electrical stimulation device may be addressed individually using the individual code of the corresponding internal controller C₁₁. This way, only the electrical stimulation device 10 with the specifically addressed internal controller C₁₁ may be activated by closing the associated switch 17 so that only this particular electrical stimulation device 10 receives electric energy through the wireless energy transmitter T for stimulating the respective section of the intestine 100. Accordingly, the wireless energy transmitter T may comprise a single primary coil extending over the entirety of the secondary coils in the wireless energy receivers R of all of the electrical stimulation devices 10.

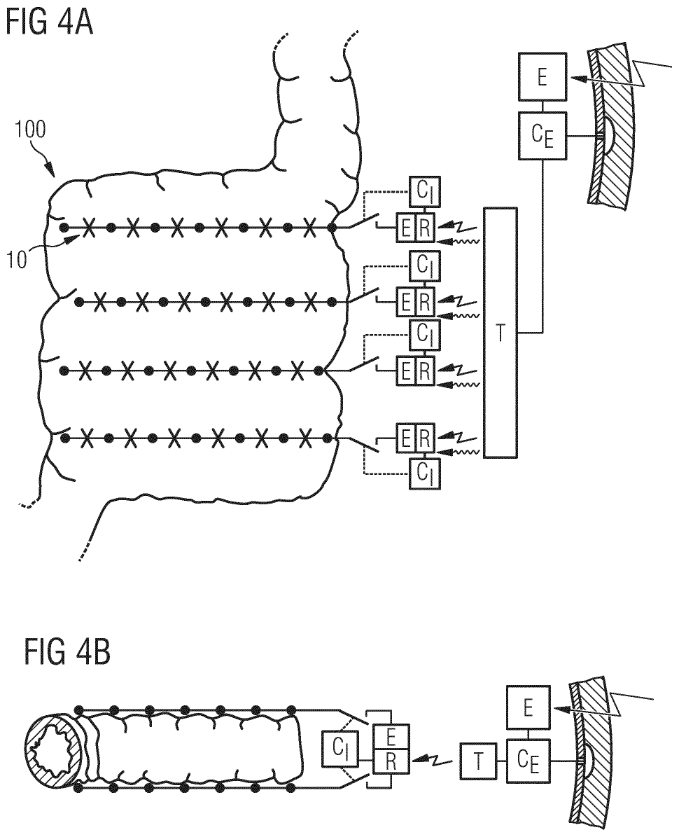

FIGS. 4A and 4B show a fourth embodiment of a system for electrically stimulating tissue of patient's intestine in a top view and a side view, respectively. This embodiment differs from the third embodiment only in that each of the electrical stimulation devices 10 comprises its own energy storage unit E, which may be e.g. a rechargeable battery or a capacitor. Accordingly, the energy storage unit E of the electrical stimulation devices 10 may accumulate energy over time so that sufficient energy is available at each of the electrical stimulation devices 10 when the internal controller C₁₁ receives instructions from the external controller C₁₁ through the (common) wireless energy transmitter T to close and, thus, activate the associated stimulation device 10. While the energy storage unit E connected to the external controller C₁₁ is preferably a rechargeable battery which can store a large amount of energy for a long period of time, the energy storage units E of the electrical stimulation devices 10 are preferably constituted as capacitors, which are substantially smaller, almost neglectable, in size and which may store less but sufficient energy for a shorter but sufficiently long period of time which is needed for the process of stimulating the intestine 100.

Of course, the wireless energy receiver R in the first and second embodiments shown in FIGS. 1A, 1B and 2A and 2B can likewise be replaced with a combination of a wireless energy receiver R, an internal controller C₁₁ and an energy storage unit E, preferably in the form of a capacitor.

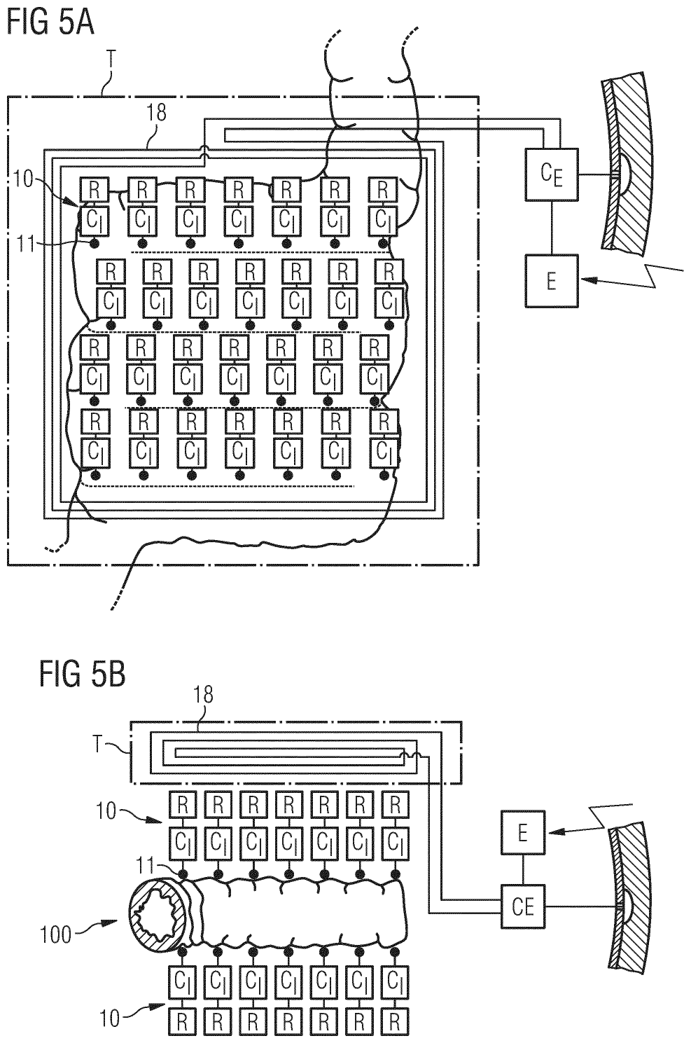

FIGS. 5A and 5B show a fifth embodiment of a system for electrically stimulating tissue of a patient's intestine in a top view and a side view, respectively. This embodiment combines the second embodiment shown in FIGS. 2A, 2B and the third embodiment shown in FIGS. 3A, 3B in that, on the one hand, each electrical stimulation device 10 comprises a single electrode 11 (as in the second embodiment) and, on the other hand, a single wireless energy transmitter T is provided to supply energy to all electrical stimulation devices 10 (as in the third embodiment). Accordingly, the electrical stimulation devices 10 each include an internal controller C₁₁ in addition to a wireless energy receiver R, and the internal controllers C₁₁ may be individually addressed in order to actuate a switch (similar to the switch 17 in FIGS. 3A, 3B, but not shown in FIGS. 5A, 5B) so that stimulation of the intestine 100 may be achieved by means of the corresponding electrical stimulation device 10.

The primary coil 18 of the wireless energy transmitter T is shown in FIGS. 5A and 5B as overlying the plurality of electrical stimulation devices 10 from the top. Since energy transfer from the primary coil 18 to the electrical stimulation devices 10 on the lower side of the intestine 100 is less efficient than the energy transfer to the electrical stimulation devices 10 on the upper side of the intestine 100, a preferred embodiment comprises two wireless energy transmitters T with a primary coil 18A and 18B, respectively, one overlying the electrical stimulation devices 10 on the top side of the intestine 100 and the other one underlying the electrical stimulation devices 10 on the lower side of the intestine 100, as shown in FIG. 5C. Thus, in the embodiment shown in FIG. 5C, each of the wireless energy transmitters T transmits energy to a plurality of (but not all of) the electrical stimulation devices 10.

Of course, the electrical stimulation devices 10 in the embodiments of FIGS. 5A to 5C may further include an energy storage unit E in the same way as described above in relation to the fourth embodiment shown in FIGS. 4A, 4B.

In the embodiments described above, energy is transmitted wirelessly to the energy storage unit E connected to the external controller C, and the external controller C₁₁ is actuated by means of the switch 14, such as a press button. However, as already mentioned before and as shown in FIG. 6 representing a sixth embodiment, a port 15 may be provided outside the patient's body, e.g. mounted to the patient's skin 200, by which an external energy source may be connected to recharge the energy storage unit E, and/or the switch, e.g. press button, may be arranged outside the patient's body, such as on the patient's skin 200. Alternatively, the energy storage unit E may be omitted and a separate energy source may be mounted to the port 15 either permanently or each time when electrical stimulation of the intestine 100 by means of the system is desired.

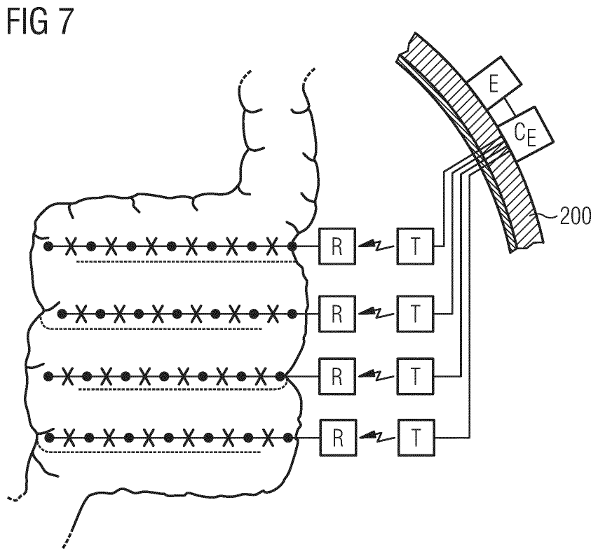

Further alternatively, as shown in FIG. 7 representing a seventh embodiment, the energy storage unit E may be mounted to the patient's skin 200, which preferably is a rechargeable or at least a replaceable battery, but which may likewise be any other kind of energy storage unit. The embodiment shown in FIG. 7 further differs from the previous embodiments in that the external controller C₁₁ is not implanted but located outside the patient's body, here mounted on the patient's skin 200. While the embodiment shown in FIG. 7 has the most features in common with the first embodiment described above in relation to FIGS. 1A, 1B, it is clear that also the other embodiments may be equipped with an external controller C₁₁ and/or an energy storage unit E connected to the external controller C₁₁ which is/are arranged outside the patient's body, preferably on the patient's skin.

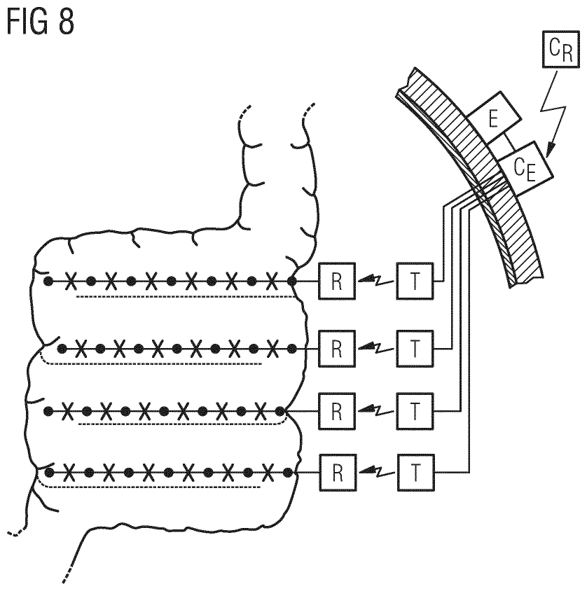

FIG. 8 shows an eighth embodiment which differs from the seventh embodiment in that it comprises a further external controller in the form of a remote controller C_{R} which allows the patient or care person to wirelessly communicate with the external controller C₁₁. Of course, the wireless external controller C₁₁ may likewise be an implanted external controller C₁₁ as shown in the above first to sixth embodiments. This may even be preferable for the patient. For instance, the remote controller C_{R} may make part of a program or app on a remote device, such as a mobile phone, a wristwatch or a different handheld device, which makes the application of the system very convenient for the user and care person.

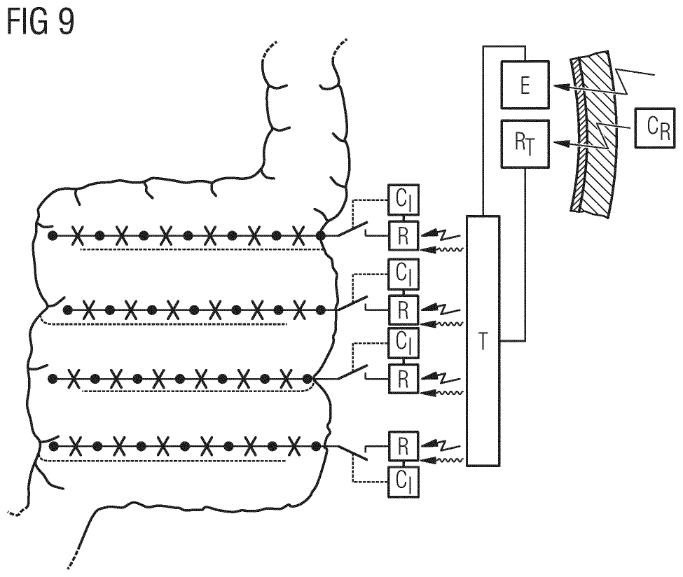

In a preferred ninth embodiment, the external controller C₁₁, either implanted or mounted to the patient's skin, is omitted and it is the wireless remote controller C_{R} which controls the implanted wireless energy transmitter T, as is shown in FIG. 9. Since the remote controller C_{R} communicates wirelessly, an additional wireless energy receiver RT is provided for implantation to receive control signals from the wireless remote controller C_{R} and transmit those signals to the wireless energy transmitter T. The additional wireless energy receiver Ry and the wireless energy transmitter T may be combined to form a transceiver, where applicable. Other than this, the functioning of the tenth embodiment in FIG. 9 is identical to what is described above in relation to the other embodiments. In particular, although the specific embodiment as shown in FIG. 9 comprises electrical stimulation devices 10 which include both a wireless energy receiver R and an internal controller C₁, the electrical stimulation devices 10 may additionally comprise an energy storage unit E or may solely comprise a wireless energy receiver R without an internal controller C₁. In the case that the electrical stimulation devices 10 do not include an internal controller C₁, the system would need to include a wireless energy transmitter T for each of the electrical stimulation devices 10, as in the first embodiment shown in FIGS. 1A and 1C, and an additional wireless energy receiver RT would need to be provided for each of the wireless energy transmitters T so that the electrical stimulation devices 10 can be individually addressed by the remote controller C_{R}.

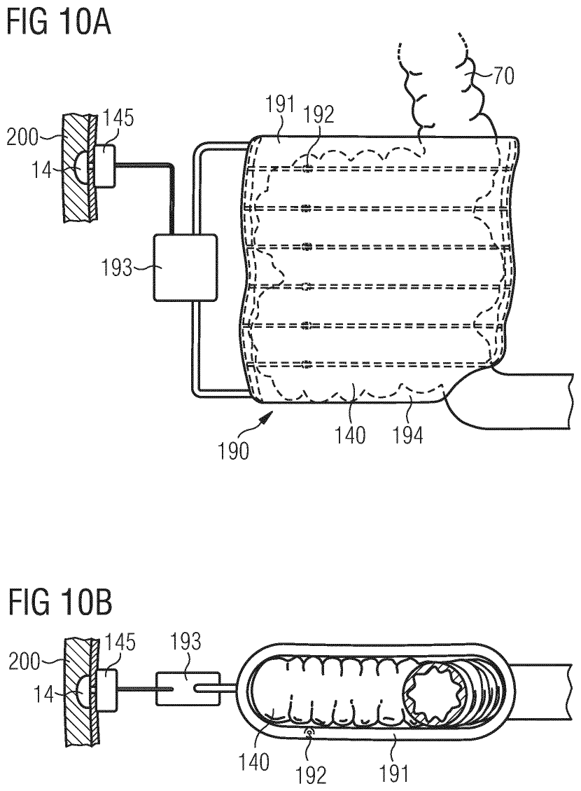

The systems for electrically stimulating tissue of a patient's intestine as described above may be combined with a mechanical or hydraulic constriction device as disclosed in WO 2011/128124 A1. Particularly suitable is the hydraulic constriction device as shown in FIGS. 10A and 10B or any other hydraulic constriction device because hydraulic forces can be easily controlled in a patient. Such constriction devices may act on the same part of the patient's intestine as the electrical stimulation devices. Particularly, such constriction devices may form part of a pump that is configured to advance intestinal contents through the patient's intestine in a downstream direction, and in this context it may cooperate with the electrical stimulation devices 10.

In this context. FIGS. 10A, 10B show as a tenth embodiment the hydraulic type pump comprising a hydraulically acting member 190 adapted to act on the intestinal wall of a reservoir 140 from the outside thereof, the reservoir 140 corresponding to the intestine 100 as shown in the previous embodiments. The hydraulically acting member 190 is connected to an artificial reservoir 193 supplying the hydraulically acting member 190 with hydraulic fluid. The artificial reservoir 193 is of a size sufficiently large to accommodate hydraulic fluid in an amount corresponding to the volume of the intestinal reservoir 140. The artificial reservoir 193 has a flexible wall to allow the hydraulic fluid to be drawn off from and to be filled back into the artificial reservoir 193. The hydraulically acting member 190 is of flexible material and may be tube-like or bag-like so as to accommodate therein the intestinal reservoir 140 including the electrical stimulation devices 10 (not shown). As shown in FIG. 5B, the reservoir 140 is surrounded by the hydraulically acting member 190. The hydraulically acting member 190 is divided into a plurality of chambers, wherein a first chamber 191 and a last chamber 194 are connected to the artificial reservoir 193 by hydraulic conduits. The chambers are interconnected via connections 192, which may be simple holes acting as a throttle or may include one or more valves that are preferably automatically controlled.

Upon activation of the system by the patient using the subcutaneous actuator 14, emptying of the intestinal reservoir 140 is started by supplying hydraulic fluid from the artificial reservoir 193 to the first chamber 191. The next following chambers are supplied with the hydraulic fluid through the connections 192, thereby causing the hydraulically acting member 190 to be filled slowly from the first chamber 191 to the last chamber 194. The filling of the chambers occurs sequentially, with the next following chamber starting to fill before the previous chamber is filled completely. In this manner, intestinal contents are hydraulically squeezed out in the direction towards the exit of the reservoir 140. When the hydraulically acting member 190 is completely filled with hydraulic fluid, the reservoir 140 is completely constricted. The hydraulic fluid is then withdrawn from the chambers of the hydraulically acting member 190 back into the artificial reservoir 193 using negative pressure. The intestinal reservoir 140 may then start to fill up with intestinal contents again.

This process is controlled by the device 150, which is connected to the artificial reservoir 193. Connected to or integrally formed with the artificial reservoir 193 is an electrically driven pump (not shown) for pumping the hydraulic fluid into and withdrawing the hydraulic fluid from the hydraulically acting member. The electrically driven pump is supplied with energy from the combined energy storage means and control device 145. The combined energy storage means and control device 145 may further include the external controller CE and energy storage unit E mentioned above in relation to the electrical stimulation type system. Also, a wireless remote controller C_{R} may be provided as described above.

In another embodiment, each chamber of the hydraulically acting member 190 may have a separate fluid connection to the artificial reservoir 193 in order to be able to be filled individually. The intestinal reservoir 140 may be emptied by consecutively filling two adjacent chambers of the hydraulically acting member 190, i.e. first filling the first and second chamber, then emptying the first chamber while filling the third chamber, then emptying the second chamber while filling the fourth chamber, and so forth. In this manner intestinal contents are squeezed towards and out of the exit of the intestinal reservoir 140.

Alternatively, instead of applying a negative pressure for evacuating the chambers, at least one valve, preferably two valves, may be provided (not shown) between the hydraulically acting member 190 and the artificial reservoir 193 which, when in an appropriate operational position, allows the hydraulic fluid to passively flow from the hydraulically acting member back into the artificial reservoir 193 when the intestinal reservoir 140 fills with intestinal contents and which, when in an appropriate other position, prevents the hydraulic fluid to flow from the hydraulically acting member back into the artificial reservoir when the intestinal reservoir is being emptied.

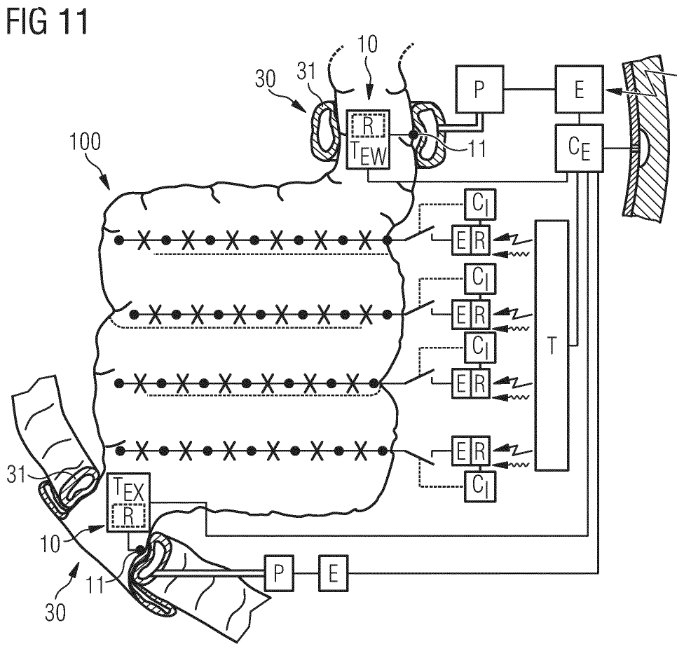

The wirelessly controllable electrical stimulation devices 10 as described above may likewise be implemented in valves for temporarily restricting or even closing an intestinal passageway with or without an additional constriction device, such as a hydraulic constriction device. In other words, a system as described above including a wirelessly controllable electrical stimulation device may be used in a valve, such as an artificial sphincter. Such valve or artificial sphincter may be used as an exit valve and/or as an entry valve of an intestinal reservoir, such as the reservoir made from the patient's intestine as described above or an artificial reservoir. This is further described in relation to an eleventh embodiment of a system for electrically stimulating tissue of the patient's intestine as shown in top view of FIG. 11. This embodiment differs from the third embodiment shown in FIG. 3A in that an exit valve 40 about the patient's colon or rectum or next to a stoma and an entry valve 30 upstream thereof are provided. However, such exit valve 40 and/or entry valve 30 may likewise be provided in any of the other embodiments described herein. While both the exit valve 40 and the entry valve 30 are shown in an open configuration in FIG. 11, usually one of the two valves is closed while the other one is open. The respectively closed state can be achieved by electrically stimulating the neural or muscle tissue of the intestine 100 adjacent the electrode 11 of the corresponding electrical stimulation device 10. While the electrical stimulation devices 10 of the exit and entry valves 40, 30 are shown to be provided with a single electrode 11, they may comprise more than one, such as two, three or four electrodes, preferably on mutually opposing sides of the respective intestinal section. The electrical stimulation device 10 of the exit valve 40 comprises a wireless energy transmitter T_{EX} overlying the wireless energy receiver R of that electrical stimulation device 10, whereas the electrical stimulation device 10 of the entry valve 30 comprises a wireless energy transmitter T_{EN} overlying the associated wireless energy receiver R. Both the wireless energy transmitters T_{EX} and T_{EN} are controlled by the external controller C_{E} in the same way as described before. Thus, stimulation of the respective sections of the intestine 100 may be achieved by transmitting energy from the energy storage unit E to the wireless energy receiver R via the wireless energy transmitters T_{EX} and T_{EN}, respectively, using the external controller C_{E}.

In addition to the electrical stimulation device 10, both the exit valve 40 and entry valve 30 may (or may not) comprise a hydraulic constriction device which may likewise be controlled by the external controller C_{E} so as to coordinate electrical stimulation with hydraulic constriction. The hydraulic constriction device comprises a hollow hydraulic member 41 and 31, respectively, a hydraulic pump P and an energy storage unit E, which may be the same energy storage unit which supplies energy to the electrical stimulation devices 10. In particular, a single energy storage device E may be provided for the entire system. The hydraulic pump P is configured to pump a hydraulic fluid into and withdraw the hydraulic fluid from the interior of the hollow hydraulic members 41 and 31, respectively. FIG. 11 shows the state where the hydraulic fluid is withdrawn from the hollow hydraulic members 31, 41, in which case the respective intestinal section is not constricted. When the hollow hydraulic member is filled with the hydraulic fluid, the intestinal section will be constricted (not shown) to an extent that intestinal contents are prevented from passing through. Of course, instead of a hydraulic constriction device, the exit valve 40 and/or entry valve 30 may comprise a mechanical constriction device serving the same purpose.

In those embodiments of the present disclosure where the system comprises a mechanical or hydraulic constriction device and where the system is configured to electrically stimulate, by means of one or more electrodes, the muscle or neural tissue in an area of the intestine constricted by the mechanical or hydraulic constriction device, such electrical stimulation may be limited to merely increase the blood flow through the tissue of the intestine without causing the intestine to contract or, if at all, contract only partly without completely restricting flow through the respective intestinal section. The purpose thereof is to exercise the tissue wall which is in contact with the constriction device, may it be mechanical or hydraulic. That is, the body tends to react to medical implants, partly because the implant is a foreign object, and partly because the implant interacts mechanically with tissue of the body. Exposing tissue to long-term engagement with, or pressure from, a mechanical or hydraulic or other type of constriction device may deprive the tissue cells of oxygen and nutrients which may lead to deterioration of the tissue, atrophy and eventually necrosis. This may result in migration of the device, including migration through the tissue wall. Exercising the tissue cells by stimulating blood flow increases the tolerance of the tissue for pressure from the implant. As stated, it is preferable to configure the system such that electrical stimulation of the muscle or neural tissue for increasing the blood flow through the tissue of the intestine is adjustable at a low level which is not enough to constrict the intestine.

### Method of Implantation

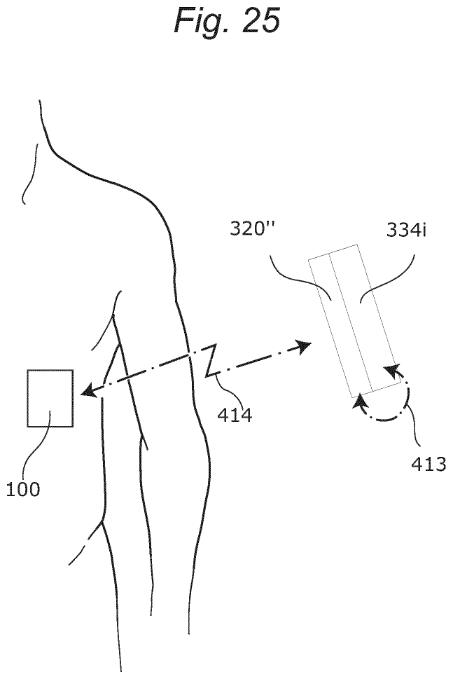

FIG. 19 is a flow chart of a method of implantation of the system comprising the steps of:
making an incision in the body of the patient for accessing the intestine,
inserting one or more electrical stimulation devices, wherein each of the electrical stimulation devices comprises one or more electrodes for electrically stimulating muscle or neural tissue of the intestine and a wireless energy receiver configured to receive energy for stimulating the muscle or neural tissue wirelessly,
placing the electrodes of the electrical stimulation devices in connection with the intestine, which may include implanting at least the electrodes of the electrical stimulation devices in surgically created folds of the patient's intestine,
inserting one or a plurality of wireless energy transmitters,
placing the wireless energy transmitters in proximity to the electrical stimulation devices so as to allow transfer of energy from the energy transmitters to all of the electrical stimulation devices, which may comprise inserting an individual wireless energy transmitter for each one of the electrical stimulation devices so as to allow transfer of energy to the respective one of the electrical stimulation devices,
where the electrical stimulation devices comprise an internal controller, optionally implanting an external controller remote from the internal controller, the external and internal controllers being configured to communicate wirelessly,
optionally, implanting at least one mechanical or hydraulic constriction device outside the patient's intestine in close proximity thereto for constricting the intestine from the outside thereof.

In the step of placing the electrodes of the electrical stimulation devices in connection with the intestine, at least two of the electrodes of an electrical stimulation device are arranged on opposing sides of the patient's intestine.

### Electrical Stimulation/Electrodes

The arrangement of the electrodes as described hereinafter may be implemented in any of the embodiments of the present disclosure, in particular also for the purpose of exercising a tissue wall of the intestine which is in contact with a constriction device, such as a mechanical or hydraulic constriction device. The human and animal body tends to react to a medical implant, partly because the implant is a foreign object, and partly because the implant interacts mechanically with tissue of the body. Exposing tissue to long-term engagement with, or pressure from, an implant may deprive the cells of oxygen and nutrients, which may lead to deterioration of the tissue, atrophy and eventually necrosis. As mentioned, this may result in migration of the device, including migration through the tissue wall. The interaction between the implant and the tissue may also result in fibrosis, in which the implant becomes at least partially encapsulated in fibrous tissue. It is therefore desirable to stimulate or exercise the cells to stimulate blood flow and increase tolerance of the tissue for pressure from the implant.

Muscle tissue is generally formed of muscle cells that are joined together in tissue that can either be striated or smooth, depending on the presence or absence, respectively, of organized, regularly repeated arrangements of myofibrillar contractile proteins called myofilaments. Striated muscle tissue is further classified as either skeletal or cardiac muscle tissue. Skeletal muscle tissue is typically subject to conscious control and anchored by tendons to bone. Cardiac muscle tissue is typically found in the heart and not subject to voluntary control. A third type of muscle tissue is the so-called smooth muscle tissue, which is typically neither striated in structure nor under voluntary control. Smooth muscle tissue makes up the muscular part of the walls of the digestive tract and ducts, including the intestinal tract.

The contraction of the muscle tissue may be activated both through the interaction of the nervous system as well as by hormones. The different muscle tissue types may vary in their response to neurotransmitters and endocrine substances depending on muscle type and the exact location of the muscle.

A nerve is an enclosed bundle of nerve fibers called axons, which are extensions of individual nerve cells or neurons. The axons are electrically excitable, due to maintenance of voltage gradients across their membranes, and provide a common pathway for the electrochemical nerve impulses called action potentials. An action potential is an all-or-nothing electrochemical pulse generated by the axon if the voltage across the membrane changes by a large enough amount over a short interval. The action potentials travel from one neuron to another by crossing a synapse, where the message is converted from electrical to chemical and then back to electrical.

The distal terminations of an axon are called axon terminals and comprise synaptic vesicles storing neurotransmitters. The axonal terminals are specialized to release the neurotransmitters into an interface or junction between the axon and the muscle cell. The released neurotransmitter binds to a receptor on the cell membrane of the muscle cell for a short period of time before it is dissociated and hydrolyzed by an enzyme located in the synapse. This enzyme quickly reduces the stimulus to the muscle, which allows the degree and timing of muscular contraction to be regulated carefully.

The action potential in a normal skeletal muscle cell is similar to the action potential in neurons and is typically about-90 mV. Upon activation, the intrinsic sodium/potassium channel of the cell membrane is opened, causing sodium to rush in and potassium to trickle out. As a result, the cell membrane reverses polarity and its voltage quickly jumps from the resting membrane potential of −90 mV to as high as +75 mV as sodium enters. The muscle action potential lasts roughly 2 to 4 ms, the absolute refractory period is roughly 1 to 3 ms, and the conduction velocity along the muscle is roughly 5 m/s. This change in polarity causes in turn the muscle cell to contract.

The contractile activity of smooth muscle cells is typically influenced by multiple inputs such as spontaneous electrical activity, neural and hormonal inputs, local changes in chemical composition, and stretch. This in contrast to the contractile activity of skeletal and cardiac muscle cells, which may rely on a single neural input. Some types of smooth muscle cells are able to generate their own action potentials spontaneously, which usually occurs following a pacemaker potential or a slow wave potential. However, the rate and strength of the contractions can be modulated by external input from the autonomic nervous system. Autonomic neurons may comprise a series of axon-like swellings, called varicosities, forming motor units through the smooth muscle tissue. The varicosities comprise vesicles with neurotransmitters for transmitting the signal to the muscle cell.

The muscle cells described above, i.e., the cardiac, skeletal, and smooth muscle cells are known to react to external stimuli, such as electrical stimuli applied by electrodes. A distinction can be made between stimulation transmitted by a nerve and direct electrical stimulation of the muscle tissue. In case of stimulation via a nerve, an electrical signal may be provided to the nerve at a location distant from the actual muscle tissue, or at the muscle tissue, depending on the accessibility and extension of the nerve in the body. In case of direct stimulation of the muscle tissue, the electrical signal may be provided to the muscle cells by an electrode arranged in direct or close contact with the cells. However, other tissue such as fibrous tissue and nerves may of course be present at the interface between the electrode and the muscle tissue, which may result in the other tissue being subject to the electrical stimulation as well.

In the context of the present application, the electrical stimulation discussed in connection with the various aspects and embodiments may be provided to the tissue in direct or indirect contact with the implantable constriction device. Preferably, the electrical stimulation is provided by one or several electrode elements arranged on or in the tissue or at the interface or contact surface between an implantable constriction device and the tissue. Thus, the electrical stimulation may, in terms of the present disclosure, be considered as a direct stimulation of the tissue. Particularly when contrasted to stimulation transmitted over a distance by a nerve, which may be referred to as an indirect stimulation or nerve stimulation.

Hence, an electrode arrangement comprising one or several electrode elements may be arranged in, partly in, on, or in close vicinity of the tissue that is to be exercised by means of an electrical signal. Preferably, the electrode may be arranged to transmit the electrical signal to the portions of the tissue that is to be stimulated so as to cause it to constrict or so as to cause it to exercise with no or little constriction, namely in situations where the tissue is affected, or risks to be affected, by mechanical forces exerted by a medical implant. Thus, the electrode element may be considered to be arranged between the medical implant, such as a constriction device, and the tissue against which the implant is arranged to rest, when implanted.

During operation of the electrical stimulation device, the electrical signal may cause the muscle cells to contract and relax repeatedly. If such activity is little, this action of the cells may be referred to as exercise and may have a positive impact in terms of preventing deterioration and damage of the tissue. Further, the exercise may help to increase tolerance of the tissue for pressure and mechanical forces generated by the medical implant.

The interaction between the electrode or electrodes of the electrical stimulation device and the tissue of the patient's intestine is to a large extent determined by the properties at the junction between the tissue and the electrode element. The active electrically conducting surface of the electrode element (in the following referred to as “metal”, even though other materials are equally conceivable) can either be uncoated resulting in a metal-tissue interface or insulated with some type of dielectric material. The uncoated metal surface of the electrode may also be referred to as a bare electrode. The interface between the electrode and the tissue may influence the behavior of the electrode since the electrical interaction with the tissue is transmitted via this interface. In the biological medium surrounding the electrode, such as the actual tissue and any electrolyte that may be present in the junction, the current is carried by charged ions, while in the material of the electrode the current is carried by electrons. Thus, in order for a continuous current to flow, there needs to be some type of mechanism to transfer charge between these two carriers.

In some examples, the electrode may be a bare electrode wherein the metal may be exposed to the surrounding biological medium when implanted in, or at, the muscle or neural tissue that is to be stimulated. In this case there may be a charge transfer at a metal-electrolyte interface between the electrode and the tissue. Due to the natural strive for thermodynamic equilibrium between the metal and the electrolyte, a voltage may be established across the interface which in turn may cause an attraction and ordering of ions from the electrolyte. This layer of charged ions at the metal surface may be referred to as a “double layer” and may physically account for some of the electrode capacitance.

Hence, both capacitive faradaic processes may take place at the electrode. In a faradaic process, a transfer of charged particles across the metal-electrolyte interface may be considered as the predominant current transfer mechanism. Thus, in a faradaic process, after applying a constant current, the electrode charge, voltage and composition tend to go to constant values. Instead, in a capacitive (non-faradaic) process, charge is progressively stored at the metal surface and the current transfer is generally limited to the amount which can be passed by charging the interface.

In some examples, the electrode may comprise a bare electrode portion, i.e. an electrode having an uncoated surface portion facing the tissue such that a conductor-tissue interface is provided between the electrode and the tissue when the electrode element is implanted. This allows for the electrical signal to be transmitted to the tissue by means of a predominantly faradaic charge transfer process. A bare electrode may be advantageous from a power consumption perspective since a faradaic process tends to be more efficient than a capacitive-charge transfer process. Hence, a bare electrode may be used to increase the current transferred to the tissue for a given power consumption.

In some examples, the electrode may comprise a portion that is at least partly covered by a dielectric material so as to form a dielectric-tissue interface with the muscle tissue when the electrode is implanted. This type of electrode allows for a predominantly capacitive, or non-faradaic, transfer of the electrical signal to the muscle tissue. This may be advantageous over the predominantly faradaic process associated with bare electrodes since faradaic charge transfer may be associated with several problems. Examples of problems associated with faradaic charge transfer include undesirable chemical reactions such as metal oxidation, electrolysis of water, oxidation of saline, and oxidation of organics. Electrolysis of water may be damaging since it produces gases. Oxidation of saline can produce many different compounds, some of which are toxic. Oxidation of the metal may release metal ions and salts into the tissue which may be dangerous. Finally, oxidation of organics in a situation with an electrode element directly stimulating tissue may generate chemical products that are toxic.

These problems may be alleviated if the charge transfer by faradaic mechanisms is reduced, which may be achieved by using an electrode at least partly covered by a dielectric material. Preferably, the dielectric material is chosen to have as high capacitance as possible, restricting the currents flowing through the interface to a predominantly capacitive nature.

Several types of electrode elements can be combined with the present disclosure. The electrode element can for example be a plate electrode, comprising a plate-shaped active part forming the interface with the tissue. In other examples, the electrode may be a wire electrode, formed of a conducting wire that can be brought in electrical contact with the tissue. Further examples may include needle-or pin-shaped electrodes, having a point at the end which can be attached to or inserted in the muscle tissue. The electrodes may for example be encased in epoxy for electrical isolation and protection, and comprise gold wires or contact pads for contacting the muscle tissue.

It will be appreciated that both faradaic and capacitive mechanisms may be present at the same time, irrespective of the type of electrode used. Thus, capacitive charge transfer may be present also for a bare electrode forming a metal-tissue interface, and faradaic charge transfer may be present also for a coated electrode forming a dielectric-tissue interface. It has been found that the faradaic portion of the current delivered to the muscle tissue can be reduced or even eliminated by reducing the duration of the pulses of the electrical signal. Reducing the pulse duration has turned out to be an efficient way of increasing the portion of the signal which can be passed through the interface as a capacitive current, rather than by a faradaic current. As a result, shorter pulses may produce less electrode and tissue damage.

The capacitive portion of the current may further be increased, relative to the faradaic portion, by reducing the amplitude of the current pulses of the electrical signal. Reducing the amplitude may reduce or suppress the chemical reactions at the interface between the electrode and the tissue, thereby reducing potential damage that may be caused by compounds and ions generated by such reactions.

In one example, the electrical stimulation may be controlled in such a manner that a positive pulse of the electrical signal is followed by a negative pulse (or, put differently, a pulse of a first polarity being followed by a pulse of a second, reversed polarity), preferably of the same amplitude and/or duration. Advantageously, the subsequent negative (or reversed) pulse may be used to reverse or at least moderate chemical reactions or changes taking place in the interface in response to the first, positive pulse. By generating a reversed pulse, the risk of deterioration of the electrode and/or the tissue at the interface between the electrode and the muscle tissue may be reduced.

Although FIG. 11 shows an embodiment where the entry and exit valves 30, 40 each comprise a single electrode 11, there may be provided more than one electrode 11 for electrically stimulating the tissue of the intestinal section for exercising the muscle tissue in order to improve the conditions for long-term implantation of the entry and exit valves 30, 40. In the embodiment of FIG. 11, the electrode 11 is arranged underneath the hollow hydraulic members 31, 41 and, thus, placed in abutment and in electrical connection with the tissue of the intestine. Alternatively, a first and possibly even a second electrode 11 may be placed on a first side of the luminary organ, and a third and possibly even a fourth electrode 11 may be placed on a second, opposing side of the intestine. Each of the two or four electrodes 11 are connected to the external controller C_{E} (alternatively a wireless remote controller C_{R}) for controlling the electrical stimulation of the tissue of the intestine such that the tissue of the intestine is stimulated by a series of electrical pulses. The pulses may comprise a pulse of a first polarity followed by a pulse of a second, reversed polarity, and the pulsed electrical stimulation signal generated may comprise a pulse frequency of 0.01-150 Hz. The electrical stimulation signal may comprise a pulse duration of 0.01-100 ms and a pulse amplitude of 1-15 mA. More specifically, the electrical stimulation signal may comprise a pulse frequency of 0.15-0.25 Hz, a pulse duration of 20-30 ms and a pulse amplitude of 3-10 mA. Further, the electrical stimulation signal may comprise a build-up period of 0.01-2 s in which the amplitude gradually increases, a stimulation period of 1-60 s, and a stimulation pause of 0.01-60 s, wherein the electrical signal may comprise a pulse frequency of 1-50 Hz and a pulse duration of 0.1-10 ms.

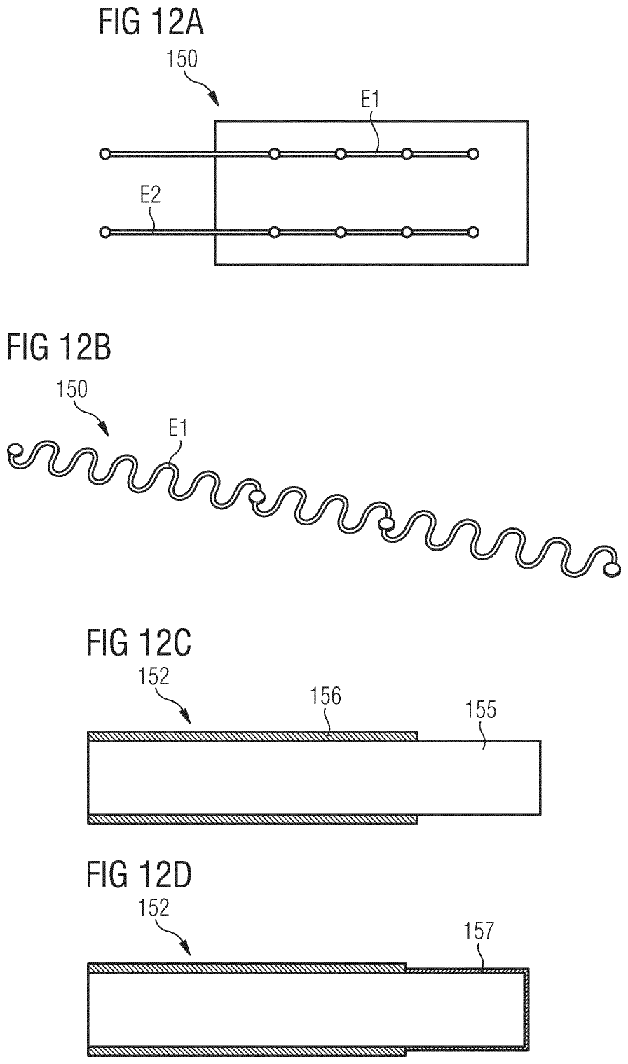

FIG. 12A is an example of a bipolar electrode arrangement 150, comprising a first and a second electrode element 152, 154 which may be similarly configured as the electrode elements discussed with reference to any of the previous embodiments. In the following figures, the first and second electrode elements will be distinguished by reference numerals E1 and E2, respectively. The first and second electrode elements E1, E2 may be connected to different electrical potentials. Thus, the first electrode element E1 can be operated as an anode and the second electrode element E2 can be operated as a cathode. In alternative embodiments, however, both electrode elements E1, E2 may be operated as cathodes, while using the tissue of the body as anode. The electrode elements E1, E2 may be attached directly to an outer surface of the implantable device, such as disclosed with reference to FIG. 11. In some examples the electrode elements E1, E2 may be arranged on a support, such as a flexible patch, which may be configured to be attached to the implantable constriction device 30. The electrode arrangement 150 can be arranged between the implantable constriction device 30 and the tissue (such as disclosed with reference to FIG. 11) and may in some examples be provided as a separate, physically distinct item and in other examples be integrated in the apparatus 100. The electrode arrangement 150 may comprise one or several contact pads for increasing the contact surface between the electrode and the tissue when implanted. During operation, the electrical signal may be delivered to the muscle tissue by means of the first and second electrode elements E1, E2 so as to stimulate contraction of the muscle cells.

FIG. 12B is another example of an electrode arrangement 150, which in the present example may be a unipolar electrode element 152, 154. The electrode element E1 may for example be operated as a cathode when implanted. The electrode element 152 may be formed of a flat, coiled wire for increasing the contact surface between the electrode element 152 and the tissue. Further, the coiled configuration allows for a certain mechanical flexibility of the electrode element 152 such that it can follow the muscle tissue during contraction and relaxation.

FIG. 12C illustrates the end portion of a needle-or pin-shaped electrode arrangement 150, wherein the active portion of the electrode element 152 is provided as a bare electrode surface 155 at the end of the electrode element 152, protruding from an insulation 156 covering the rest of the electrode element 152. Thus, when implanted at or in the muscle tissue, the active, bare electrode surface 155 of the electrode element 152 may form a metal-tissue interface with the muscle tissue, wherein the interface may surround the end portion of the electrode element 152 so as to provide a relatively large contact surface. The present example is advantageous in that it can be inserted into the tissue, thereby allowing for a selective stimulation at a certain depth of the tissue.

FIG. 12D shows a similar electrode element 152 as the one in FIG. 12C, with the difference that the present electrode element 152 comprises an active portion that is covered by a dielectric material 157 so as to protect the electrode material from deterioration and to facilitate capacitive current transfer. The dielectric material 157 may for example be electrochemically deposited tantalum oxide, which allows the electrical charge to pass through the interface but reduces the risk for electrode corrosion, gas formation and metabolite reactions.

It will be appreciated that both faradaic and capacitive mechanisms may be present at the same time, irrespectively of the type of electrode used. Thus, capacitive charge transfer may be present also for a bare electrode forming a metal-tissue interface, and faradaic charge transfer may be present also for a coated electrode forming a dielectric-tissue interface. It has been found that the faradaic portion of the current delivered to the muscle tissue can be reduced or even eliminated by reducing the duration of the pulses of the electric signal. Reducing the pulse duration has turned out to be an efficient way of increasing the portion of the signal which can be passed through the interface as a capacitive current, rather than by a faradaic current. As a result, shorter pulses may produce less electrode and tissue damage.

The capacitive portion of the current may further be increased, relative to the faradaic portion, by reducing the amplitude of the current pulses of the electrical signal. Reducing the amplitude may reduce or suppress the chemical reactions at the interface between the electrode and the tissue, thereby reducing potential damage that may be caused by compounds and ions generated by such reactions. In one example, the electrical stimulation may be controlled in such a manner that a positive pulse of the electrical signal is followed by a negative pulse (or, put differently, a pulse of a first polarity being followed by a pulse of a second, reversed polarity), preferably of the same amplitude and/or duration. Advantageously, the subsequent negative (or reversed) pulse may be used to reverse or at least moderate chemical reactions or changes taking place in the interface in response to the first, positive pulse. By generating a reversed pulse, the risk of deterioration of the electrode and/or the tissue at the interface between the electrode and the muscle tissue may be reduced.

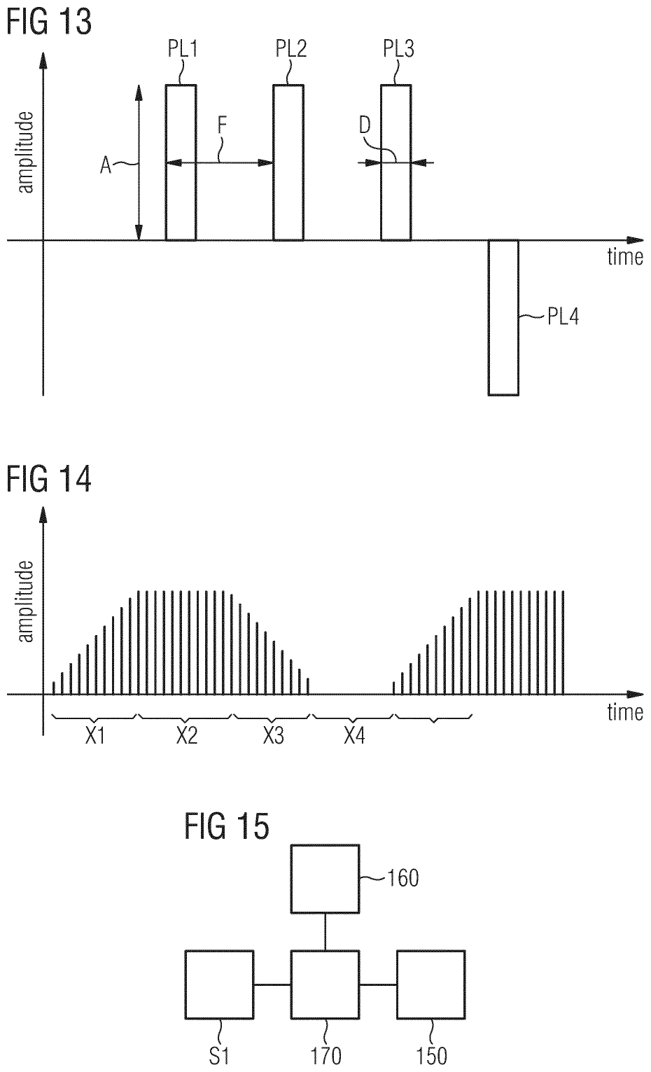

FIG. 13 shows an example of a pulsed electrical signal to be applied to an electrode for electrically stimulating muscle tissue via an electrode-tissue interface as discussed above. The electrical signal may be generated by a stimulation controller arranged outside the body or implanted in the body (as described with reference to FIG. 11). The stimulation controller 170 may be operatively connected to the electrode element 152, 154 by means of a lead 172, and the electrical signal shown in the present figure may either reflect the signal as generated at the stimulation controller 170, or the signal as delivered to the electrode element 152, 154 at the electrode-tissue interface. The characteristics of the electrical signal may be selected and varied determined on the electrical and properties at the electrode-tissue interface and on the actual response of the tissue. The electrical stimulation delivered to the muscle cells may depend on several factors, such as the configuration and placement of the electrode element 152, 154 at the tissue, the presence of fibrous material at the interface, the composition of the electrolyte in the interface, accumulation of non-conducting material on the electrode surfaces, etcetera. It is therefore suggested that the characteristics of the electric signal, as shown in the present figure, be selected, and varied based on an observed or estimated response from the stimulated tissue.

In the present example, the electrical signal is a pulsed signal comprising square waves PL1, PL2, PL3, PL4. However, other shapes of the pulses may be employed as well. The pulse signal may be periodic, as shown, or may be intermittent (i.e., multiple series of pulses separated by periods of no pulses). The pulses may have an amplitude A, which may be measured in volts, ampere, or the like. Each of the pulses of the signal may have a pulse width D. Likewise, if the signal is periodic, the pulse signal may have a period F that corresponds to a frequency of the signal. Further, the pulses may be either positive or negative in relation to a reference.

The pulse frequency may for example lie within the range of 0.01-150 Hz. More specifically, the pulse frequency may lie within at least one of the ranges of 0.1-1 Hz, 1-10 Hz, 10-50 Hz and 50-150 Hz. It has been observed that relatively low pulse frequencies may be employed to imitate or enhance the slow wave potential associated with pacemaker cells of the smooth muscle tissue. Thus, it may be advantageous to use relatively low pulse frequencies, such as 0.01-0.1 Hz or frequencies below 1 Hz or a few Hz for such applications.

The pulse duration may for example lie within the range of 0.01-100 milliseconds (ms), such as 0.1-20 milliseconds, and preferably such as 1-5 ms. The natural muscle action potential has in some studies been observed to last about 2-4 ms, so it may be advantageous to use a pulse duration imitating that range.

The amplitude may for example lie within the range of 1-15 milliamperes (mA), such as 0.5-5 mA in which range a particularly good muscle contraction response has been observed in some studies.

In a preferred, specific example the electrical stimulation may hence be performed using a pulsed signal having a pulse frequency of 10 Hz, a pulse duration of 3 ms and an amplitude of 3 mA.

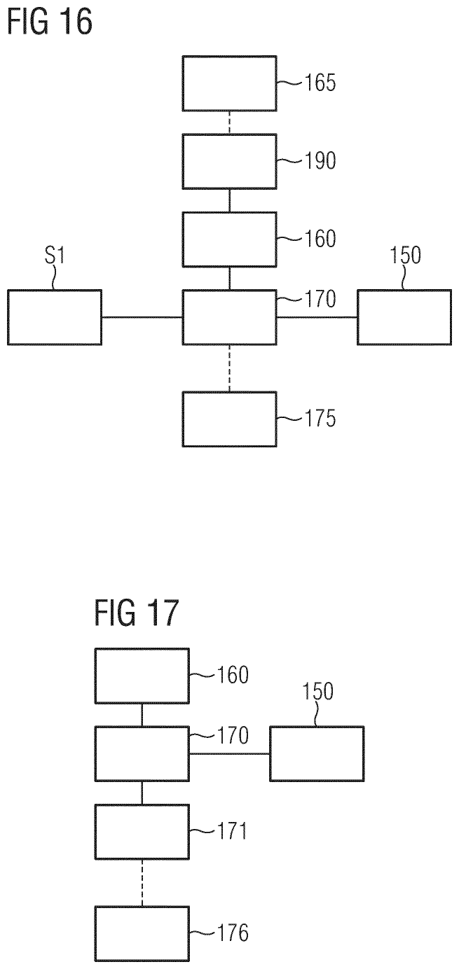

FIG. 14 shows an example of a pulsed signal, comprising build-up period X1, in which the amplitude is gradually increasing, a stimulation period X2 during which the muscle tissue is exposed to a contracting stimulation 30 signal, a ramp down period X3 in which the amplitude is gradually decreasing, and a stimulation pause X4 before a new build-up period is initiated. The build-up period may for example be 0.01-2 seconds, the stimulation period 1-85 seconds, the ramp-down period 0.01-2 seconds, and the stimulation pause 0.01-60 seconds. The pulse frequency may for example be 1-50 Hz, the pulse duration 0.1-10 milliseconds and the amplitude during the stimulation period be 1-15 milliampere. The stimulation of skeletal muscle tissue may for example be performed using a frequency of 50 Hz and pulses having a duration of 100 μs. The current amplitude may be 1, 2.5, 7.5 or 10 mA. In particular, a desired muscle contraction response has been experimentally observed within a range of 0.5 to 5.0 mA. In the present example, a coiled electrode may be used as a cathode. Another example design is a multi-stranded wire arranged in a helical design. They can be imbricated in the muscular wall of the fundus (or esophagus) and can be stimulated in any desired pattern. The stimulus parameters may for example be biphasic pulses, 10 to 40 Hz, lasting 0.1 to 5 ms, with a current density of 3 to 5 mA/cm2.

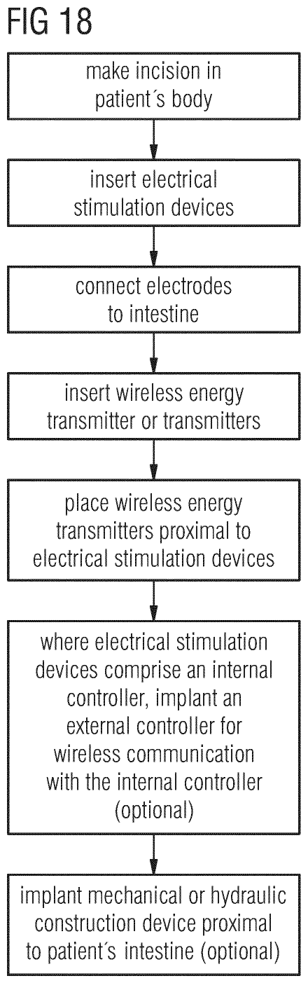

FIG. 15 is a schematic outline of a system for electrically stimulating or exercising muscle cells to increase tolerance of the tissue for pressure from the apparatus 100. The system may be used in combination with the implantable apparatus 100 and may in some examples be comprised in such an apparatus 100. The system may comprise an electrode arrangement 150 which may be similarly configured as the electrodes arrangements/electrode elements discussed above in connection with the previous examples, an energy source 160 for providing the electrical energy required for generating the electrical signal, and a stimulation controller 170 controlling the generation of the electrical signal.

The electrode arrangement 150, which may comprise one or several electrode elements 152, 154, such as a bare electrode or an electrode at least partly covered by a dielectric material 157 shown in FIG. 12D, may be configured to be implanted in the muscle tissue to be stimulated, or to engage the muscle, so as to form an electrode-tissue interface through which the stimulating signal may be transferred. Alternatively, or additionally, the electrode element 152, 154 may be arranged in close vicinity to the muscle tissue such that an electrical coupling between the electrode element and the muscle tissue may be established. This may for example be the case when other tissue, such as connective tissue, is present between the implanted device and the muscle tissue.

The electrode may be electrically connected to the energy source 160, for example by means of a wiring or a lead, such that the electrical signal may be transferred to the electrode-tissue interface. In some examples, the electrode 152, 154 may be integrated with or attached to the apparatus so that the electrode 152, 154 when implanted in the patient is arranged at the interface between the apparatus 100 and the muscle tissue. The electrode 152, 154 can thereby be used for exercising the muscle tissue that is mechanically affected by the implant.

The energy source 160 may for example be of a non-rechargeable type, such as a primary cell, or of a rechargeable type, such as a secondary cell. The energy source 160 may be rechargeable by energy transmitted from outside the body, from an external energy source, or be replaced by surgery. Further, the electrode arrangement 150 may be operably connected to a stimulation controller 170, which may comprise an electrical pulse generator, for generating the electrical pulse. The stimulation controller 170 may be integrated with the energy source 160 or provided as a separate, physically distinct unit which may be configured to be implanted in the body or operate from the outside of the body. In case of the latter, is may be advantageous to allow the external control unit to communicate wirelessly with the stimulation controller 150.

The system may according to some examples comprise a sensor S1 that is configured to sense a physical parameter of the body and/or the apparatus 100. The sensor S1 may for example be employed to sense or detect a bodily response to the electrical stimulation, such as for example a contraction of the stimulated muscle tissue. In an example, the sensor S1 may be configured to sense action potentials that are being sent to the muscle tissue. The action potentials may for example be generated by pacemaker cells of the muscle tissue, which may be registered by the sensor S1 and transmitted to the stimulation controller 170. The stimulation controller 170 may use the received signal when controlling the energy source 160, such that the generated electrical signal amplifies the sensed action potentials.

The energy source 160 may preferably be an implantable energy source 160 configured to be placed on the inside of the patient's body. Preferably, the implantable energy source 160 may comprise a secondary cell, which can be charged from the outside of the body so as to reduce the need for surgical battery replacement procedures. As indicated in the present figure, the implantable energy source 160 may be configured to be supplied with electrical energy from an external energy source 165 arranged outside the body. In such an example, the system may further comprise an implantable charger 190 configured to be electrically connected to the implantable energy source 160 and to enable charging of the implantable energy source 160 by the external energy source 165. The implantable charger 190 may for example be configured to be electrically connected to the implantable energy source 160 by means of a wiring or a lead, such that the electrical energy may be transferred from the implantable charger 190 to the implantable energy source 160. The implantable charger 190 may further be coupled to the external energy source 165 by a wireless coupling or by a wired coupling, using a wiring or lead which may be similar to the one between the charger 190 and the implantable energy source 160. In case of the latter, the wiring or lead may terminate in a terminal which may be access via the skin of the patient, either as a contact port surfacing the skin or being arranged under the skin. Electrical energy may then be transmitted to the charger 190 by connecting the external energy source 165 to the port, for example by incising the skin to expose the port and making it possible for the external energy source 165 to be plugged in.

Alternatively, the implantable charger 190 may be configured to receive energy from the external energy source 165 wirelessly, such as for example inductively. In this case, the charger 190 may comprise an electromagnetic coil configured to receive the electrical power wirelessly from the external energy source 165. The charger 190 may for example be arranged subcutaneously so as to facilitate inductive transfer of the energy via the skin of the patient.

The charging of the implantable energy source 160 may be controlled according to several different schemes. In an example, the charging of the implantable energy source 160 may be controlled by controlling the receipt of electrical power, from the external energy source, at the implantable charger 190. Put differently, the charger 190 may be configured to vary or control its capability of receiving electrical energy from the external energy source 165.

Hence, the amount of electrical power delivered to the implantable energy source 160 may be regulated at the implantable charger 190 rather than at the external energy source 165, which hence may be allowed to transmit a substantially constant power. By varying the receipt at the charger 190, rather than the transmission at the external power source 165, the charging of the implantable energy source 160 may be performed without sending control signals to the external energy source 165. Instead, the intelligence required for regulating and controlling the charging of the implanted energy source 160 may be accommodated within the body of the patient, without the need of communication with the outside of the body.

In an alternative embodiment, the charging of the implantable energy source 160 may be controlled by controlling the transmission of electrical power at the external energy source 165. Thus, the charger 190 (or any other component of the apparatus/system arranged in the body) may send transmission instructions, for example via a control signal, to the external energy source 165 which may regulate its transmitting power accordingly.

The charging of the implantable energy source 160 may be controlled by the controller 170, which hence may be configured to issue control instructions to the implantable charger 190 and/or the external energy source 165, as discussed above. In some examples, the controller 170 may be configured to indicate a functional status of the implantable energy source 160, such as for example charge level, charging capacity, voltage and/or temperature of the implantable energy source 160. The functional status may for example be used for controlling the charging of the implantable energy source 160 as described above, and for indicating the status of the implantable energy source 160 to the patient or another, external entity such as medical staff. The functional status may for example be transmitted to the outside of the body, where it can be interpreted and used for diagnosis of the status/condition of the implanted apparatus. Further, the functional status may be transmitted to the outside of the body to provide a warning signal, for example indicating low battery or overheating. The transmission of a signal to/from the controller 170 is described in further detail in connection with the following FIGS. 15 to 17.

The functional status may for example be based on a signal from a sensor, such as a temperature sensor configured to sense a temperature of the implanted energy source 160, or a current or voltage meter configured to measure an electrical condition of the implanted energy source 160. The sensor output may be transmitted to the controller 170, for example by means of a wiring or electrical conductor, where it can be processed and acted upon in the form of an issued signal comprising control instructions for the charger 190/external energy source 165 and/or functional status information.

The functional status may in some examples be transmitted via a carrier signal to the outside of the body by means of a transmitter, which for example may be arranged subcutaneously. In some example the transmitter may be integrated in the charger 190.

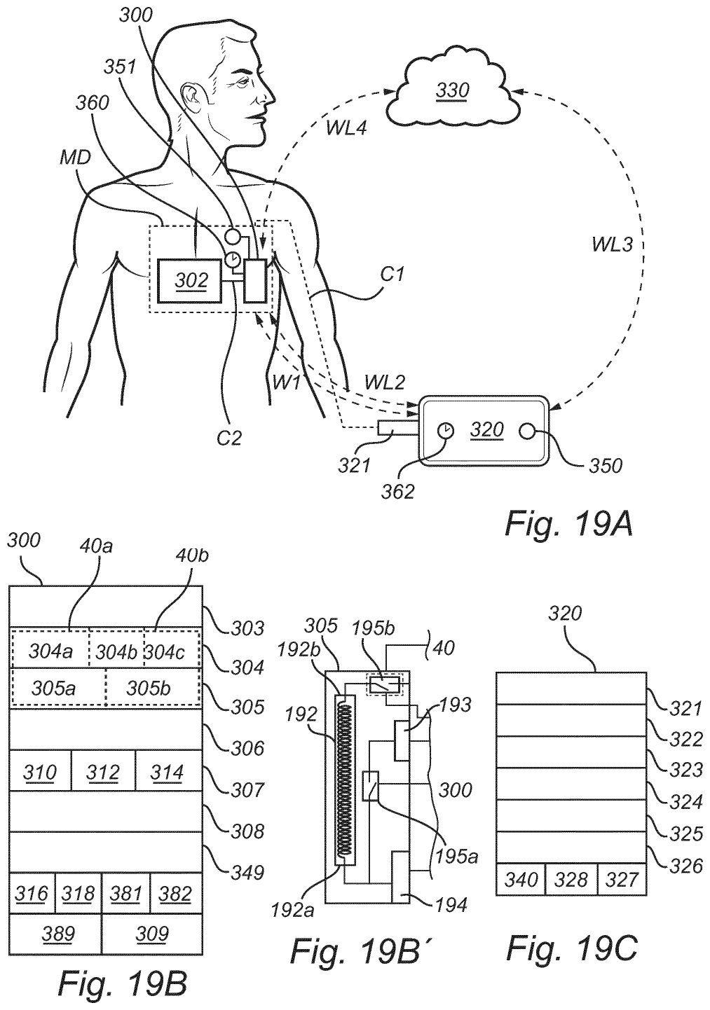

FIG. 16 shows a similar embodiment as the system described above with reference to FIG. 15. However, as indicated in the present figure, the system may further comprise an external signal transmitter 175, such as a wireless remote 175, which may be configured to be operably connected to the controller 170. The external signal transmitter 175 may be arranged to allow for the patient or another external entity, such as a service technician or medical staff, to interact with the controller 170. The external signal transmitter 175 may for example be used to control, or adjust, the operation of the implanted controller 170 in order to affect or adjust the electrical stimulation signal delivered to the tissue by the electrode arrangement 150. The external control of the controller 170 may for example serve the purpose of increasing or reducing an amplitude or frequency of the electrical stimulation signal, or for activating/deactivating the electrical stimulation. In an example, the external signal transmitter 175 may be used for increasing the electrical stimulation of the cardiac sphincter in response to experienced reflux symptoms. In this way, the patient may be allowed to increase the contraction of the cardiac sphincter so as to further hinder stomach contents from rising in the esophagus.

The signal, by which the external signal transmitter 175 is communicating with the implanted controller 170, may be selected from the group consisting of: a sound signal, an ultrasound signal, an electromagnetic signal, and infrared signal, a visible light signal, an ultra violet light signal, a laser signal, a microwave signal, a radio wave signal, an X-ray radiation signal and a gamma radiation signal.

While illustrated as separate components/entities in the figure, it is appreciated that the implanted, or internal, controller 170 may be integrated in the implantable charger 190 and/or in the implantable energy source 160. Further, the external signal transmitter 175 may be integrated in the wireless remote.

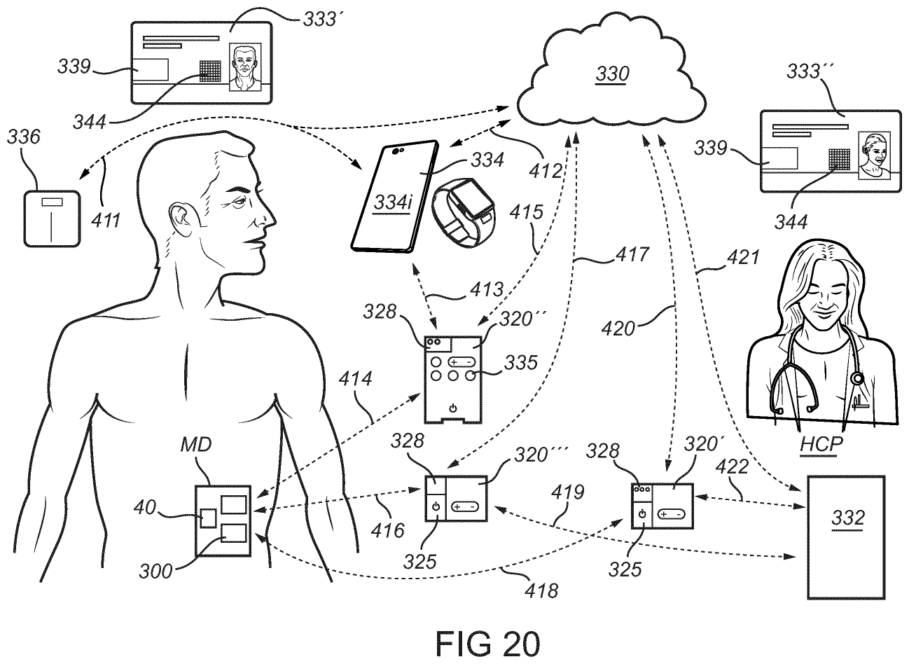

FIG. 17 is a schematic diagram of a system, or an apparatus, which may be similarly configured as the system described with reference to FIGS. 15 and 16. Hence, a system is disclosed, comprising an electrode arrangement 150 for exercising muscle tissue affected by an implanted apparatus according to any of the embodiments discussed above in connection with FIGS. 1 to 11, and a controller 170 configured to be operably connected to the electrode arrangement 150 for controlling the electrical stimulation of the muscle tissue. The controller 170 may be coupled to an implantable energy source 160 for providing the electrode arrangement with electrical power according to a stimulation signal or pattern generated by the controller 170.

FIG. 17 further illustrates an implantable communicator 171, which may be configured to transmitting a signal between the controller 170 and the outside of the patient's body, similar to what is described above in connection with FIG. 16. The communicator 171 may be comprised in the control unit 170 or provided as a separate unit. The communicator 171 may hence be used for transmitting the signal comprising the functional status of the implantable energy source 160, and for communicating with an external controller 176 used for controlling or adjusting the operation of the implantable controller 170. The external controller 176 may for example be comprised in a remote controller 175 as shown in FIG. 16.

The implantable controller 170, which also may be referred to as an internal controller or a stimulation controller 170, may be understood as any implantable unit capable of controlling the electrical stimulation of the tissue. A controller could include an electrical signal generator, a modulator or other electrical circuitry capable of delivering the electrical stimulation signal to the electrode arrangement. Further, the controller may be capable of processing control signals and generate the electrical stimulation signal in response thereto, and further to generate control signals for the control of other components of the system or apparatus, such as for example the implanted energy source 160 and/or the implantable charger 190. A control signal may thus be understood as any signal capable of carrying information and/or electric power such that a component of the system/apparatus can be directly or indirectly controlled.

The controller may comprise a processing unit, such as a CPU, for handling the control of the electrode arrangement 150 and other components of the system. The processing unit could be a single central processing unit or could comprise two or more processing units. The processing unit could comprise a general-purpose microprocessor and/or an instruction set processor and/or related chips sets and/or special purpose microprocessors such as ASICs (Application Specific Integrated Circuit). The processing unit may also comprise memory for storing instruction and/or data. The controller 170 could be adapted to keep track of different stimulation patterns and periods used for the stimulation of the muscle tissue, and in some examples also the action potentials sensed by the sensor S1. The controller 170 may further comprise a communicator, or communication unit 171 as outlined above, which may be configured for receiving and/or transmitting wireless or wired signals to/from outside the body. The communication unit 171 can enable programming the controller 170 form outside of body of the patient such that the operation of the electrode arrangement 150 can be programmed to function optimally.

The controller 170, as well as other implanted components such as the energy source 160 and the charger 190, may be enclosed by an enclosure so as to protect the components from bodily fluids. The enclosures may be an enclosure made from one of or a combination of: a carbon based material (such as graphite, silicon carbide, or a carbon fiber material), a boron material, a polymer material (such as silicone, Peek R, polyurethane, UHWPE or PTFE.), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability, such that migration of fluid through the walls of the enclosure is hindered.

### Communication (Controller; Encryption/Decryption; Authentication/Verification)

The communication between external devices or between an external device and the implant may be encrypted. Any suitable type of encryption may be employed such as symmetric or asymmetric encryption. The encryption may be a single key encryption or a multi-key encryption. In multi-key encryption, several keys are required to decrypt encrypted data. The several keys may be called first key, second key, third key, etc, or first part of a key, second part of the key, third part of the key, etc. The several keys are then combined in any suitable way (depending on the encryption method and use case) to derive a combined key which may be used for decryption. In some cases, deriving a combined key is intended to mean that each key is used one by one to decrypt data, and that the decrypted data is achieved when using the final key.

In other cases, the combination of the several keys results in one “master key” which will decrypt the data. In other words, it is a form of secret sharing, where a secret is divided into parts, giving each participant (external device(s), internal device) its own unique part. To reconstruct the original message (decrypt), a minimum number of parts (keys) is required. In a threshold scheme, this number is less than the total number of parts (e.g. the key at the implant and the key from one of the two external device are needed to decrypt the data). In other embodiments, all keys are needed to reconstruct the original secret, to achieve the combined key which may decrypt the data.

In should be noted that it is not necessary that the generator of a key for decryption is the unit that in the end sends the key to another unit to be used at that unit. In some cases, the generator of a key is merely a facilitator of encryption/decryption, and the working on behalf of another device/user.

A verification unit may comprise any suitable means for verifying or authenticating the use (i.e. user authentication) of a unit comprising or connected to the verification unit, e.g. the external device. For example, a verification unit may comprise or be connected to an interface (UI, GUI) for receiving authentication input from a user. The verification unit may comprise a communication interface for receiving authentication data from a device (separate from the external device) connected to the device comprising the verification unit. Authentication input/data may comprise a code, a key, biometric data based on any suitable techniques such as fingerprint, a palm vein structure, image recognition, face recognition, iris recognition, a retinal scan, a hand geometry, and genome comparison, etc. The verification/authentication may be provided using third-party applications, installed at or in connection with the verification unit.

The verification unit may be used as one part of a two-part authentication procedure. The other part may e.g. comprise conductive communication authentication, sensation authentication, or parameter authentication.

The verification unit may comprise a card reader for reading a smart card. A smart card is a secure microcontroller that is typically used for generating, storing and operating on cryptographic keys. Smart card authentication provides users with smart card devices for the purpose of authentication. Users connect their smart card to the verification unit. Software on the verification unit interacts with the key's material and other secrets stored on the smart card to authenticate the user. In order for the smart card to operate, a user may need to unlock it with a user PIN. Smart cards are considered a very strong form of authentication because cryptographic keys and other secrets stored on the card are very well protected both physically and logically, and are therefore hard to steal.

The verification unit may comprise a personal e-ID that is comparable to, for example, passport and driving license. The e-ID system comprises is a security software installed at the verification unit, and an e-ID which is downloaded from a website of a trusted provided or provided via a smart card from the trusted provider.

The verification unit may comprise software for SMS-based two-factor authentication. Any other two-factor authentication systems may be used. Two-factor authentication requires two things to get authorized: something you know (your password, code, etc.) and something you have (an additional security code from your mobile device (e.g. an SMS, or an e-ID) or a physical token such as a smart card).

Other types of verification/user authentication may be employed. For example, a verification unit which communicates with an external device using visible light instead of wired communication or wireless communication using radio. A light source of the verification unit may transmit (e.g. by flashing in different patterns) secret keys or similar to the external device which uses the received data to verify the user, decrypt data or by any other means perform authentication. Light is easier to block and hide from an eavesdropping adversary than radio waves, which thus provides an advantage in this context. In similar embodiments, electromagnetic radiation is used instead of visible light for transmitting verification data to the external device.

Parameters relating to functionality of the implant may be subject of the communication and comprise sensitive information, for example a status indicator of the implant such as battery level, version of control program, properties of the implant, status of a motor of the implant, etc. Furthermore, data comprising operating instructions may be subject of the communication and comprise other sensitive information, for example a new or updated control program, parameters relating to specific configurations of the implant, etc. Such data may for example comprise instructions on how to operate the electrical stimulation device and/or implantable constriction device, instructions to collect patient data, instructions to transmit feedback, etc. These parameters and data must be protected from being compromised.

### Controller

A controller for controlling the implantable medical device according to any of the embodiments disclosed herein and for communicating with devices external to the body of the patient and/or implantable sensors will now be described in a general way with reference to FIGS. 19A to 19C. FIG. 19A shows a patient when an implantable medical device M comprising a controller 300 has been implanted, such as for example the constriction devices in the form of the exit and entry valves 30, 40 and/or electrical stimulation devices 10 with the controllers C₁ and/or C_{E} described above. The implantable medical device M comprises an active unit 302, which is the part of the electrical stimulation devices and/or mechanical or hydraulic constriction device and which comprises the one or more operable elements, valves, ports, etc. The active unit 302 is directly or indirectly connected to the body of the patient for acting on the intestine. The active unit 302 is connected to the controller 300 via an electrical connection C2. The controller 300 (further described with reference to FIG. 19B) is configured to communicate with an external device 320 (further described with reference to FIG. 19C). The controller 300 can communicate wirelessly with the external device 320 through a wireless connection WL1 and/or through an electrical connection C1.

Referring now to FIG. 19B, one embodiment of the controller 300 will be described in more detail. The controller 300 comprises an internal computing unit 306 configured to control the function performed by the implantable medical device M. The computing unit 306 comprises an internal memory 307 configured to store programs thereon. In the embodiment described in FIG. 19B, the internal memory 307 comprises a first control program 310 which can control the function of the implantable medical device M. The first control program 310 may be seen as a program with minimum functionality to be run at the implantable medical device M only during updating of the second control program 312. When the implantable medical device M is running with the first control program 310, the implantable medical device M may be seen as running in safe mode, with reduced functionality. For example, the first control program 310 may result in that no sensor data is stored in the implantable medical device M while being run, or that no feedback is transmitted from the implantable medical device M while the first control program 310 is running. By having a low-complexity first control program, memory at the implantable medical device M is saved, and the risk of failure of the implantable medical device M during updating of the second control program 312 is reduced.

The second control program 312 is the program controlling the implantable medical device M in normal circumstances, providing the implantable medical device M with full functionality and features.

The memory 307 can further comprise a second, updatable, control program 312. The term updatable is to be interpreted as the program being configured to receive incremental or iterative updates to its code or be replaced by a new version of the code. Updates may provide new and/or improved functionality to the implant as well as fixing previous deficiencies in the code. The computing unit 306 can receive updates to the second control program 312 via the controller 300. The updates can be received wirelessly via WL1 or via the electrical connection C1. As shown in FIG. 19B, the internal memory 307 of the controller 300 can possibly store a third program 314. The third program 314 can control the function of the implantable medical device M, and the computing unit 306 may be configured to update the second program 312 to the third program 314. The third program 314 can be utilized when rebooting an original state of the second program 312. The third program 314 may thus be seen as providing a factory reset of the controller 300, e.g. restore it back to factory settings. The third program 314 may thus be included in the implant 300 in a secure part of the memory 307 to be used for resetting the software (second control program 312) found in the controller 300 to original manufacturer settings.

The controller 300 may comprise a reset function 316 connected to or part of the internal computing unit 306 or transmitted to said internal computing unit 306. The reset function 316 is configured to make the internal computing unit 306 switch from running the second control program 312 to the first control program 310. The reset function 316 may be configured to make the internal computing unit 306 delete the second control program 312 from the memory 307. The reset function 316 can be operated by palpating or pushing/put pressure on the skin of the patient. This may be performed by having a button on the implant. Alternatively, the reset function 316 can be invoked via a timer or a reset module. Temperature sensors and/or pressure sensors can be utilized for sensing the palpating. The reset function 316 may also be operated by penetrating the skin of the patient. It is further plausible that the reset function 316 can be operated by magnetic means. This may be performed by utilizing a magnetic sensor and applying a magnetic force from outside the body. The reset function 316 may be configured such that it responds only to magnetic forces applied for a duration of time exceeding a limit, such as 2 seconds. The time limit may equally plausible be 5 or 10 seconds, or longer. In these cases, the implant may comprise a timer. The reset function 316 may thus include or be connected to a sensor for sensing such magnetic force.

In addition to or as an alternative to the reset function described above, the implant may comprise an internal computing unit 306 (comprising an internal processor) comprising the second control program 312 for controlling a function of the implantable medical device M, and a reset function 318. The reset function 318 may be configured to restart or reset said second control program 312 in response to: i, a timer of the reset function 318 not having been reset, or ii, a malfunction in the first control program 310.

The reset function 318 may comprise a first reset function, such as, for example, a computer operating properly, COP, function connected to the internal computing unit 306. The first reset function may be configured to restart or reset the first or the second control program 312 using a second reset function. The first reset function comprises a timer, and the first or the second control program is configured to periodically reset the timer.

The reset function 318 may further comprise a third reset function connected to the internal computing unit and to the second reset function. The third reset function may in an example be configured to trigger a corrective function for correcting the first 310 or second control program 312, and the second reset function is configured to restart the first 310 or second control program 312 sometime after the corrective function has been triggered. The corrective function may be a soft reset or a hard reset.

The second or third reset function may, for example, configured to invoke a hardware reset by triggering a hardware reset by activating an internal or external pulse generator which is configured to create a reset pulse. Alternatively, the second or third reset function may be implemented by software.

The controller 300 may further comprise an internal wireless transceiver 308. The transceiver 308 communicates wirelessly with the external device 320 through the wireless connection WL1. The transceiver may further communicate with an external device 320, 300 via wireless connection WL2 or WL4. The transceiver may both transmit and receive data via either of the connections C1, WL1. WL2 and WL4. Optionally, the external devices 320 and 300, when present, may communicate with each other, for example via a wireless connection WL3.

The controller 300 can further be electrically connected C1 to the external device 320 and communicate by using the patient's body as a conductor. The controller 300 may thus comprise a wired transceiver 303 or an internal transceiver 303 for the electrical connection C1.

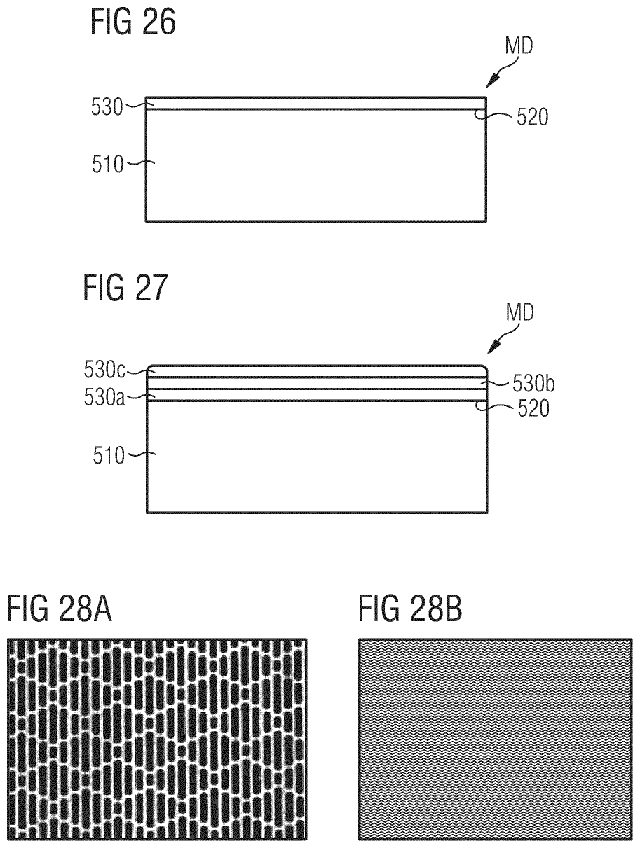

The controller 300 of the implantable medical device M according to FIG. 20B further comprises a feedback unit 349. The feedback unit 349 provides feedback related to the switching from the second control program 312 to the first control program 310. The feedback may for example represent the information on when the update of the software, i.e. the second control program 312, has started, and when the update has finished. This feedback can be visually communicated to the patient, via for example a display on the external device 320. This display may be located on a wristwatch, or a phone, or any other external device 320 coupled to the controller 300. Preferably, the feedback unit 349 provides this feedback signal wirelessly via WL1 to the external device 320. Potentially, the words “Update started”, or “Update finished”, may be displayed to the patient, or similar terms with the same meaning. Another option may be to display different colors, where green for example may mean that the update has finished, and red or yellow that the update is ongoing. Obviously, any color is equally plausible, and the user may choose these depending on personal preference. Another possibility would be to flash a light on the external device 320. In this case the external device 320 comprises the light emitting device(s) needed. Such light may for example be an LED. Different colors may, again, represent the status of the program update. One way of representing that the update is ongoing and not yet finished may be to flash the light, i.e. turning the light on and off. Once the light stops flashing, the patient would be aware of that the update is finished. The feedback may also be audible and provided by the implantable medical device M directly, or by the external device 320. In such cases, the implantable medical device M and external device 320 comprise means for providing audio. The feedback may also be tactile, for example in the form of a vibration that the user can sense. In such a case, either the implantable medical device M or external device 320 comprises means for providing a tactile sensation, such as a vibration and/or a vibrator.

As seen in FIG. 19B, the controller 300 can further comprise a first energy storage unit 40A. The first energy storage unit 40A runs the first control program 310. The controller 300 further comprises a second energy storage unit 40B which runs the second control program 312. This may further increase security during update, since the first control program 310 has its own separate energy storage unit 40A. The first power supply 40A can comprise a first energy storage 304a and/or a first energy receiver 305a. The second energy storage unit 40B can comprise a second energy storage 304b and/or a second energy receiver 305b. The energy can be received wirelessly by inductive or conductive means. An external energy storage unit can for example transfer an amount of wireless energy to the energy receivers 305a. 305b inside the patient's body by utilizing an external coil which induces a voltage in an internal coil (not shown in Figures). It is plausible that the first energy receiver 305a receives energy via a RFID pulse. The feedback unit 349 can provide feedback pertaining to the amount of energy received via the RFID pulse. The amount of RFID pulse energy that is being received can be adjusted based on the feedback, such that the pulse frequency is successively raised until a satisfying level is reached.

The controller 300 of the implantable medical device M according to FIG. 20B further comprises an electrical switch 309. The electrical switch 309 may be mechanically connected to an implantable element configured to exert a force on a body portion of a patient and being configured to be switched as a result of the force exerted on the body portion of a patient exceeding a threshold value. The switch 309 may for example be bonded to a portion of the implantable medical device M in any of the embodiments herein. The switch 309 may alternatively be electrically connected to the implantable medical device M and configured to be switched as a result of the current supplied to the implantable medical device M exceeding a threshold value. The switch 309 may for example be connected to the electrical stimulation devices 10 and/or the constriction devices in the form of the exit and entry valves 30, 40 and configured to be switched if the current to the implantable medical device M exceeds a threshold value. Such a switch may for example be a switch 309 configured to switch if exposed to a temperature exceeding a threshold value, such as a bimetal switch which is switched by the heat created by the flow of current to e.g. the electrodes of the electrical stimulation devices 10 or a motor of the mechanical or hydraulic constriction devices. In the alternative, the switch 309 configured to switch if exposed to a temperature exceeding a threshold value may be placed at a different location on the implantable medical device M to switch in case of exceeding temperatures, thereby hindering the implantable medical device M from overheating which may cause tissue damage.

The switch 309 may either be configured to cut the power to the operation device or to generate a control signal to the processor 306 of the implantable controller 300, such that the controller 300 can take appropriate action, such as reducing power or turning off the operation of the implantable medical device M.

The external device 320 is represented in FIG. 19C. The external device 320 can be placed anywhere on the patient's body, preferably at a convenient and comfortable place. The external device 320 may be a wristband, and/or have the shape of a wristwatch. It is also plausible that the external device is a mobile phone or other device not attached directly to the patient. The external device 320 as shown in FIG. 19C comprises a wired transceiver 323, and an energy storage 324. It also comprises a wireless transceiver 328 and an energy transmitter 325. It further comprises a computing unit 326 and a memory 327. The feedback unit 322 in the external device 320 is configured to provide feedback related to the computing unit 326. The feedback provided by the feedback unit 322 may be visual. The external device 320 may have a display showing such visual feedback to the patient. It is equally plausible that the feedback is audible and that the external device 320 comprises means for providing audio. The feedback given by the feedback unit 322 may also be tactile, such as vibrating. The feedback may also be provided in the form of a wireless signal WL1, WL2, WL3, WL4.

The second, third or fourth communication method WL2, WL3, WL4 may be a wireless form of communication. The second, third or fourth communication method WL2, WL3, WL4 may preferably be a form of electromagnetic or radio-based communication. The second, third and fourth communication method WL2, WL3, WL4 may be based on telecommunication methods. The second, third or fourth communication method WL2, WL3, WL4 may comprise or be related to the items of the following list: Wireless Local Area Network (WLAN), Bluetooth, Bluetooth 5, BLE, GSM or 2G (2nd generation cellular technology), 3G, 4G or 5G.

The external device 320 may be adapted to be in electrical connection C1 with the implantable medical device M, using the body as a conductor. The electrical connection C1 is in this case used for conductive communication between the external device 320 and the implantable medical device M.

### Encryption/Decryption

In one embodiment, the communication between controller 300 and the external device 320 over either of the communication methods WL2, WL3, WL4, C1 may be encrypted and/or decrypted with public and/or private keys, now described with reference to FIGS. 19A to 19C. For example, the controller 300 may comprise a private key and a corresponding public key, and the external device 320 may comprise a private and a corresponding public key.

The controller 320 and the external device 320 may exchange public keys and the communication may thus be performed using public key encryption. The person skilled in the art may utilize any known method for exchanging the keys.

The controller may encrypt data to be sent to the external device 320 using a public key corresponding to the external device 320. The encrypted data may be transmitted over a wired, wireless or electrical communication channel C1, WL1, WL2, WL3 to the external device. The external device 320 may receive the encrypted data and decode it using the private key comprised in the external device 320, the private key corresponding to the public key with which the data has been encrypted. The external device 320 may transmit encrypted data to the controller 300. The external device 320 may encrypt the data to be sent using a public key corresponding to the private key of the controller 300. The external device 320 may transmit the encrypted data over a wired, wireless or electrical connection C1, WL1, WL2, WL3, WL4, directly or indirectly, to the controller of the implant. The controller may receive the data and decode it using the private key comprised in the controller 300.

In an alternative to the public key encryption, described with reference to FIGS. 19A to 19C, the data to be sent between the controller 300 of the implantable medical device M and an external device 320, 330 or between an external device 320, 330 and the controller 300 may be signed. In a method for sending data from the controller 300 to the external device 320, 330, the data to be sent from the controller 300 may be signed using the private key of the controller 300. The data may be transmitted over a communication channel or connection C1, WL1, WL2, WL3, WL4. The external device 320, 330 may receive the message and verify the authenticity of the data using the public key corresponding to the private key of the controller 300. In this way, the external device 320, 330 may determine that the data were sent from the controller 300 and not from another device or source.

A method for communication between an external device 320 and the controller 300 of the implantable medical device M using a combined key is now described with reference to FIGS. 19A to 19C. A first step of the method comprises receiving, at the implant, by a wireless transmission WL1. WL2, WL3, WL4 or otherwise, a first key from an external device 320, 330. The method further comprises receiving, at the implant, by a wireless transmission WL1, WL2, WL3, a second key. The second key may be generated by a second external device, separate from the external device 320, 330 or by another external device being a generator of the second key on behalf of the second external device 320, 330. The second key may be received at the implant from any one of: the external device 320, the second external device 330, and the generator of the second key. The second external device may be controlled by a caretaker or any other stakeholder. Said another external device may be controlled by a manufacturer of the implant, or medical staff, caretaker, etc.

In case the controller 300 is receiving the second key from the external device 320, this means that the second key is routed through the external device from the second external device 330 or from another external device (generator). The routing may be performed as described herein under the tenth aspect. In these cases, the implant and/or external device(s) comprises the necessary features and functionality (described in the respective sections of this document) for performing such routing. Using the external device 320 as a relay, with or without verification from the patient, may provide an extra layer of security as the external device 320 may not need to store or otherwise handle decrypted information. As such, the external device 320 may be lost without losing decrypted information. The controller 300 comprises a computing unit 306 configured for deriving a combined key by combining the first key and the second key with a third key held by the controller 300, for example in memory 307 of the controller 300. The third key may for example be a license number of the implant or a chip number of the implantable medical device M. The combined key may be used for decrypting, by the computing unit 306, encrypted data transmitted by a wireless transmission WL1 from the external device 320 to the controller 300. Optionally, the decrypted data may be used for altering, by the computing unit 306, an operation of the implantable medical device M. The altering of an operation of the implantable medical device M may comprise controlling or switching an active unit 302 of the implant. In some embodiments, the method further comprises at least one of the steps of, based on the decrypted data, updating a control program running in the controller 300, and operating the implantable medical device M using operation instructions in the decrypted data.

Methods for encrypted communication between an external device 320 and the controller 300 may comprise:
receiving, at the external device 320, by a wireless transceiver 328, a first key, the first key being generated by a second external device 330, separate from the external device 320 or by another external device being a generator of the second key on behalf of the second external device 330, the first key being received from any one of the second external device 330 and the generator of the second key,
receiving, at the external device 320 by the wireless transceiver 328, a second key from the controller 300,
deriving a combined key, by a computing unit 326 of the external device 320, by combining the first key and the second key with a third key held by the external device 320 (e.g. in memory 307),
transmitting encrypted data from the implant to the external device and receiving the encrypted data at the external device by the wireless transceiver 328, and
decrypting, by the computing unit 326, the encrypted data, in the external device 320, using the combined key.

As described above, further keys may be necessary to decrypt the data. Consequently, the wireless transceiver 328 is configured for:
receiving a fourth key from a third external device,

wherein the computing unit 326 is configured for:
deriving a combined key by combining the first, second and fourth key with the third key held by the external device, and
1 decrypting the encrypted data using the combined key.

These embodiments further increase the security in the communication. The computing unit 326 may be configured to confirm the communication between the implant and the external device, wherein the confirmation comprises:
measuring a parameter of the patient, by the external device 320, receiving a measured parameter of the patient, from the implantable medical device M,
comparing the parameter measured by the implantable medical device M with the parameter measured by the external device 320,
performing confirmation of the connection based on the comparison, and
as a result of the confirmation, decrypting the encrypted data, in the external device, using the combined key.

The keys described in this section may in some embodiments be generated based on data sensed by sensors described hereinafter, e.g. using the sensed data as seed for the generated keys. A seed is an initial value that is fed into a pseudo random number generator to start the process of random number generation. The seed may thus be made hard to predict without access to or knowledge of the physiological parameters of the patient which it is based on, providing an extra level of security to the generated keys.

### Method of Communication

A method of communication between an external device 320 and an implantable medical device M is now described with reference to FIGS. 19A to 19C, when the implantable medical device M is implanted in a patient and the external device 320 is positioned external to the body of the patient. The external device 320 is adapted to be in electrical connection C1 with the controller 300, using the body as a conductor. The electrical connection C1 is used for conductive communication between the external device 320 and the implantable medical device M. The implantable medical device M comprises the controller 300. Both the controller 300 and the external device 320 comprise a wireless transceiver 308 for wireless communication C1 between the controller 300 and the external device 320. The wireless transceiver 308 (included in the controller 300) may in some embodiments comprise sub-transceivers for receiving data from the external device 320 and other external devices, e.g. using different frequency bands, modulation schemes, etc.

In a first step of the method, the electrical connection C1 between the controller 300 and the external device 320 is confirmed and thus authenticated. The confirmation and authentication of the electrical connection may be performed as described hereinafter. In these cases, the implant and/or external device(s) comprise the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

The controller 300 of the implanted medical device M may comprise a first transceiver 303 configured to be in electrical connection C1 with the external device 320, using the body as a conductor. Alternatively, the first transceiver 303 of the controller 300 may be wireless. The external device 320 may comprise a first external transmitter 323 configured to be in electrical connection C1 with the implanted medical device M, using the body as a conductor, and a wireless transmitter configured to transmit wireless communication WL1 to the controller 300. Alternatively, the first external transmitter 323 of the external device 320 may be wireless. The first external transmitter 323 and the wireless transmitter of the external device 320 may be the same or separate transmitters.

The controller 300 may comprise a computing unit 306 configured to confirm the electrical connection between the external device 320 and the internal transceiver 303 and accept wireless communication WL1 (of the data) from the external device 320 on the basis of the confirmation.

Data is transmitted from the external device 320 to the controller 300 wirelessly, e.g. using the respective wireless transceivers of the controller 300 and the external device 320. Data may alternatively be transmitted through the electrical connection C1. As a result of the confirmation, the received data may be used for instructing the implantable medical device M. For example, a control program 310 running in the controller 300 may be updated or the controller 300 may be operated using operation instructions in the received data. This may be handled by the computing unit 306.

The method may comprise transmitting data from the external device 320 to the controller 300 wirelessly which may comprise transmitting encrypted data wirelessly. To decrypt the encrypted data (for example using the computing unit 306), several methods may be used.

In one embodiment, a key is transmitted using the confirmed conductive communication channel C1 (i.e. the electrical connection) from the external device 320 to the controller 300. The key is received at the controller (by the first internal transceiver 303). The key is then used for decrypting the encrypted data.

In some embodiments the key is enough to decrypt the encrypted data. In other embodiments, further keys are necessary to decrypt the data. In one embodiment, a key is transmitted using the confirmed conductive communication channel C1 (i.e. the electrical connection) from the external device 320 to the controller 300. The key is received at the controller 300 (by the first internal transceiver 303). A second key is transmitted (by the wireless transceiver 208) from the external device 320 using the wireless communication WL1 and received at the controller 300 by the wireless transceiver 308. The computing unit 306 then derives a combined key from the key and second key and uses this for decrypting the encrypted data.

In yet other embodiments, a key is transmitted using the confirmed conductive communication channel C1 (i.e. the electrical connection) from the external device 320 to the controller 300. The key is received at the controller (by the first internal transceiver 303). A third key is transmitted from a second external device 330, separate from the external device 320, to the implant wirelessly via WL2. The third key may be received by a second wireless receiver (part of the wireless transceiver 308) of the controller 300 configured for receiving wireless communication via WL2 from the second external device 330.

The first and third key may be used to derive a combined key by the computing unit 306, which then decrypts the encrypted data. The decrypted data is then used for instructing the implantable medical device M as described above.

The second external device 330 may be controlled by, for example, a care person to further increase security and validity of data sent and decrypted by the controller 300.

It should be noted that in some embodiments, the external device is further configured to receive WL2 secondary wireless communication from the second external device 330, and transmit data received from the secondary wireless communication WL2 to the implantable medical device M. This routing of data may be achieved using the wireless transceivers 308, 208 (i.e. the wireless connection WL1, or by using a further wireless connection WL4 between the controller 300 and the external device 320. In these cases, the implant and/or external device(s) comprise(s) the necessary features and functionality for performing such routing. Consequently, in some embodiments, the third key is generated by the second external device 330 and transmitted via WL2 to the external device 320 which routes the third key to the controller 300 to be used for decryption of the encrypted data. In other words, the step of transmitting a third key from a second external device, separate from the external device, to the implant wirelessly, comprises routing the third key through the external device 320. Using the external device 320 as a relay, with or without verification by the patient, may provide an extra layer of security as the external device 320 may not need to store or otherwise handle decrypted information. As such, the external device 320 may be lost without losing decrypted information.

In yet other embodiments, a key is transmitted using the confirmed conductive communication channel C1 (i.e. the electrical connection) from the external device 320 to the controller 300. The key is received at the implant (by the first internal transceiver 303). A second key is transmitted from the external device 320 to the controller 300 wirelessly via WL1, received at the controller 300. A third key is transmitted from the second external device, separate from the external device 320, to the controller 300 wirelessly via WL4. Encrypted data transmitted from the external device 320 to the controller 300 is then decrypted using a derived combined key from the key, the second key and the third key. The external device may be a wearable external device.

The external device 320 may be a handset. The second external device 330 may be a handset or a server or may be cloud-based.

In some embodiments, the electrical connection C1 between the external device 320 and the controller 300 is achieved by placing a conductive member 321, configured to be in connection with the external device 320, in electrical connection with a skin of the patient for conductive communication C1 with the implant. In these cases, the implant and/or external device(s) comprise(s) the necessary features and functionality (described in the respective sections of this document) for performing such conductive communication. The communication may thus be provided with an extra layer of security in addition to the encryption by being electrically confined to the conducting path e.g. external device 320, conductive member 321, conductive connection C1, controller 300, meaning the communication will be excessively difficult to be intercepted by a third party not in physical contact with, or at least proximal to, the patient.

### Authentication/Verification

To further increase security of the communication between the controller 300 and the external device 320, different types of authentication, verification and/or encryption may be employed. In some embodiments, the external device 320 comprises a verification unit 340. The verification unit 340 may be any type of unit suitable for verification of a user, i.e. configured to receive authentication input from a user, for authenticating the conductive communication between the implant and the external device. In some embodiments, the verification unit and the external device comprises means for collecting authentication input from the user (which may or may not be the patient). Such means may comprise a fingerprint reader, a retina scanner, a camera, a GUI for inputting a code, a microphone, a device configured to draw blood, etc. The authentication input may thus comprise a code or anything based on a biometric technique selected from the list of: a fingerprint, a palm vein structure, image recognition, face recognition, iris recognition, a retinal scan, a hand geometry, and genome comparison. The means for collecting the authentication input may alternatively be part of the conductive member 321 which comprise any of the above examples of functionality, such as a fingerprint reader or other type of biometric reader.

In some embodiments, the security may thus be increased by receiving an authentication input from a user by the verification unit 340 of the external device 320 and authenticating the conductive communication between the controller 300 and the external device using the authentication input. Upon a positive authentication, the conductive communication channel C1 may be employed for comprising transmitting a conductive communication to the controller 300 by the external device 320 and/or transmitting a conductive communication to the external device 320 by the controller 300. In other embodiments, a positive authentication is needed prior to operating the implantable medical device M based on received conductive communication and/or updating a control program running in the controller 300 as described above.

FIGS. 19A to 19C further show that the implantable medical device M is connected to a sensation generator 381. The sensation generator 381 may be configured to generate a sensation. The sensation generator 381 may be contained within the implantable medical device M or be a separate unit. The sensation generator 381 may be implanted. The sensation generator 381 may also be located so that it is not implanted as such but still is in connection with a patient so that only the patient may experience sensations generated. The controller 300 is configured for storing authentication data, related to the sensation generated by the sensation generator 381.

The controller 300 is further configured for receiving input authentication data from the external device 320. Authentication data related to the sensation generated may be stored by a memory 307 of the controller 300. The authentication data may include information about the generated sensation such that it may be analyzed, e.g. compared, to input authentication data to authenticate the connection, communication or device. Input authentication data relates to information generated by a patient input to the external device 320. The input authentication data may be the actual patient input or an encoded version of the patient input, encoded by the external device 320. Authentication data and input authentication data may comprise a number of sensations or sensation components.

The authentication data may comprise a timestamp. The input authentication data may comprise a timestamp of the input from the patient. The timestamps may be a time of the event such as the generation of a sensation by the sensation generator 381 or the creation of input authentication data by the patient. The timestamps may be encoded. The timestamps may feature arbitrary time units, i.e. not the actual time. Timestamps may be provided by an internal clock 360 of the controller 300 and an external clock 362 of the external device 320. The clocks 360, 362 may be synchronized with each other. The clocks 360, 362 may be synchronized by using a conductive connection C1 or a wireless connection WL1 for communicating synchronization data from the external device 320, and its respective clock 362, to the controller 300, and its respective clock 360, and vice versa. Synchronization of the clocks 360, 362 may be performed continuously and may not be reliant on secure communication.

Authentication of the connection may comprise calculating a time difference between the timestamp of the sensation and the timestamp of the input from the patient, and upon determining that the time difference is less than a threshold, authenticating the connection. An example of a threshold may be 1 s. The analysis may also comprise a low threshold as to filter away input from the patient that is faster than normal human response times. The low threshold may e.g. be 50 ms.

Authentication data may comprise a number of times that the sensation is generated by the sensation generator, and wherein the input authentication data comprises an input from the patient relating to a number of times the patient detected the sensation. Authenticating the connection may then comprise: upon determining that the number of times that the authentication data and the input authentication data are equal, authenticating the connection.

A method of authenticating the connection between the implantable medical device M and the external device 320 accordingly includes the following steps.

Generating, by the sensation generator 381, a sensation detectable by a sense of the patient. The sensation may comprise a plurality of sensation components. The sensation or sensation components may comprise a vibration (e.g. a fixed frequency mechanical vibration), a sound (e.g. a superposition of fixed-frequency mechanical vibrations), a photonic signal (e.g. a non-visible light pulse such as an infrared pulse), a light signal (e.g. a visual light pulse), an electrical signal (e.g. an electrical current pulse) or a heat signal (e.g. a thermal pulse). The sensation generator may be implanted, configured to be worn in contact with the skin of the patient or capable of creating sensation without being in physical contact with the patient, such as a beeping alarm. Sensations may be configured to be consistently felt by a sense of the patient while not risking harm to or affecting internal biological processes of the patient.

Storing, by the controller 300, authentication data related to the generated sensation.

Providing, by the patient, input to the external device, resulting in input authentication data. Providing the input may e.g. comprise engaging an electrical switch, using a biometric input sensor or entering the input into a digital interface running on the external device 320, to name just a few examples.

Transmitting the input authentication data from the external device to the controller 300. If the step was performed, the analysis may be performed by the controller 300.

Transmitting the authentication data from the implantable medical device M to the external device 320. If the step was performed, the analysis may be performed by the external device 320. The wireless connection WL1 or the conductive connection C1 may be used to transmit the authentication data or the input authentication data.

Authenticating the connection based on an analysis of the input authentication data and the authentication data e.g. by comparing a number of sensations generated and experienced or comparing timestamps of the authentication data and the input authentication data. If the step was performed, the analysis may be performed by the implantable medical device M.

Communicating further data between the controller 300 and the external device 320 following positive authentication. The wireless connection WL1 or the conductive connection C1 may be used to communicate the further data. The further data may comprise data for updating a control program 310 running in the controller 300 or operation instructions for operating the implantable medical device M.

If the analysis was performed by the controller 300, the external device 320 may continuously request or receive information of an authentication status of the connection between the controller 300 and the external device 320, and upon determining, at the external device 320, that the connection is authenticated, transmitting further data from the external device 320 to the controller 300.

If the analysis was performed by the external device 320, the controller 300 may continuously request or receive information of an authentication status of the connection between the controller 300 and the external device 320, and upon determining, at the controller 300, that the connection is authenticated, transmitting further data from the controller 300 to the external device 320.

A main advantage of authenticating a connection according to this method is that only the patient may be able to experience the sensation. Thus, only the patient may be able to authenticate the connection by providing authentication input corresponding to the sensation generation.

### Security Module

According to one embodiment described with reference to FIG. 19A-19C, the communication unit 300 or internal controller 300 or control unit 300 comprises a wireless transceiver 308 for communicating wirelessly with an external device, a security module 389, and a central unit, also referred to herein as a computing unit 306 306, which is to be considered as equivalent. The central unit 306 is configured to be in communication with the wireless transceiver 308, the security module 389 and the implantable medical device or active unit 302. The wireless transceiver 308 is configured to receive communication from the external device 320 including at least one instruction to the implantable medical device MD and transmit the received communication to the central unit or computing unit 306. The central unit or computing unit 306 is configured to send secure communication to the security module 389, derived from the received communication from the external device 320, and the security module 389 is configured to decrypt at least a portion of the secure communication and verify the authenticity of the secure communication. The security module is further configured to transmit a response communication to the central unit or computing unit 306 and the central unit or computing unit is configured to communicate the at least one instruction to the active unit 302. In the embodiment shown in FIG. 19A-19C, the at least one instruction is based on the response communication, or a combination of the response communication and the received communication from the external device 320.

In the embodiment shown in FIG. 19A-19C, the security module 389 comprises a set of rules for accepting communication from the central unit or computing unit 306. In the embodiment shown in FIG. 19A-19C, the wireless transceiver 308 is configured to be able to be placed in an off-mode, in which no wireless communication can be transmitted or received by the wireless transceiver 308. The set of rules comprises a rule stipulating that communication from the central unit or computing unit 306 to the security module 389 or to the active unit 302 is only accepted when the wireless transceiver 308 is placed in the off-mode.

In the embodiment shown in FIG. 19A-19C, the set of rules comprises a rule stipulating that communication from the central unit or computing unit 306 is only accepted when the wireless transceiver 308 has been placed in the off-mode for a specific time period.

In the embodiment shown in FIG. 19A-19C, the central unit or computing unit 306 is configured to verify a digital signature of the received communication from the external device 320. The digital signature could be a hash-based digital signature which could be based on a biometric signature from the patient or a medical professional. The set of rules further comprises a rule stipulating that communication from the central unit 306 is only accepted when the digital signature of the received communication has been verified by the central unit 306. The verification could for example comprise the step of comparing the digital signature or a portion of the digital signature with a previously verified digital signature stored in the central unit 306. The central unit 306 may be configured to verify the size of the received communication from the external device and the set of rules could comprise a rule stipulating that communication from the central unit 306 is only accepted when the size of the received communication has been verified by the central unit 306. The central unit could thus have a rule stipulating that communication above or below a specified size range is to be rejected.

In the embodiment shown in FIG. 19A-19C, the wireless transceiver is configured to receive a message from the external device 320 being encrypted with at least a first and second layer of encryption. The central unit 306 the decrypts the first layer of decryption and transmit at least a portion of the message comprising the second layer of encryption to the security model 389. The security module 389 then decrypts the second layer of encryption and transmits a response communication to the central unit 306 based on the portion of the message decrypted by the security module 389.

In the embodiment shown in FIG. 19A-19C, the central unit 306 is configured to decrypt a portion of the message comprising a digital signature, such that the digital signature can be verified by the central unit 306, also the central unit 306 is configured to decrypt a portion of the message comprising message size information, such that the message size can be verified by the central unit 306.

In the embodiment shown in FIG. 19A-19C, the central unit 306 is configured to decrypt a first and second portion of the message, and the first portion comprises a checksum for verifying the authenticity of the second portion.

In the embodiment shown in FIG. 19A-19C, the response communication transmitted from the security module 389 comprises a checksum, and the central unit 306 is configured to verify the authenticity of at least a portion of the message decrypted by the central unit 306 using the received checksum, i.e. by adding portions of the message decrypted by the central unit 306 and comparing the sum to the checksum.

In the embodiment shown in FIG. 19A-19C, the set of rules further comprise a rule related to the rate of data transfer between the central unit 306 and the security module 389. The rule could stipulate that the communication should be rejected or aborted if the rate of data transfer exceeds a set maximum rate of data transfer, which may make it harder for unauthorized persons to inject malicious code or instructions to the medical implant.

In the embodiment shown in FIG. 19A-19C, the security module 389 is configured to decrypt a portion of the message comprising the digital signature being encrypted with the second layer of encryption, such that the digital signature can be verified by the security module 389. The security module 389 then transmits a response communication to the central unit 306 based on the outcome of the verification, which can be used by the central unit 306 for further decryption of the message or for determining if instructions in the message should be communicated to the active unit 302.

In the embodiment shown in FIG. 19A-19C, the central unit 306 is only capable of decrypting a portion of the received communication from the external device 320 when the wireless transceiver 308 is placed in the off-mode. In the alternative, or as an additional layer of security, the central unit 306 may be limited such that the central unit 306 is only capable of communicating instructions to the active unit 302 of the implantable medical device MD when the wireless transceiver 308 is placed in the off-mode. This ensures that no attacks can take place while the central unit 306 is communicating with the active unit 302.

In the embodiment shown in FIG. 19A-19C, the implantable controller 300 is configured to receive, using the wireless transceiver 308, a message from the external device 320 comprising a first non-encrypted portion and a second encrypted portion. The implantable controller 300 (e.g. the central unit 306 or the security module 389) then decrypts the encrypted portion, and uses the decrypted portion to verify the authenticity of the non-encrypted portion. As such, computing power and thereby energy can be saved by not encrypting the entire communication, but rather only the portion required to authenticate the rest of the message (such as a checksum and/or a digital signature)

In the embodiment shown in FIG. 19A-19C, the central unit 306 is configured to transmit an encrypted portion to the security module 389 and receive a response communication from the security module 389 based on information contained in the encrypted portion being decrypted by the security module. The central unit 306 is then configured to use the response communication to verify the authenticity of the non-encrypted portion. The non-encrypted portion could comprise at least a portion of the at least one instruction to the implantable medical device 306.

In the embodiment shown in FIG. 19A-19C, the implantable controller 300 is configured to receive, using the wireless transceiver 308, a message from the external device 320 comprising information related to at least one of: a physiological parameter of the patient and a physical parameter of the implanted medical device MD, and use the received information to verify the authenticity of the message. The physiological parameter of the patient could be a parameter such as a parameter based on one or more of: a temperature, a heart rate and a saturation value.

The physical parameter of the implanted medical device MD could comprise at least one of a current setting or value of the implanted medical device MD, a prior instruction sent to the implanted medical device MD or an ID of the implanted medical device MD.

The portion of the message comprising the information related to the physiological parameter of the patient and/or physical or functional parameter of the implanted medical device MD could be encrypted, and the central unit 306 may be configured to transmit the encrypted portion to the security module 389 and receive a response communication from the security module 389 based on the information having been decrypted by the security module 389.

In the embodiment shown in FIG. 19A-19C, the security module 389 is a hardware security module comprising at least one hardware-based key. The security module 389 may have features that provide tamper evidence such as visible signs of tampering or logging and alerting. It may also be so that the security module 389 is “tamper resistant”, which makes the security module 389 inoperable in the event that tampering is detected. For example, the response to tampering could include deleting keys is tampering is detected. The security module 389 could comprise one or more secure cryptoprocessor chip. The hardware-based key(s) in the security module 389 could have a corresponding hardware-based key placeable in the external device 320. The corresponding external hardware-based key could be placed on a key-card connectable to the external device 320.

In alternative embodiments, the security module 389 is a software security module comprising at least one software-based key, or a combination of a hardware and software-based security module and key. The software-based key may correspond to a software-based key in the external device 320. The software-based key may correspond to a software-based key on a key-card connectable to the external device 320.

In the embodiment shown in FIG. 19A-19C, the external device 320 is a handheld external device, however, in alternative embodiments, the external device may be a remote external device or a cloud based external device

In the embodiment shown in FIG. 19A-19C, the at least one instruction to the implantable medical device MD comprises an instruction for changing an operational state of the implantable medical device MD.

In the embodiment shown in FIG. 19A-19C, the wireless transceiver 308 is configured to communicate wirelessly with the external 320 device using electromagnetic waves at a frequency below 100 kHz, or more specifically below 40 kHz. The wireless transceiver 308 is thus configured to communicate with the external device 320 using “Very Low Frequency” communication (VLF). VLF signals have the ability to penetrate a titanium housing of the implantable medical device MD, such that the electronics of the implantable medical device MD can be completely encapsulated in a titanium housing.

The wireless transceiver 308 is configured to communicate wirelessly with the external device 320 using a first communication protocol and the central unit 306 is configured to communicate with the security module 389 using a second, different, communication protocol. This adds an additional layer of security as security structures could be built into the electronics and/or software in the central unit 306 enabling the transfer from a first to a second communication protocol. The wireless transceiver 308 may be configured to communicate wirelessly with the external device using a standard network protocol, which could be one of an RFIDtype protocol, a WLAN-type protocol, a Bluetooth-(BT)-type protocol, a BLE-type protocol, an NFC-type protocol, a 3G/4G/5G-type protocol, and a-GSM type protocol. In the alternative, or as a combination, the wireless transceiver 308 could be configured to communicate wirelessly with the external device 320 using a proprietary network protocol. The wireless transceiver 308 could comprises a Ultra-Wide Band (UWB) transceiver and the wireless communication between the implantable controller 300 and the external device 320 could thus be based on UWB. The use of UWB technology enables positioning of the remote control 320″ which can be used by the implanted medical device MD as a way to establish that the external device 320 is at a position which the implanted medical device MD and/or the patient can acknowledge as being correct, e.g. in the direct proximity to the medical device MD and/or the patient, such as within reach of the patient and/or within 1 or 2 meters of the implanted medical device MD. In the alternative, a combination of UWB and BT could be used, in which case the UWB communication can be used to authenticate the BT communication, as it is easier to transfer large data sets using BT.

### Variable Impedance

According to one embodiment described with reference to FIG. 19A-19C, the communication unit 300 or controller of the implantable medical device MD comprises a receiving unit 305 or energy receiver 305 comprising a coil 192 (specifically shown in FIG. 19B) configured for receiving transcutaneously transferred energy. The receiving unit further comprises a measurement unit 194 configured to measure a parameter related to the energy received by the coil 192 and a variable impedance 193 electrically connected to the coil 192. The receiving unit 305 further comprises a switch 195a placed between the variable impedance 193 and the coil 192 for switching off the electrical connection between the variable impedance 193 and the coil 192. The communication unit 300 or controller 300 is configured to control the variable impedance 193 for varying the impedance and thereby tune the coil 192 based on the measured parameter. The communication unit 300 or controller 300 is further configured to control the switch 195a for switching off the electrical connection between the variable impedance 193 and the coil 192 in response to the measured parameter exceeding a threshold value. The controller 300 may further be configured to vary the variable impedance in response to the measured parameter exceeding a threshold value. As such, the coil can be tuned or turned off to reduce the amount of received energy if the amount of received energy becomes excessive. The measurement unit 194 is configured to measure a parameter related to the energy received by the coil 192 over a time period and/or measure a parameter related to a change in energy received by the coil 192 by for example measure the derivative of the received energy over time. The variable impedance 193 is in the embodiment shown in FIG. 19B′ placed in series with the coil 192. In alternative embodiments it is however conceivable that the variable impedance is placed parallel to the coil 192.

The first switch 195a is placed at a first end portion 192a of the coil 192, and the implantable medical device MD further comprises a second switch 195b placed at a second end portion of the coil 192, such that the coil 192 can be completely disconnected from other portions of the implantable medical device MD. The receiving unit 305 is configured to receive transcutaneously transferred energy in pulses according to a pulse pattern. The measurement unit 194 is in the embodiment shown in FIG. 19B′ configured to measure a parameter related to the pulse pattern. The controller 300 is configured to control the variable impedance in response to the pulse pattern deviating from a predefined pulse pattern. The controller 300 is configured to control the switch 195a for switching off the electrical connection between the variable impedance 193 and the coil 192 in response to the pulse pattern deviating from a predefined pulse pattern. The measurement unit is configured to measure a temperature in the implantable medical device MD or in the body of the patient, and the controller 300 is configured to control the first and second switch 195a. 195b in response to the measured temperature.

The variable impedance 193 may comprise a resistor and a capacitor and/or a resistor and an inductor and/or an inductor and a capacitor. The variable impedance 193 may comprise a digitally tuned capacitor or a digital potentiometer. The variable impedance 193 may comprise a variable inductor. The first and second switch comprises a semiconductor, such as a MOSFET. The variation of the impedance is configured to lower the active power that is received by the receiving unit. As can be seen in FIG. 19B′, the variable impedance 193, the first and second switch 195a. 195b and the measurement unit 194 are connected to the communication unit/controller 300 and the receiving unit 305 is connected to an energy storage unit 40 such that the energy storage unit 40 can store energy received by the receiving unit 305.

Plurality of External Devices with Different Levels of Authority for Increased Security

FIG. 20 shows one embodiment of a system for charging, programming and communicating with the controller 300 of the implanted medical device MD. FIG. 20 further describes the communication and interaction between different external devices which may be devices held and operated by the patient, by a health care provider (HCP) or by a Dedicated Data Infrastructure (DDI), which is an infrastructure supplier for example by the manufacturer of the implanted medical device MD or the external devices 320′. 320″. 320″ “. The system of the embodiment of FIG. 20 comprises three external devices 320′. 320”. 320″” capable of communicating with the controller 300. The basic idea is to ensure the security of the communication with, and the operation of, the medical device MD by having three external devices 320′. 320″. 320″” with different levels of authority. The lowest level of authority is given to the patient-operated remote control 320″. The remote control 320″ is authorized to operate functions of the implanted medical device MD via the implanted controller 300, on the basis of patient input. The remote control 320″ is further authorized to fetch some necessary data from the controller 300. The remote control 320″ is capable of operating the controller 300 only by communicating with the software currently running on the controller 300 with the current settings or the software. The next level of authority is given to the Patient External Interrogation Device (P-EID) 320″” which is a charging and communication unit which is held by the patient but is partially remotely operated by the Health Care Provider (HCP). (This is usually a medical doctor of the clinic providing the treatment with help of the implanted medical device MD). The P-EID 320″” is authorized to make setting changes to the software running on the controller 300 of the implanted medical device MD when remotely operated by the HCP. The highest level of authority is given to the HCP-EID 320′. The HCP-EID 320′ is a charging and communication unit which is held by the HCP physically at the clinic of the HCP. The HCP-EID 320′ is authorized to freely alter or replace the software running on the controller 300 when the patient is physically in the clinic or the HCP.

Starting from the lowest level of authority, the remote control 320″ comprises a wireless transceiver 328 for communicating with the implanted medical device MD. The remote control 320″ is capable of controlling the operation of the implanted medical device MD via the controller 300, by controlling pre-set functions of the implantable medical device MD, e.g. for operating an active portion of the implanted medical device MD for performing the intended function of the implanted medical device MD. In the embodiment shown in FIG. 20, the wireless transceiver 328 comprises a Bluetooth (BT) transceiver, and the remote control 320″ is configured to communicate with implanted medical device MD using BT. In an alternative configuration, the remote control 320″ communicates with the implanted medical device MD using a combination of Ultra-Wide Band (UWB) wireless communication and BT. The use of UWB technology enables positioning of the remote control 320″ which can be used by the implanted medical device MD as a way to establish that the remote control 320″ is at a position which the implanted medical device MD and/or the patient can acknowledge as being correct, e.g. in the direct proximity to the medical device MD and/or the patient, such as within reach of the patient and/or within 1 or 2 meters of the implanted medical device MD.

UWB communication is performed by the generation of radio energy at specific time intervals and occupying a large bandwidth, thus enabling pulse-position or time modulation. The information can also be modulated on UWB signals (pulses) by encoding the polarity of the pulse and/or its amplitude and/or by using orthogonal pulses. A UWB radio system can be used to determine the “time of flight” of the transmission at various frequencies. This helps to overcome multipath propagation since some of the frequencies have a line-of-sight trajectory while other indirect paths have longer delay. With a cooperative symmetric two-way metering technique, distances can be measured with high resolution and accuracy. UWB is useful for real-time location systems, and its precision capabilities and low power make it well-suited for radio frequency-sensitive environments.

In embodiments in which a combination of BT and UWB technology is used, the UWB technology may be used for location-based authentication of the remote control 320″, whereas the communication and/or data transfer can take place using BT. The UWB signal can in some embodiments also be used as a wake-up signal for the controller 300, or for the BT transceiver such that the BT transceiver in the implanted medical device MD can be turned off when not in use, which eliminates the risk that the BT is intercepted, or that the controller 300 of the implanted medical device MD is hacked by means of BT communication. In embodiments in which a BT/UWB combination is used, the UWB connection may be used also for the transmission of data. In the alternative, the UWB connection can be used for the transmission of some portions of the data, such as sensitive portions of the data, or for the transmission of keys for the unlocking of encrypted communication sent over BT.

The remote control 320″ comprises control logic which runs a control logic application for communicating with the implanted medical device MD. The control logic can receive input directly from control buttons 335 arranged on the remote control 320″ or from a control interface 334i displayed on a display device 334 operated by the patient. In the embodiments in which the remote control 320″ receives input from a control interface 334i displayed on a display device 334 operated by the patient, the remote control 320″ transmits the control interface 334i in the form of a web-view, i.e. a remote interface that runs in a sandbox environment on the patient's display device 334. The patient's display device 334 can be, for example, a mobile phone, a tablet or a smart watch. In the embodiment shown in FIG. 20, the patient's display device 334 communicates with the remote control 320″ by means of BT. The control interface 334i in the form of a web-view is transmitted from the remote control 320″ to the patient's display device 334 over BT. Control commands in the form of inputs from the patient to the control interface 334i are transmitted from the patient's display device 334 to the remote control 320″ and provide input to the remote control 320″ equivalent to the input that may be provided using the control buttons 335. The control commands created in the patient's display device 334 are encrypted in the patient's display device 334 and transmitted to the remote control 320′ using BT.

The patient's display device 334 may (in the case of the display device 334 being a mobile phone or tablet) comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the remote control 320″. Disconnecting the auxiliary radio connections reduces the risk that the integrity of the control interface 334i displayed on the patient's display device 334 is compromised or that the control interface 334i displayed on the patient's display device 334 is remote controlled by an unauthorized device.

In alternative embodiments, control commands are generated and encrypted by the patient's display device and transmitted to the DDI 330. The DDI 330 can either alter the created control commands to commands readable by the remote control 320″ before further encrypting the control commands for transmission to the remote control 320″ or can add an extra layer of encryption before transmitting the control commands to the remote control 320″ or can simply act as a router for relaying the control commands from the patient's display device 334 to the remote control 320″. It is also possible that the DDI 330 adds a layer of end-to-end encryption directed at the implanted medical device MD, such that only the implanted medical device MD can decrypt the control commands to perform the command intended by the patient.

The patient's display device 334 can have a first and second application related to the implanted medical device MD. The first application is the control application displaying the control interface 334i for controlling the implanted medical device MD, whereas the second application is a general application for providing the patient with general information about the status of the implanted medical device MD or information from the DDI 330 or HCP or for providing an interface for the patient to provide general input to the DDI 330 or HCP related to the general well-being of the patient, lifestyle of the patient or general input from the patient concerning the function of the implanted medical device MD. The second application does not provide input to the remote control 320″ and/or the implanted medical device MD, thus handles data which are less sensitive. As such, the general application can be configured to function also when all auxiliary radio connections are activated, whereas switching to the control application which handles the more sensitive control commands and communication with the implanted medical device MD can require that the auxiliary radio connections are temporarily de-activated. It is also possible that the control application is a sub-application running within the general application, in which case the activation of the control application as a sub-application in the general application can require the temporary de-activation of auxiliary radio connections. In the embodiment shown in FIG. 20, access to the control application requires the use of optical and/or NFC means of a hardware key 333′ in combination with biometric input to the patient's display device, whereas accessing the general application only requires biometric input to the patient's display device and/or a pin code. In the alternative, a two-factor authentication solution, such as a digital key in combination with a pin code, can be used for accessing the general application and/or the control application.

In the embodiments in which the patient's display device 334 is configured to display and interact only with a web-view provided by another unit of the system, it is possible that the web-view is a view of a back-end provided on the DDI 330, and in such embodiments, the patient interacting with the control interface on the patient's display device is equivalent to the patient interacting with an area of the DDI 330.

Turning now to the P-EID 320″ “, the P-EID 320” is an external device which communicates with, and charges, the implanted medical device MD. The P-EID 320″ can be remotely controlled by the HCP to read information from the implanted medical device MD, control the operation of the implanted medical device MD, control the charging of the implanted medical device MD, and adjust the settings to the software running on the controller 300 of the implanted medical device MD, e.g. by adding or removing pre-defined program steps and/or by the selection of pre-defined parameters within a limited range. Just as the remote control 320″, the P-EID 320″” can be configured to communicate with the implanted medical device MD using BT or UWB communication. Just as with the remote control 320″, it is also possible to use a combination of UWB wireless communication and BT for enabling positioning of the P-EID 320″ as a way to establish that the P-EID 320″ is at a position which the implanted medical device MD and/or patient and/or HCP can acknowledge as being correct, e.g. in the direct proximity to the correct patient and/or the correct medical device MD. Just as for the remote control 320″, in embodiments in which a combination of BT and UWB technology is used, the UWB technology may be used for location-based authentication of the P-EID 320″, whereas the communication and/or data transfer can take place using BT. The P-EID 320″ comprises a wireless transmitter/transceiver 328 for communication and also comprises a wireless transmitter 325 configured for transferring energy wirelessly, in the form of a magnetic field, to a wireless receiver 395 of the implanted medical device MD configured to receive the energy in the form of a magnetic field and transform the energy into electric energy for storage in an implanted energy storage unit 40, and/or for consumption in an energy consuming part of the implanted medical device MD (such as the operation device, controller 300, etc.). The magnetic field generated in the P-EID and received in the implanted medical device MD is denoted a “charging signal”. In addition to enabling the wireless transfer of energy from the P-EID to the implanted medical device MD, the charging signal may also function as a means of communication. For instance, variations in the frequency of the transmission and/or amplitude of the signal may be used as a signaling means for enabling communication in one direction, from the P-EID to the implanted medical device MD, or in both directions between the P-EID and the implanted medical device MD. The charging signal in the embodiment shown in FIG. 20 is a signal in the range of 120 to 140 kHz, and the communication follows a proprietary communication signaling protocol, i.e. it is not based on an open standard. In alternative embodiments. BT can be combined with communication using the charging signal or communication using the charging signal can be combined with a UWB signal.

Just as for the remote control 320″, the UWB signal can in some embodiments also be used as a wake-up signal for the controller 300, or for the BT transceiver, such that the BT transceiver in the implanted medical device MD can be turned off when not in use, which eliminates the risk that the BT is intercepted or that the controller 300 of the implanted medical device MD is hacked by means of BT communication. In the alternative, the charging signal can be used as a wake-up signal for the BT, as the charging signal does not travel very far. Also, as a means of location-based authentication, the effect of the charging signal or the RSSI can be assessed by the controller 300 in the implanted medical device MD to establish that the transmitter is within a defined range. In the BT/UWB combination, the UWB may be used also for transmission of data. In some embodiments, the UWB and/or the charging signal can be used for the transmission of some portions of the data, such as sensitive portions of the data, or for the transmission of keys for unlocking encrypted communication sent by BT.

UWB can also be used for waking up the charging signal transmission, starting the wireless transfer of energy or initiating communication using the charging signal. As the signal for transferring energy has a very high effect in relation to normal radio communication signals, the signal for transferring energy cannot be active all the time, as this signal may be hazardous, e.g. by generating heat.

The P-EID 320′″ communicates with the HCP over the Internet by means of a secure communication, such as over a VPN. The communication between the HCP and the P-EID 320″″ is preferably encrypted. The communication from the HCP to the implanted medical device MD may be performed using an end-to-end encryption, in which case the communication cannot be decrypted by the P-EID 320″ “. In such embodiments, the P-EID 320”′ acts as a router which merely passes on encrypted communication from the HCP to the controller 300 of the implanted medical device MD. This solution further increases security as the key for decrypting the information rests only with the HCP and with the implanted medical device MD, which reduces the risk that an unencrypted signal is intercepted by an unauthorized device.

When the implanted medical device MD is to be controlled and/or updated remotely by the HCP via the P-EID 320″ “, an HCP Dedicated Device (DD) 332 displays an interface in which predefined program steps or setting values are presented to the HCP. The HCP provides input to the HCP DD 332 by selecting program steps, altering settings and/or values or altering the order in which pre-defined program steps are to be executed. The instructions/parameters put into the HCP DD 332 for remote operation is, in the embodiment shown in FIG. 20, routed to the P-EID 320″″ via the DDI 330 which may or may not be able to decrypt/read the instructions. The DDI 330 may store the instructions for a time period to later transfer the instructions in a package of created instructions to the P-EID 320″ “. It is also possible that an additional layer of encryption is provided to the package by the DDI 330. The additional layer of encryption may be a layer of encryption to be decrypted by the P-EID 330 or a layer of encryption which may be decrypted only by the controller 300 of the implanted medical device MD, which reduces the risk that unencrypted instructions or packages are intercepted by unauthorized devices. The instructions/parameters are then provided to the P-EID 320”, which then loads the instructions/parameters into the implanted medical device MD during the next charging/energy transfer using any of the signal transferring means (wireless or conductive) disclosed herein.

The Health Care Provider EID (HCP EID) 320′ has the same features as the P-EID 320″ and can communicate with the implanted medical device MD in the same alternative ways (and combinations of alternative ways) as the P-EID 320′″. However, in addition, the HCP EID 320′ also enables the HCP to freely re-program the controller 300 of the implanted medical device MD, including replacing the entire program code running in the controller 300. The idea is that the HCP EID 320′ always remains with the HCP and, as such, all updates to the program code or retrieval of data from the implanted medical device MD using the HCP EID 320′ is performed with the HCP being present (i.e. not remote). The physical presence of the HCP is an additional layer of security for these updates which may be critical to the function of the implanted medical device MD.

In the embodiment shown in FIG. 20, the HCP communicates with the HCP EID 320′ using the HCP Dedicated Device 332 (HCP DD), which is a display device comprising a control interface for controlling and communicating with the HCP EID 320′. As the HCP EID 320′ always stays physically at the HCP's clinic, communication between the HCP EID 320′ and HCP DD 332 does not have to be sent over the Internet. Instead, the HCP DD 332 and the HCP EID 320′ can communicate using one or more of BT, a proprietary wireless communication channel and a wired connection. Then the alteration to the programming is sent to the implanted medical device MD directly via the HCP EID 320′. Inputting into the HCP DD 332 for direct operation by means of the HCP EID 320′ is the same as inputting directly into the HCP EID 320′, which then directly transfers the instructions into the implanted medical device MD.

In the embodiment shown in FIG. 20, both the patient and the HCP have a combined hardware key 333″, 333″. The combined key 333′, 333″ comprises a hardware component comprising a unique circuitry (providing the highest level of security), a wireless NFC transmitter 339 for transmitting a specific code (providing mid-level security), and a printed QR code 344 for optical recognition of the card (providing the lowest level of security).

The patient's key 333′ in the embodiment shown in FIG. 20 is in the form of a key card having an interface for communicating with the P-EID 320′″ such that the key card can be inserted into a key card slot in the P-EID 320″. The NFC transmitter 339 and/or the printed QR code 344 can be used as means for accessing the control interface 334i of the display device 334. In addition, the display device 334 may require a pin-code and/or a biometric input, such as face recognition or fingerprint recognition.

The HCP's key 333″ in the embodiment shown in FIG. 20 is in the form of a key card having an interface for communicating with the HCP-EID 320′, such that the key card can be inserted into a key card slot in the HCP-EID 320′. The NFC transmitter 339 and/or the printed QR code 344 can be used as means for accessing the control interface of the HCP DD 332. In addition, the HCP DD 332 may require a pin-code and/or a biometric input, such as face recognition or fingerprint recognition.

In alternative embodiments, it is however possible that the hardware key solution is replaced by a two-factor authentication solution, such as a digital key in combination with a PIN code or a biometric input (such as face recognition and/or fingerprint recognition).

In the embodiment shown in FIG. 20, communication over the Internet takes place over the Dedicated Data Infrastructure (DDI) 330 which runs on a cloud service. The DDI 330 handles communication between the HCP DD 332 and the P-EID 320″ “, between the HCP and the remote control 320”, between the HCP and the patient's display device 334, as well as between the HCP and auxiliary devices 336 (such as tools for following up the patient's treatments, e.g. a scale in an obesity treatment example or a blood pressure monitor in a blood pressure treatment example). In some embodiments, the HCP DD 332 also handles the communication between the patient's display device 334 and the remote control 335. In all examples, the communication from the HCP to the P-EID 320″ **, remote control 320″, patient's display device 334 and auxiliary devices 336 may be performed using an end-to-end encryption. In embodiments with end-to-end encryption, the communication cannot be decrypted by the DDI 330. In such embodiments, the DDI 330 acts as a router which merely passes on encrypted communication from the HCP to various devices. This solution further increases security as the keys for decrypting the information rests only with the HCP and with the device sending or receiving the communication, which reduces the risk that an unencrypted signal is intercepted by an unauthorized device.

In addition to acting as an intermediary or router for communication, the DDI 330 collects data of the implanted medical device MD, of the treatment and of the patient. The data may be collected in an encrypted, anonymized or open form. The form of the collected data may depend on the sensitivity of the data or on the source from which the data is collected. In the embodiment shown in FIG. 20, the DDI 330 sends a questionnaire to the patient's display device 334. The questionnaire can comprise questions to the patient related to the general health of the patient, related to the way of life of the patient, or specifically related to the treatment provided by the implanted medical device MD (such as for example a visual analogue scale for measuring pain). The DDI 330 can compile and/or combine input from several sources and communicate such input to the HCP which can use the provided information to create instructions to the various devices to be sent back over the DDI 330. The data collection performed by the DDI 330 can also be in the form a log to make sure that all communication between the units in the system can be back-traced. Logging the communication ensures that all alterations to software or settings of the software as well as the frequency and operation of the implanted medical device MD can be followed. Following the communication enables the DDI 330 or the HCP to follow the treatment and react if something in the communication indicates that the treatment does not provide the intended results or if something appears to be wrong with any of the components in the system.

In the specific embodiment shown in FIG. 20, the wireless connections between the different units are as follows. The wireless connection 411 between the auxiliary device 336 and the DDI 330 is based on Wi-Fi or a mobile telecommunication regime, and the wireless connection 411 between the auxiliary device 336 and the patient's display device 334 is based on BT. The wireless connection 412 between the patient's display device 334 and the DDI 330 is based on Wi-Fi or a mobile telecommunication regime. The wireless connection 413 between the patient's display device 334 and the remote control 320″ is based on BT. The wireless connection 414 between the remote control 320″ and the implanted medical device MD is based on BT and UWB. The wireless connection 415 between the remote control 320″ and the DDI 330 is based on Wi-Fi or a mobile telecommunication regime. The wireless connection 416 between the P-EID 320″” and the implanted medical device MD is based on BT. UWB and the charging signal. The wireless connection 417 between the P-EID 320″> and the DDI 330 is based on Wi-Fi or a mobile telecommunication regime. The wireless connection 418 between the HCP-EID 320′ and the implanted medical device MD is based on BT. UWB and the charging signal. The wireless connection 419 between the P-EID 320″” and the HCP DD 332 is based on BT. The wireless connection 420 between the HPC-EID 320′ and the DDI 330 is based on Wi-Fi or a mobile telecommunication regime. The wireless connection 421 between the HPC DD 332 and the DDI 330 is based on Wi-Fi or a mobile telecommunication regime. The wireless connection 422 between the HCP-EID 320′ and the HCP DD 332 is based on BT.

The wireless connections specifically described in the embodiment shown in FIG. 20 may, however, be replaced or assisted by wireless connections based on radio frequency identification (RFID), near-field communication (NFC). Bluetooth. Bluetooth low energy (BLE), or wireless local area network (WLAN). The mobile telecommunication regimes may for example be IG. 2G. 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and ultra-high frequency band (UHF) as well as essentially any other applicable band for electromagnetic wave communication. The wireless connection may also be based on ultrasound communication to name at least one example that does not rely on electromagnetic waves.

Although wireless transfer is primarily described in the embodiment disclosed with reference to FIG. 20, the wireless communication between any of the external device may be substituted for wired communication. Also, some or all of the wireless communication between an external device and the implanted medical device MD may be substituted for conductive communication using a portion of the human body as a conductor (such as further described with reference to FIGS. 16A to 16C).

### General Communication Housing

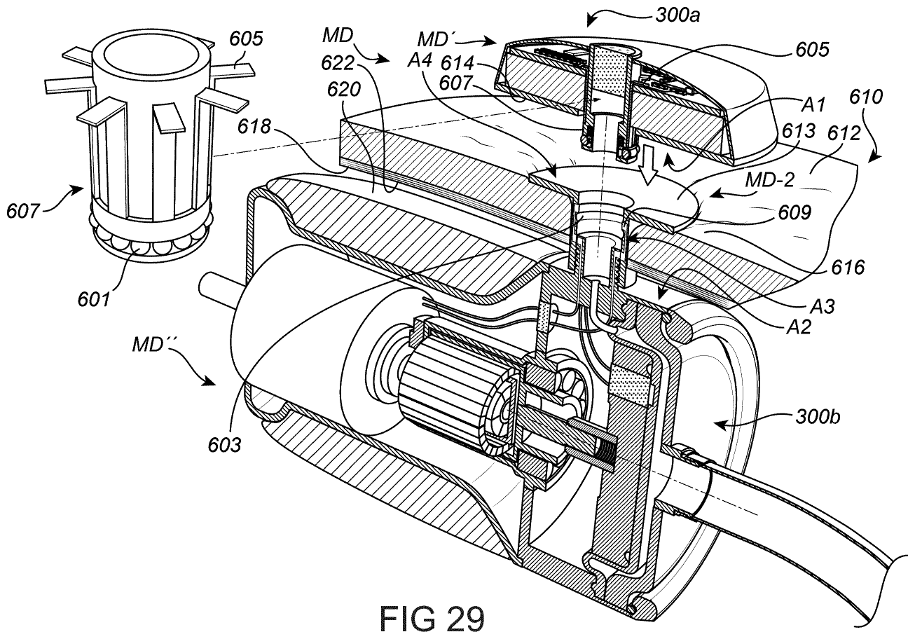

As have been discussed before in this application, communication with a medical implant needs to be reliable and secure. For this purpose, it is desirable to have a standalone device as an external remote control (for example described as 320″ in FIG. 20 for the medical implant, such that no other programs or applications run on the same device which may disturb or corrupt the communication to the medical implant. However, the smartphone or tablet (for example described as 334 in FIGS. 119a-119h) has become an integrated part of everyday life for most people. This means that we almost always have our smartphones at hand. For this reason, it would have been convenient for the patient to communicate with the medical implant directly using the smartphone, such that no additional standalone device would have to be carried. However, as a lot of other applications are running on the smartphone, it does not fulfill the requirement of being a secure and reliable communication tool without interference from other communication. It is therefore desirable to split the tasks of providing secure communication between the external device and the implant from the task of communicating with the Internet and providing a familiar and intuitive user interface. For this purpose, and external device providing secure communication and tamperproof soft—and hardware, where the display device allows for intuitive and easy use is provided. In the embodiments described with reference to FIGS. 21-25 a device fulfilling these combinatory needs will be described in the form of a standalone remote control external device integrated in a housing unit 320″ connectable to a smartphone or another display device 334, such as a smart watch or a tablet.

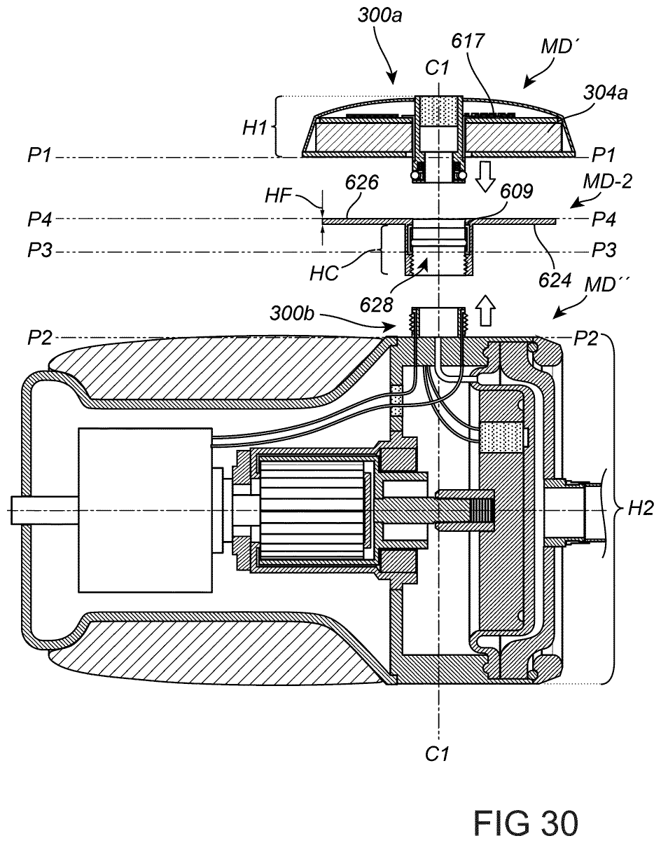

FIG. 21 shows the housing unit 320″ in an elevated perspective view form the left, and FIG. 22 shows the housing unit 320″ in a plain view from the left. In the embodiment shown in FIG. 21, the housing unit 320″ has a rectangular shape with rounded edges, having a height 1521 which is more than 1.5 times the width 1522. The housing unit 320″ comprises recess 1525 configured to receive a display device 334, in the form of a smartphone, configured to be fitted in the housing unit 320″ for mechanically and disconnectably connecting the display device 334 to the housing unit 320″. The boundaries of the recess 1525 in the housing unit 320″ forms an edge 1528 configured to encircle the display device 334, when the display device 334 is inserted into the recess 1525. In the embodiment shown in FIG. 21, the recess 1525 has a depth 1526 configured to allow the display device 334 to be entirely inserted into the recess 1525. As such, the depth 1526 of the recess 1525 exceeds the depth 1531 of the display device 334. In the embodiment shown in FIGS. 21 and 22, the edge is relatively thin, and has a width 1527 which is in the range ⅛- 1/100 of the width of the display device 334, as such, the housing unit 320″ has a width in the range 1.02-1.25 times the width 1522 of the housing unit 320″. In the same way, the housing unit 320″ has a height 1521 in the range 1.01-1.25 times the height 1521 of the display device 334. In the embodiment shown in FIGS. 120-22, the edges 1528 are configured to clasp the display device 334 and thereby mechanically fixate the display device 334 in the housing unit 320″. The minimum bounding box of the housing unit 320″ and the display device 334 when mechanically connected, is no more than. 10% wider. 10% longer or 100% higher, than the minimum bounding box of the display device 334.

For creating a clasping fixation, the edges of the housing unit 320″ is made from an elastic material crating a tension between the edge 1528 and the display device 334 holding the display device 334 in place. The elastic material could be an elastic polymer material, or a thin sheet of elastic metal. For the purpose of further fixating the display device 334 in the housing unit 320″, the inner surface of the edges 1528 may optionally comprise a recess or protrusion (not shown) corresponding to a recess or protrusion of the outer surface of the display device 334. The edges 1528 may in the alterative comprise concave portions for creating a snap-lock clasping mechanical fixation between the housing unit 320″ and the display device 334.

In the embodiment shown in FIGS. 21 and 22, the housing unit 320″ functions as a remote control for communicating with an implanted medical device, including receiving information from, and providing instructions and updates to, the implanted medical device. Information could be information related to a state of the implanted medical device including any functional parameter of the implanted medical device or could be related to a state of the patient, including any physiological parameter pertaining to the body of the patient (further described on other sections of this disclosure). For the purpose of providing input to the implanted medical device and controlling and updating the functions of the housing unit 320″, the housing unit 320″ comprises a control interface comprising switches in the form of control buttons 335. The control buttons 335 are configured to be used when the external device is disconnected from the display device 334. The control interface further comprises a display 1505, which is a smaller and typically less sophisticated display 1505 than the display of the display device 334. In an alternative embodiment, the control buttons 335 and display 1505 are integrated into a single touch-responsive (touchscreen) display on which the control buttons may be displayed. In the embodiment shown in FIGS. 21 and 22, one of the control buttons 335 is a control button for activating the implanted medical device and another of the control buttons 335 is a control button for deactivating the implanted medical device. When the display device 334 is attached to the housing unit 320″, the control buttons 335 and the display is covered by the display device 334 and are as such not in an operational state. In the embodiment shown in FIGS. 21 and 22, the housing unit 320″ is configured to transmit information pertaining to the display of the user interface to the display device 334 and the display device 334 is configured to receive input pertaining to communication to or from the implantable medical device from the patient, and transmit signals based on the received input to the housing unit 320″. The input may be a command to change the operational state of the implantable medical device. The display device 334 comprises a touch screen configured to display the user interface and receive the input from the patient. The display of the display device 334 may comprise one or more OLEDs or IPS LCDs elements. When the display device 334 is connected to the housing unit 320″, the display device 334 is configured to display a control interface which is used to communicate with the housing unit 320″, i.e. providing input to and receiving information from the housing unit 320″. The input provided the housing unit 320″ is then relayed to the implanted medical device—and in the same way information communicated from the implanted medical device to the housing unit 320″ may be relayed or displayed on the display device 334. Having an external device comprising a combination of a housing unit 320″ comprising the communication means for communicating with the implanted medical device and a display device 334 basically only functioning as an Input/Output device connected to the housing unit 320″ makes it possible to have a secure communication between the housing unit 320″ and the display device 334, which is out of reach from the Internet connection of the display device 334, which makes it much harder for an external attacker to get access to any of the vital communication portions of the housing unit 320″. The communication between the housing unit and the display device 334 is very restricted and the only communication allowed from the display device 334 to the housing unit 320″ is input from the patient or a healthcare professional, and authentication parameters created by an authentication application running on the display device 334. The authentication application running on the display device 334 could be a number-generating authenticator or a biometric authenticator for authenticating the patient or health care professional, and the authentication parameters could for example be parameters derived from a facial image or a fingerprint. In the opposite direction, i.e. from the housing unit 320″ to the display device 334, the communication could be restricted to only communication needed for displaying information and/or a graphical user interface on the display device 334. The communication restrictions could for example be based on size of the communication packages or the frequency with which the communication takes place which reduces the risk that an un-authorized person makes multiple attempts to extract information from, or transit information to, the hand-held device.

In the embodiment shown with reference to FIGS. 21 and 22, the housing unit 320″ comprises a first communication unit providing a wireless connection 413 to the display device 334. The wireless connection 413 is in the embodiment shown in FIGS. 21 and 22 based on NFC, but could in alternative embodiment be based on Bluetooth or any other communication pathway disclosed herein. The housing unit 320″ further comprises a second communication unit providing a wireless connection with the implanted medical device. The wireless communication between the housing unit 320″ and the implanted medical device is in the embodiment shown in FIGS. 21 and 22 based on Bluetooth, but could in alternative embodiments be based on NFC or UWB or any other communication pathway disclosed herein.

As mentioned, in the embodiment shown in FIGS. 21 and 22, the wireless communication between the housing unit 320″ and the display device 334 is based on NFC, while the wireless communication between the housing unit 320″ and the is based on Bluetooth. As such, the first communication unit of the housing unit 320″ is configured to communicate wirelessly with the display device 334 using a first communication frequency and the second communication unit of the housing unit 320″ is configured to communicate wirelessly with the implantable medical device using a second different communication frequency. For this purpose, the first communication unit of the housing unit 320″ comprises a first antenna configured for NFC-based wireless communication with the display device 334, and the second communication unit comprises a second antenna configured for Bluetooth-based wireless communication with the implantable medical device. The first and second antennae may be a wire-based antennae or a substrate-based antennae. As such, the first communication unit is configured to communicate wirelessly with the display device 334 on a first frequency and the second communication unit is configured to communicate wirelessly with the implantable medical device using a second different communication frequency. Also, first communication unit of the housing unit 320′ is configured to communicate wirelessly with the display device 334 using a first communication protocol (the NFC-communication protocol), and the second communication unit is configured to communicate wirelessly with the implantable medical device using a second communication protocol (the Bluetooth communication protocol). The first and second communication protocols are different which adds an additional layer of security as security structures could be built into the electronics and/or software enabling the transfer from a first to a second communication protocol.

In an alternative embodiment, the second communication unit may be configured to communicate wirelessly with the implantable medical device using electromagnetic waves at a frequency below 100 kHz, or preferably at a frequency below 40 kHz. The second communication unit may thus be configured to communicate with the implantable medical device using “Very Low Frequency” communication (VLF). VLF signals have the ability to penetrate a titanium housing of the implant, such that the electronics of the implantable medical device can be completely encapsulated in a titanium housing. In yet further embodiments, the first and second communication units may be configured to communicate by means of an RFID type protocol, a WLAN type protocol, a BLE type protocol, a 3G/4G/5G type protocol, or a GSM type protocol.

In yet other alternative embodiments, it is conceivable that the mechanical connection between the housing unit 320″ and the display device 334 comprises an electrical connection for creating a wire-based communication channel between the housing unit 320″ and the display device 334. The electrical connection could also be configured to transfer electric energy from the display device 334 to the housing unit, such that the housing unit 320″ may be powered or charged by the display device 334. A wired connection is even harder to access for a non-authorized entity than an NFC-based wireless connection, which further increases the security of the communication between the housing unit 320″ and the display device 334.

In the embodiment shown with reference to FIGS. 21 and 22, the display device 334 comprises a first communication unit providing a wireless connection 413 to the housing unit 320″ based on NFC. The display device 334 further comprises a second communication unit providing a wireless connection with a further external device and/or with the Internet. The second external device may be far away, for example at a hospital or a place where a medical professional practice. The wireless communication between the display device 334 and a further external device is in the embodiment shown in FIGS. 21 and 22 based on WiFi, but could in alternative embodiments be based on for example Bluetooth.

As mentioned, in the embodiment shown in FIGS. 21 and 22, the wireless communication between the display device 334 and the housing unit 320″ is based on NFC, while the wireless communication between the display device and a further external unit is based on WiFi. As such, the first communication unit of the display device 334 is configured to communicate wirelessly with the housing unit 320″ using a first communication frequency and the second communication unit of the display device 334 is configured to communicate wirelessly with a further external device using a second different communication frequency. For this purpose, the first communication unit of the display device 334 comprises a first antenna configured for NFC-based wireless communication with the housing unit 320″, and the second communication unit comprises a second antenna configured for WiFi-based wireless communication with a further external device. The first and second antennae may be wire-based antennae or substrate-based antennae. As such, the first communication unit is configured to communicate wirelessly with the housing unit 320″ on a first frequency and the second communication unit is configured to communicate wirelessly with the further external device using a second different communication frequency. Also, the first communication unit of the display device 334 is configured to communicate wirelessly with the housing unit 320″ using a first communication protocol (the NFC communication protocol), and the second communication unit is configured to communicate wirelessly with the further external device using a second communication protocol (the WiFi communication protocol). The first and second communication protocols are different which adds an additional layer of security as security structures could be built into the electronics and/or software enabling the transfer from a first to a second communication protocol.

In alternative embodiments, the second communication unit of the display device 334 may be configured to communicate with the further external device by means of, a WLAN-type protocol, or a 3G/4G/5G-type protocol, or a GSM-type protocol.

In the embodiment shown in FIGS. 21 and 22, the communication range of the first communication unit of the housing unit 320″ is less than a communication range of the second communication unit of the housing unit 320′, such that the communication distance between the housing unit 320″ and the medical implant may be longer than the communication distance between the housing unit 320″ and the display device 334. In the embodiment shown in FIGS. 21 and 22, the communication range of the first communication unit may be constrained to a length that is less than five times the longest dimension of the minimal bounding box of the display device 334, or more precisely constrained to a length that is less than three times the longest dimension of the minimal bounding box of the display device 334.

In the embodiment shown in FIGS. 21 and 22, communication between the housing unit 320″ and the display device 334 is only enabled when the housing unit 320″ is connected to the display device 334. I.e, at least one of the housing unit 320″ and the display device 334 is configured to allow communication between the housing unit 320″ and the display device 334 on the basis of the distance between the housing unit 320″ and the display device 334. In the alternative, the housing unit 320″ and/or the display device 334 may comprise a sensor configured to estimate whether the housing unit 320″ is attached to the display device 334 or not, such as a mechanically activated switch or a photo resistive sensor which providing sensor input when the housing unit 320″ and display device 334 are mechanically connected to each other. The signal from the at least one sensor then may be used to permit usage of the communication unit configured for communication with the display device 334.

In the embodiment shown in FIGS. 21 and 22, communication between the housing unit 320″ and the implantable medical device is only enabled on the basis of a distance between the housing unit 320″ and the implantable medical device. In the embodiment shown in FIGS. 21 and 22, the distance should be less than twenty times the longest dimension of the minimal bounding box of the display device, or more specifically less than ten times the longest dimension of the minimal bounding box of the display device. The distance between the housing unit 320″ and the medical implant may be measured using electromagnetic waves, or acoustic waves. The process of measuring the distance may comprise triangulation.

In the embodiment shown in FIGS. 21 and 22, the second communication unit of the display device 334 need to be disabled to enable communication between the display device 334 and the housing unit 320″, and further the second communication unit of the display device 334 needs to be disabled to enable communication between the housing unit 320″ and the medical implant. Also, the second communication unit of the housing unit 320″ needs to be disabled to enable communication between the housing unit 320″ and the medical implant.

In the embodiment shown in FIGS. 21 and 22, the housing unit 320″ further comprises an encryption unit configured to encrypt communication received from the display device 334 before transmitting the communication to the implanted medical device. The encryption unit may for example be based on one of the following algorithms: AES, Blowfish, DES, Kalyna, Serpent or Twofish. For the purpose for handling the communication, I/O and encryption, the housing unit 320″ comprises a processor which could be a general-purpose microprocessor and/or an instruction set processor and/or related chips sets and/or special purpose microprocessors such as ASICs (Application Specific Integrated Circuit). The processor also comprise memory for storing instruction and/or data.

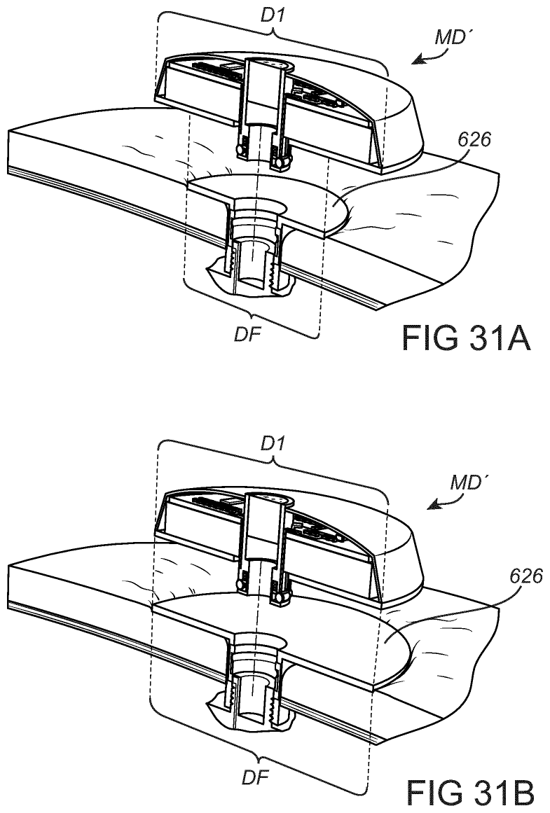

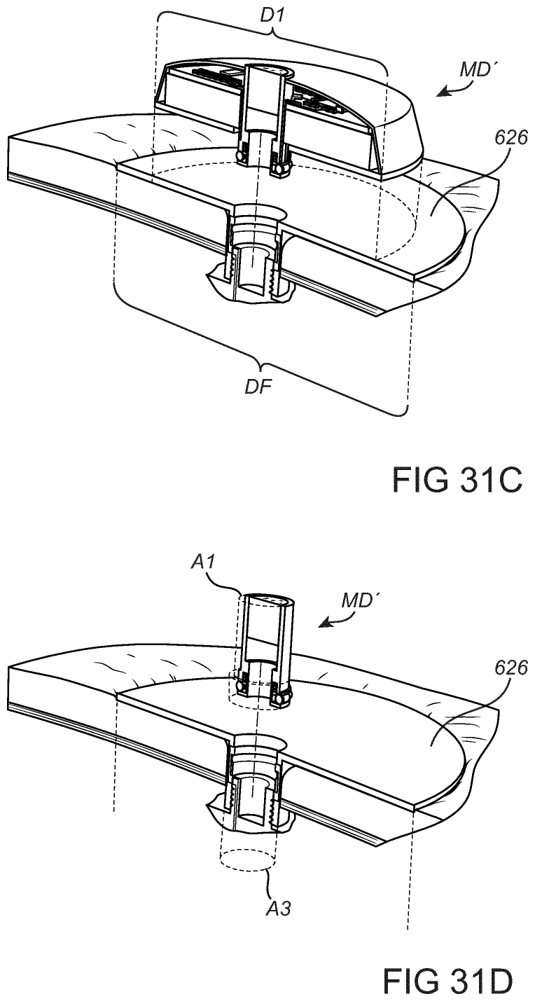

FIGS. 23 and 24 shows an embodiment of the external unit similar to the embodiment described with reference to FIGS. 21 and 22. The difference being that in the embodiment of FIGS. 23 and 24, the housing unit 320″ does not clasp the display device 334. Instead, the housing unit comprises two magnets 1510 for magnetically fixating the display device 334 to the housing unit 320″. In alternative embodiments, it is equally conceivable that the external device comprises an intermediate portion, which is fixedly fixated to the housing unit for providing a detachable connection with the display device 334. In the alternative, the intermediate device could be fixedly fixated to the display device 334 and provide a detachable connection with the housing unit 320″.

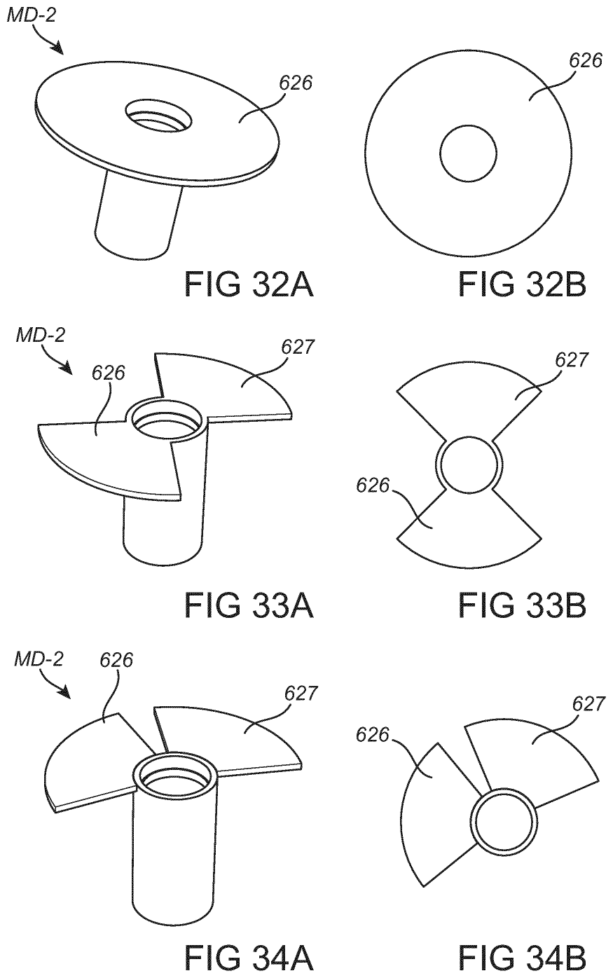

FIG. 25 shows a system overview of the external device (which could be the external device of the embodiment described with reference to FIGS. 21 and 22, or of the embodiment described with reference to FIGS. 23 and 24). The housing unit 320″ is connected to the display device 334. A wireless connection 413 is provided between the housing unit 320″ and the display device 334, and a further wireless connection 413 is provided between the housing unit 320″ and the implanted medical device MD, such that the housing unit can send instructions and updates to the implanted medical device MD, and receive information, parameters (such as sensor values) and alarms from the implanted medical device MD. The communication between the external device and the medical implant MD is further described in other portions of this disclosure.

### Surface Coatings

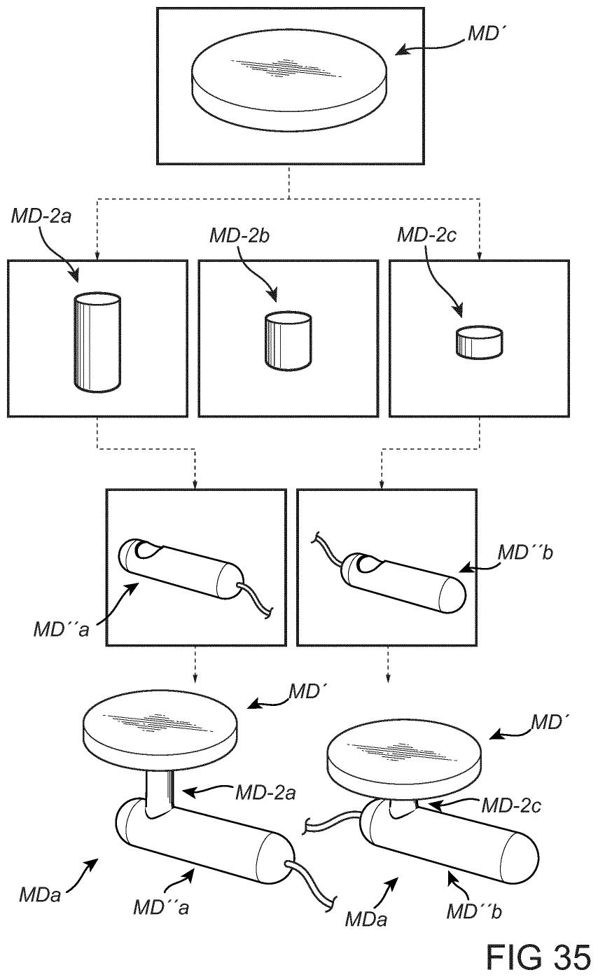

FIG. 26 shows an implantable medical device or implant MD comprising a body 510, an implant surface 520 and a coating 530 arranged on the surface 520. The coating 530 may be configured to have antibacterial characteristics. Depending on the use of the implantable medical device, one or both of these effects may be advantageous. The coating 530 may be arranged on the surface 520 so that the coating shields the surface 520 from direct contact with the host body where the implantable medical device MD is inserted.

The coating 530 may comprise at least one layer of a biomaterial. The coating 530 may comprise a material that is antithrombotic. The coating 530 may also comprise a material that is antibacterial. The coating 530 may be attached chemically to the surface 520.

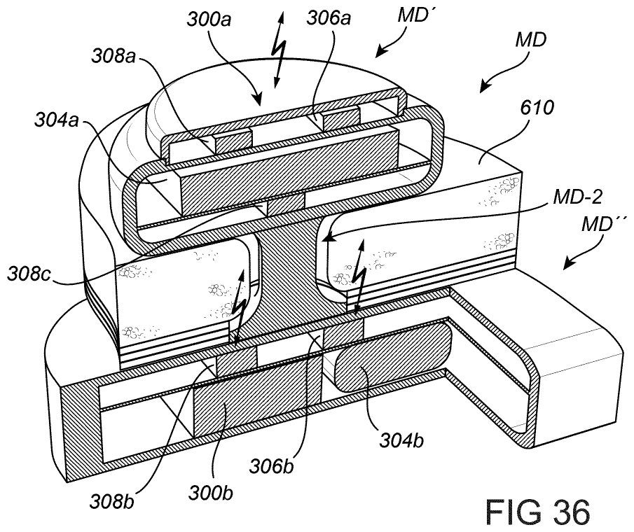

FIG. 27 shows an exemplary implantable medical device or implant MD with a body 510 and a surface 520. The implantable medical device MD comprises multiple coatings, 530a, 530b, 530c arranged on the surface. The implant MD may comprise any number of coatings, the particular embodiment of FIG. 6 discloses three layers of coating 530a, 530b, 530c. The second coating 530b is arranged on the first coating 530a. The different coatings 530a, 530b, 530c may comprise different materials with different features to prevent either fibrin sheath formation or bacteria gathering at the surface 520. As an example, the first coating 530a may comprise a layer of perfluorocarbon chemically attached to the surface. The second coating 530b may comprise a liquid perfluorocarbon layer arranged on the first coating 530a.

The coatings referred to may comprise any substance or any combination of substances. The coatings may comprise anticoagulant medicaments, such as: Apixaban, Dabigatran, Dalteparin, Edoxaban, Enoxaparin, Fondaparinux, Heparin, Rivaroxaban, and Warfarin.

The coatings may also comprise medicines or substances that are so-called antiplatelets. These may include Aspiring. Cilostazol, Clopidogrel, Dipyridamole, Eptifibatide, Prasugrel, Ticagrelor, Tirofiban, Vorapaxar.

The coatings may also comprise any other type of substance with antithrombotic, antiplatelet or antibacterial features, such as sortase A, perfluorocarbon and more.

The coatings may also be combined with an implantable medical device comprising certain materials that are antibacterial or antithrombotic. For example, some metals have shown to be antibacterial. In case the implant or at least the outer surface of the implant is made of such a metal, this may be advantageous in order to reduce bacterial infections. The medical implant or the surface of the implant may be made of any other suitable metal or material. The surface may for example comprise any of the following metals or any combination of the following metals: titanium, cobalt, nickel, copper, zinc, zirconium, molybdenum, tin or lead.

An implantable medical device can also be coated with a local and slow-releasing anti-fibrotic or antibacterial drug in order to prevent fibrin sheath creation and bacterial inflammation. The drug or medicament may be coated on the surface and arranged to slowly release from the implant in order to prevent the creation of fibrin or inflammation. The drug may also be covered in a porous or soluble material that slowly disintegrates in order to allow the drug to be administered into the body and prevent the creation of fibrin. The drug may be any conventional anti-fibrotic or antibacterial drug.

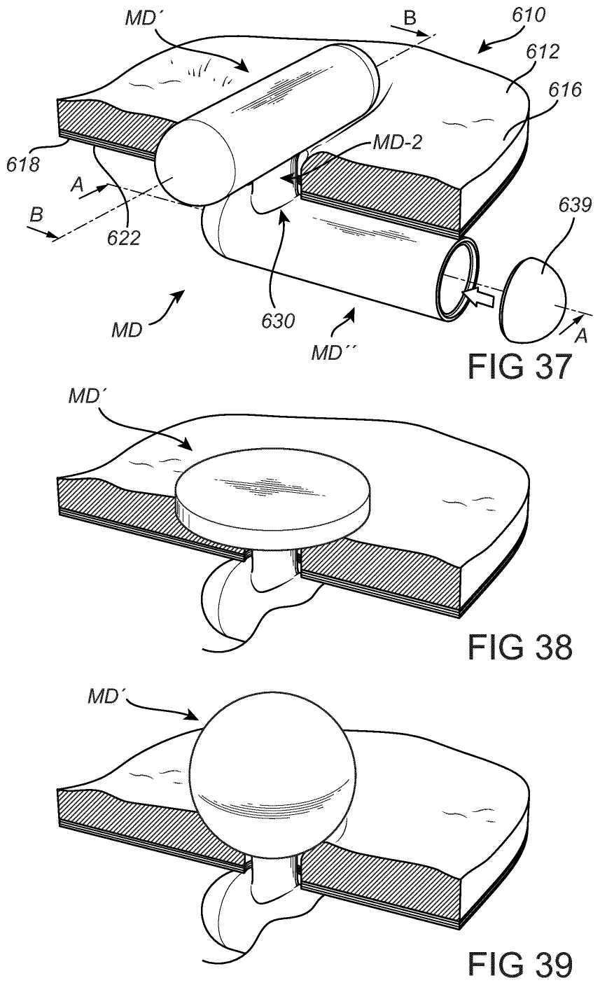

FIGS. 28A and 28B show different micro patterns on the surface of an implant. In order to improve blood compatibility, the implant material's physical structure may be altered or controlled. By creating a certain topography on the surface of an implant, fibrin creation and inflammatory reactions may be inhibited. FIG. 28A is an example of a micro pattern that mimics the features of sharkskin. The micro pattern may have many different shapes and many different depths into the surface of the implant and they may be a complement to other coatings or used individually. In FIG. 28B another example of a micro pattern is disclosed.

The micro pattern may, for example, be etched into the surface of the implantable medical device prior to insertion into the body. The surface of the implantable medical device may for example comprise a metal. The surface may for example comprise any of the following metals, or any combination of the following metals: titanium, cobalt, nickel, copper, zinc, zirconium, molybdenum, tin or lead. This may be advantageous in that these metals have proven to be antibacterial which may ensure that the implant functions better when inserted into the host body.

### Pop Rivet Flange

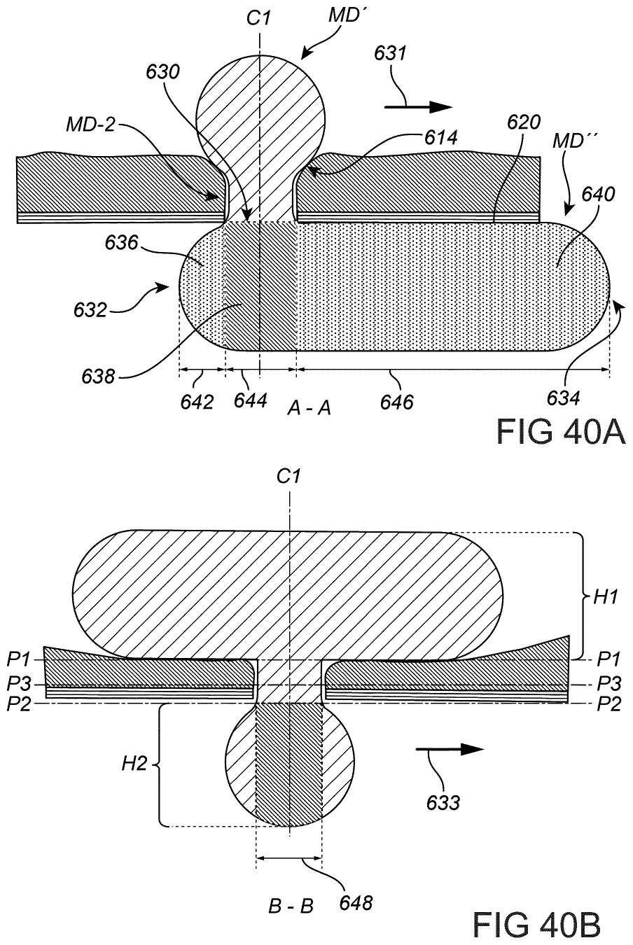

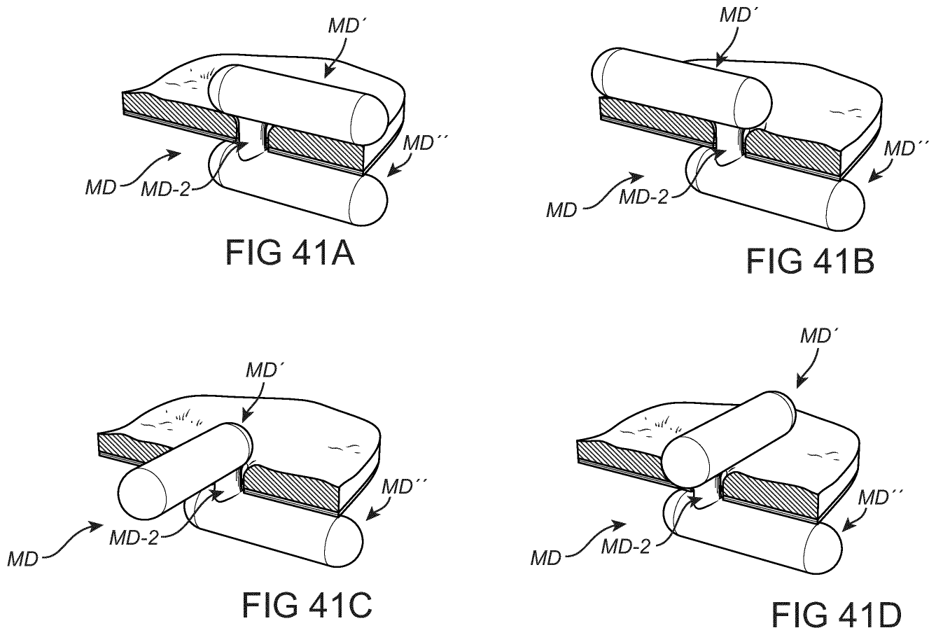

FIGS. 29 and 30 show an embodiment of an implantable energized medical device MD, which may be referred to as a remote unit in other parts of the present disclosure. The medical device MD is configured to be held in position by a tissue portion 610 of a patient. The first portion MD′ and the second portion MD″ may comprise one or several functional parts, such as receivers, transmitters, transceivers, control units, processing units, sensors, energy storage units, etc., as is described in other parts of the present disclosure. The first portion MD′ may comprise a first energy storage unit for supplying the medical device MD with energy. While the second portion MD″ in the illustrated embodiment comprises a pump, this is just to give an example of an implantable part of a medical device MD. It is to be understood that other embodiments of the second portion MD″ can be connected to the first portion MD′ via the connecting portion MD-2, such as second portions MD″ comprising a motor for providing mechanical work without the use of fluids.

The medical device MD comprises a first portion MD′ configured to be placed on a first side 612 of the tissue portion 610, the first portion MD having a first cross-sectional area A1 in a first plane P1 and comprising a first surface 614 configured to face a first tissue surface 616 of the first side 612 of the tissue portion 610. The medical device MD further comprises a second portion MD″ configured to be placed on a second side 618 of the tissue portion 610, the second side 618 opposing the first side 612, the second portion MD″ having a second cross-sectional area A2 in a second plane P2 and comprising a second surface 620 configured to engage a second tissue surface 622 on the second side 618 of the tissue portion 610. The medical device MD further comprises a connecting portion MD-2 configured to be placed through a hole in the tissue portion 610 extending between the first and second sides 612, 618 of the tissue portion 610. The connecting portion MD-2 here has a third cross-sectional area A3 in a third plane P3 and a fourth cross-sectional area A4 in a fourth plane P4 and a third surface 624 configured to engage the first tissue surface 616 of the first side 612 of the tissue portion 610. The connecting portion MD-2 is configured to connect the first portion MD′ to the second portion MD″.

The connecting portion MD-2 thus has a portion being sized and shaped to fit through the hole in the tissue portion 610, such portion having the third cross-sectional area A3. Furthermore, the connecting portion MD-2 may have another portion being sized and shaped to not fit through the hole in the tissue portion 610, such portion having the fourth cross-sectional area A4. Likewise, the second portion MD″ may have a portion being sized and shaped to not fit through the hole in the tissue portion 610, such portion having the second cross-sectional area A2. Thus, the connecting portion MD-2 may cooperate with the second portion MD″ to keep the medical device MD in place in the hole of the tissue portion 610.

In the embodiment illustrated in FIG. 29, the first portion MD′ is configured to detachably connect, i.e. reversibly connect, to the connecting portion MD-2 by a mechanical and/or magnetic mechanism. In the illustrated embodiment, a mechanic mechanism is used, wherein one or several spring-loaded spherical elements 601 lock in place in a groove 603 of the connecting portion MD-2 when the first portion MD′ is inserted into the connecting portion MD-2. Other locking mechanisms are envisioned, including corresponding threads and grooves, self-locking elements, and twist—and—lock fittings.

The medical device MD is configured such that, when implanted, the first portion MD′ will be placed closer to the outside of the patient than the second portion MD″. Furthermore, in some implantation procedures the medical device MD may be implanted such that space will be available beyond the second portion MD″, i.e. beyond the second side 618 of the tissue portion 610, whereas there may be as much space on the first side 612 of the tissue portion. Furthermore, tissue and/or skin may exert a force on the first portion MD′ towards the tissue portion 610 and provide that the second portion MD″ does not travel through the hole in the tissue portion 610 towards the first side 612 of the tissue portion 610. Thus, it is preferable if the medical device MD is primarily configured to prevent the first portion MD′ from traveling through the hole in the tissue portion 610 towards the second side 618 of the tissue portion 610.

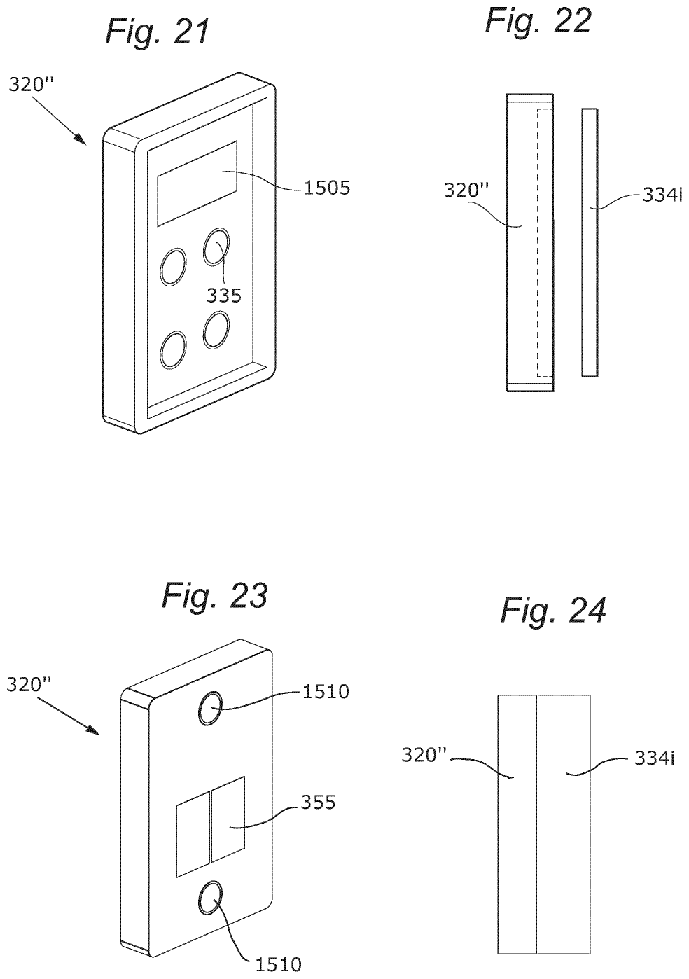

The first portion MD′ may further comprise one or several connections 605 for transferring energy and/or communication signals to the second portion MD″ via the connecting portion MD-2. The connections 605 in the illustrated embodiment are symmetrically arranged around a circumference of a protrusion 607 of the first portion MD′ and are arranged to engage with a corresponding connection 609 arranged at an inner surface of the connecting portion MD-2. The protrusion 607 may extend in a central extension C1 of the central portion MD-2. The second portion MD″ may also comprise one or several connections 611, which may be similarly arranged and configured as the connections 605 of the first portion MD′. For example, the one or several connections 611 may engage with the connection 609 of the connecting portion MD-2 to receive energy and/or communication signals from the first portion MD′. Although the protrusion 607 is illustrated separately in FIG. 18, it is to be understood that the protrusion 607 may be formed as one integral unit with the first portion MD″.

Other arrangements of connections are envisioned, such as asymmetrically arranged connections around the circumference of the protrusion 607. It is also envisioned that one or several connections may be arranged on the first surface 614 of the first portion MD″, wherein the connections are arranged to engage with corresponding connections arranged on the opposing surface 613 of the connecting portion MD-2. Such connections on the opposing surface 613 may cover a relatively large area as compared to the connection 609, thus allowing a larger area of contact and a higher rate and/or signal strength of energy and/or communication signal transfer. Furthermore, it is envisioned that a physical connection between the first portion MD′, connecting portion MD-2 and second portion MD″ may be replaced or accompanied by a wireless arrangement, as described further in other parts of the present disclosure.

Any of the first surface 614 of the first portion MD′, the second surface 620 of the second portion MD′, the third surface 624 of the connecting portion MD-2, and an opposing surface 613 of the connecting portion MD-2, may be provided with at least one of ribs, barbs, hooks, a friction-enhancing surface treatment, and a friction-enhancing material, to facilitate the medical device MD being held in position by the tissue portion and/or to facilitate that the different parts of the medical device MD are held in mutual position.

The opposing surface 613 may be provided with a recess configured to house at least part of the first portion MD′. In particular, such recess may be configured to receive at least a portion of the first portion MD′, including the first surface 614. Similarly, the first surface 614 may be provided with a recess configured to house at least part of the connecting portion MD-2. In particular, such recess may be configured to receive at least a portion of the connecting portion MD-2, and in some embodiments such recess may be configured to receive at least one protruding element to at least partially enclose at least one protruding element or flange.

In the illustrated embodiment, the first portion MD′ comprises a first energy storage unit 304a and a controller 300a comprising one or several processing units connected to the first energy storage unit 304a. The first energy storage unit 304a may be rechargeable by wireless transfer of energy. In some embodiments, the first energy storage unit 304a may be non-rechargeable. Upon reaching the lifetime end of such first energy storage, a replacement first portion comprising a new first energy storage unit may simply be swapped in place for the first portion having the depleted first energy storage unit. The second portion MD″ may further comprise a controller 300b comprising one or several processing units.

As can be seen in FIG. 30, the first, second, third and fourth planes P1, P2, P3 and P4, are parallel to each other. Furthermore, in the illustrated embodiment, the third cross-sectional area A3 is smaller than the first, second and fourth cross-sectional areas A1, A2 and A4 such that the first portion MD′, second portion MD″ and connecting portion MD-2 are prevented from traveling through the hole in the tissue portion 610 in a direction perpendicular to the first, second and third planes P1, P2 and P3. Hereby, the second portion MD″ and the connecting portion MD-2 can be held in position by the tissue portion 610 of the patient even if the first portion MD′ is disconnected from the connecting portion MD-2.

It is to be understood that the illustrated planes P1, P2, P3 and P4 are merely an example of how such planes may intersect the medical device MD. Other arrangements of planes are possible, as long as the conditions above are fulfilled, i.e. that the portions have cross-sectional areas, wherein the third cross-sectional area in the third plane P3 is smaller than the first, second and fourth cross-sectional areas, and that the planes P1, P2, P3 and P4 are parallel to each other.

The connecting portion MD-2 illustrated in FIG. 30 may be defined as a connecting portion MD-2 comprising a flange 626. The flange 626 thus comprises the fourth cross-sectional area A4 such that the flange 626 is prevented from traveling through the hole in the tissue portion 610 in a direction perpendicular to the first, second and third planes P1, P2 and P3. The flange 626 may protrude in a direction parallel to the first, second, third and fourth planes P1, P2, P3 and P4. This direction is perpendicular to a central extension C1 of the connecting portion MD-2.

The connecting portion MD-2 is not restricted to flanges, however. Other protruding elements may additionally or alternatively be incorporated into the connecting portion MD-2. As such, the connecting portion MD-2 may comprise at least one protruding element comprising the fourth cross-sectional area A4 such that the at least one protruding element is prevented from traveling through the hole in the tissue portion 610 such that the second portion MD″ and the connecting portion MD-2 can be held in position by the tissue portion 610 of the patient even if the first portion MD′ is disconnected from the connecting portion MD-2. The at least one protruding element may protrude in a direction parallel to the first, second, third and fourth planes P1, P2, P3 and P4. This direction is perpendicular to a central extension C1 of the connecting portion MD-2. As such, the at least one protruding element will also comprise the third surface 624 configured to engage the first tissue surface 616 of the first side 612 of the tissue portion 610.

The connecting portion MD-2 may comprise a hollow portion 628. The hollow portion 628 may provide a passage between the first and second portions MD′, MD″. In particular, the hollow portion 628 may house a conduit for transferring fluid from the first portion MD′ to the second portion MD″. The hollow portion 628 may also comprise or house one or several connections or electrical leads for transferring energy and/or communication signals between the first portion MD′ and the second portion MD″.

It is important to note that although the implantable energized medical device is disclosed herein as having a third cross-sectional area being smaller than a first cross-sectional area, this feature is not essential. The third cross-sectional area may be equal to or larger than the first cross-sectional area.

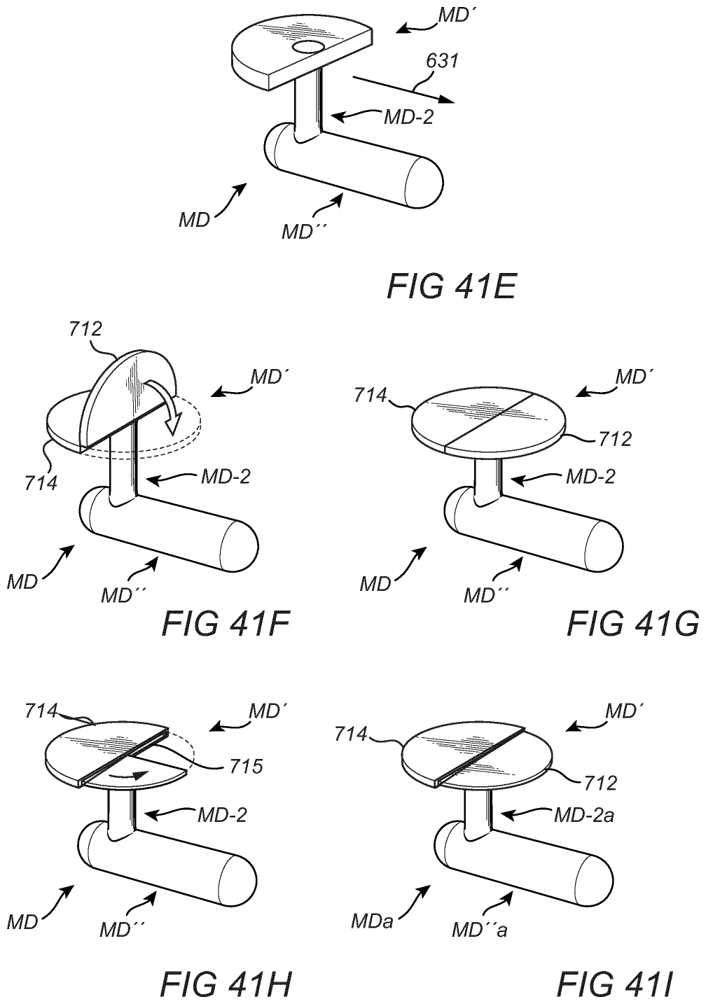

Some relative dimensions of the medical device MD will now be described with reference to FIGS. 30 and 31A to 31D. However, it is to be understood that these dimensions may also apply to other embodiments of the medical device MD. The at least one protruding element 626 may have a height HF in a direction perpendicular to the fourth plane P4 being less than a height H1 of the first portion MD′ in said direction. The height HF may alternatively be less than half of said height H1 of the first portion MD′ in said direction, less than a quarter of said height H1 of the first portion MD′ in said direction, or less than a tenth of said height H1 of the first portion MD′ in said direction.

The height H1 of the first portion MD′ in a direction perpendicular to the first plane P1 may be less than a height H2 of the second portion MD″ in said direction, such as less than half of said height H2 of the second portion MD″ in said direction, less than a quarter of said height H2 of the second portion MD″ in said direction, or less than a tenth of said height H2 of the second portion MD″ in said direction.

The at least one protruding element 626 may have a diameter DF in the fourth plane P4 being one of: less than a diameter D1 of the first portion MD′ in the first plane P1, equal to a diameter D1 of the first portion MD′ in the first plane P1, and larger than a diameter D1 of the first portion MD′ in the first plane P1. Similarly, the cross-sectional area of the at least one protruding element 626 in the fourth plane P4 may be less, equal to, or larger than a cross-sectional area of the first portion in the first plane P1.

The at least one protruding element 626 may have a height HF in a direction perpendicular to the fourth plane P4 being less than a height HC of the connecting portion MD-2 in said direction. Here, the height HC of the connecting portion MD-2 is defined as the height excluding the at least one protruding element, which forms part of the connecting portion MD-2. The height HF may alternatively be less than half of said height HC of the connecting portion MD-2 in said direction, less than a quarter of said height HC of the connecting portion MD-2 in said direction, or less than a tenth of said height HC of the connecting portion MD-2 in said direction.

As shown in FIG. 31D, the first portion MD′ may have a first cross-sectional area A1 being equal to or smaller than the third cross-sectional area A3 of the connecting portion MD-2. In particular, the first portion MD′ does not necessarily need to provide a cross-sectional area being larger than the third cross-sectional area of connecting portion MD-2, intended to pass through a hole in the tissue, if the connecting portion MD-2 provides an additional cross-sectional area being larger than the third cross-sectional area of the connecting portion MD-2. The first portion MD′ as illustrated in FIG. 31D may comprise the components discussed elsewhere in the present disclosure, although not shown, such as an energy storage unit, receiver, transmitter, etc.

Wireless energy receivers and/or communication receivers and/or transmitters in the first portion MD′ may be configured to receive energy from and/or communicate wirelessly with an external device outside the body using electromagnetic waves at a frequency below 100 kHz, or more specifically below 40 kHz, or more specifically below 20 kHz. The wireless energy receivers and/or communication receivers and/or transmitters in the first portion MD′ may thus be configured to communicate with the external device using “Very Low Frequency” communication (VLF). VLF signals have the ability to penetrate a titanium housing of the implantable energized medical device, such that the electronics of the implantable medical device can be completely encapsulated in a titanium housing. In addition, or alternatively, communication and energy transfer between the first portion MD′ and second portion MD″ may be made using VLF signals. In such embodiments, receivers and transmitters (for energy and/or communication) of the first portion MD′ and second portion MD″ are configured accordingly.

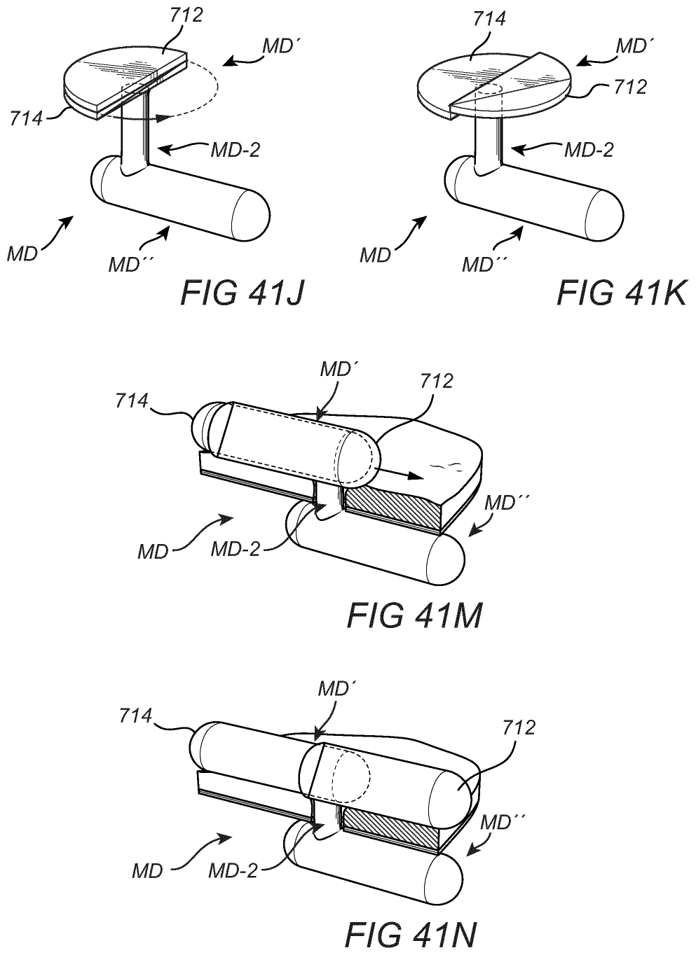

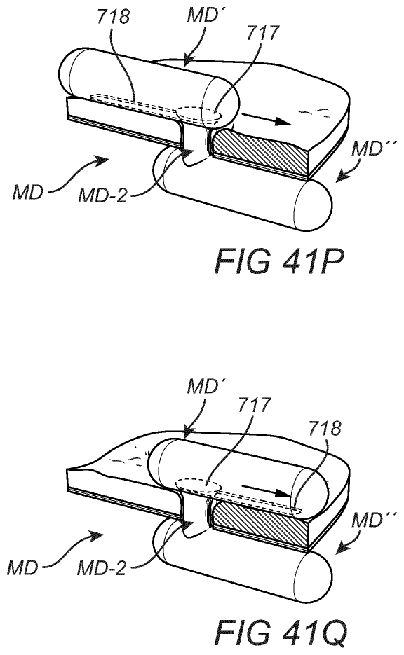

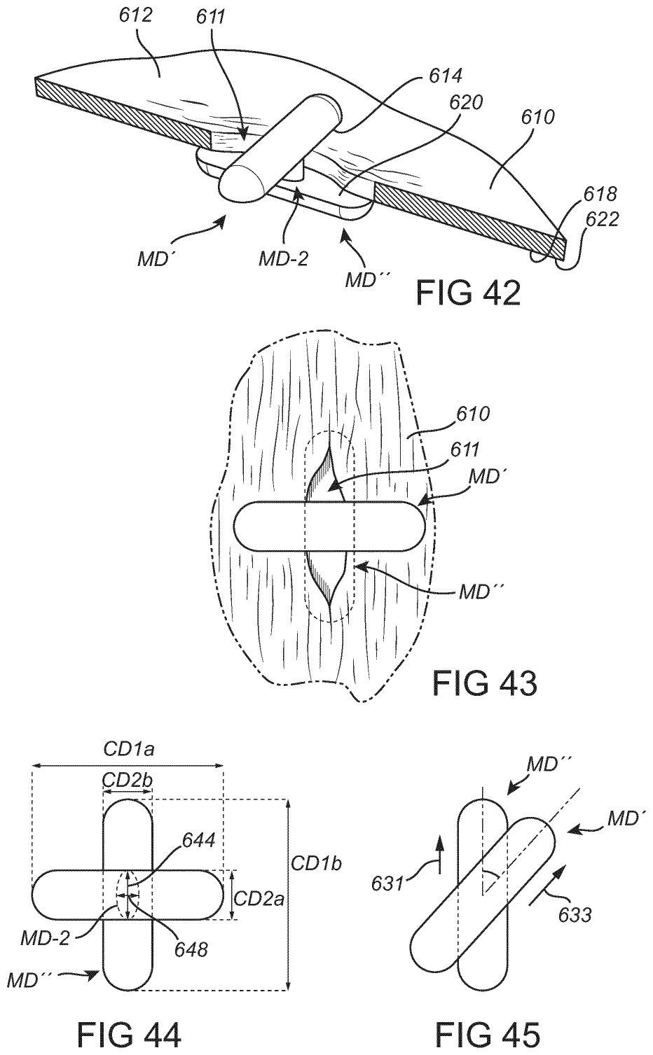

As shown in FIGS. 32A to 32B, the at least one protruding element 626 may have an annular shape, such as a disk shape. However, elliptical, elongated and/or other polyhedral or irregular shapes are also possible. In the illustrated embodiment, the at least one protruding element 626 extends a full revolution around the center axis of the connecting portion MD-2. However, other arrangements are possible, wherein the at least one protruding element 626 constitutes a partial circle sector. In the case of a plurality of protruding elements, such plurality of protruding elements may constitute several partial circle sectors.

As shown in FIGS. 30A to 30B, 31A to 31B, the connecting portion MD-2 may comprise at least two protruding elements 626, 627. For example, the connecting portion MD-2 may comprise at least three, four, five, six, seven, eight, nine, ten protruding elements, and so on. In such embodiments, the at least two protruding elements 626, 627 may together comprise the fourth cross-sectional area, thus providing a necessary cross-sectional area to prevent the first portion MD′ and second portion MD″ from traveling through the hole in the tissue portion 610.

The at least two protruding elements 626, 627 may be symmetrically arranged about the central axis of the connecting portion MD-2, as shown in FIGS. 30A to 30B, or asymmetrically arranged about the central axis of the connecting portion MD-2, as shown in FIGS. 31A to 31B. In particular, the at least two protruding elements 626, 627 may be asymmetrically arranged so as to be located towards one side of the connecting portion MD-2, as shown in FIGS. 31A to 31B. The arrangement of protruding element(s) may allow the medical device MD, and in particular the connecting portion MD-2, to be placed in areas of the patient where space is limited in one or more directions.

### Pop Rivet Kit

Although one type or embodiment of the implantable energized medical device MD, which may be referred to as a remote unit in other parts of the present disclosure, may fit most patients, it may be necessary to provide a selection of implantable energized medical devices MD or portions MD′, MD″ to be assembled into implantable energized medical devices MD. For example, some patients may require different lengths, shapes, sizes, widths or heights depending on individual anatomy. Furthermore, some parts or portions of the implantable energized medical device MD may be common among several different types or embodiments of implantable energized medical devices, while other parts or portions may be replaceable or interchangeable. Such parts or portions may include energy storage devices, communication devices, fluid connections, mechanical connections, electrical connections, and so on.

To provide flexibility and increase user-friendliness, a kit of parts may be provided. The kit preferably comprises a group of one or more first portions, a group of one or more second portions, and a group of one or more connecting portions, the first portions, second portions and connecting portions being embodied as described throughout the present disclosure. At least one of the groups comprises at least two different types of said respective portions. By the term “type”, it is hereby meant a variety, class or embodiment of said respective portion.

In some embodiments of the kit, the group of one or more first portions, the group of one or more second portions, and the group of one or more connecting portions, comprise separate parts which may be assembled into a complete implantable energized medical device. The implantable energized medical device MD may thus be said to be modular, in that the first portion MD′, the second portion MD″, and/or the connecting portion MD-2 may be interchanged for another type of the respective portion.

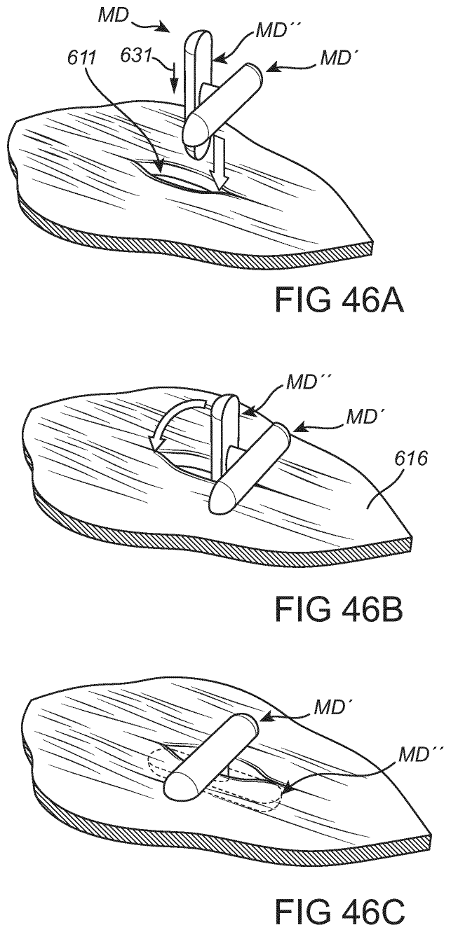

With reference to FIG. 35, the kit for assembling the implantable energized medical device MD comprises a group 650 of one or more first portions MD″, in the illustrated example a group of one first portion MD′, a group 652 of one or more connecting portions MD-2, in the illustrated example a group of three connecting portions MD-2, and a group 654 of one or more second portions MD″, in the illustrated example a group of two second portions MD″. For simplicity, all types and combinations of first portions MD′, second portions MD″ and connecting portions MD-2 will not be illustrated or described in detail.

Accordingly, the group 652 of one or more connecting portions MD-2 comprises three different types of connecting portions MD-2. Here, the different types of connecting portions MD-2 comprise connecting portions MD-2a, MD-2b, MD-2c having different heights. Furthermore, the group 654 of one or more second portions MD″ comprises two different types of second portions MD″.

Here, the different types of second portions MD″ comprise a second portion MD″ a being configured to eccentrically connect to a connecting portion, having a first end and a second end as described in other parts of the present disclosure, wherein the second end of the second portion MD″a comprises or is configured for at least one connection for connecting to an implant being located in a caudal direction from a location of the implantable energized medical device in the patient, when the medical device MD is assembled. In the illustrated figure, the at least one connection is visualized as a lead or wire. However, other embodiments are possible, including the second end comprising a port, connector or other type of connective element for transmission of power, fluid, and/or signals.

Furthermore, the different types of second portions MD″ comprise a second portion MD″b being configured to eccentrically connect to a connecting portion, having a first end and a second end as described in other parts of the present disclosure, wherein the first end of the second portion MD″b comprises or is configured for at least one connection for connecting to an implant being located in a cranial direction from a location of the implantable energized medical device in the patient, when the medical device MD is assembled. In the illustrated figure, the at least one connection is visualized as a lead or wire. However, other embodiments are possible, including the first end comprising a port, connector or other type of connective element for transmission of power, fluid, and/or signals.

Thus, the implantable energized medical device MD may be modular, and different types of medical devices MD can be achieved by selecting and combining a first portion MD′, a connecting portion MD-2, and a second portion MD″, from each of the groups 652, 654, 656.

In the illustrated example, a first implantable energized medical device MDa is achieved by a selection of the first portion MD′, the connecting portion MD-2a, and the second portion MD″a. Such device MDa may be particularly advantageous in that the connecting portion MD-2a may be able to extend through a thick layer of tissue to connect the first portion MD′ and the second portion MD″a. Another implantable energized medical device MDb is achieved by a selection of the first portion MD′, the connecting portion MD-2S, and the second portion MD″b. Such device may be particularly advantageous in that the connecting portion MD-2c has a smaller footprint than the connecting portion MD-2a, i.e. occupying less space in the patient. Owing to the modular property of the medical devices MDa and MDb, a practician or surgeon may select a suitable connecting portion as needed upon having assessed the anatomy of a patient. Furthermore, since devices MDa and MDb share a common type of first portions MD′, it will not be necessary for a practitioner or surgeon to maintain a stock of different first portions MD′ (or a stock of complete, assembled medical devices MD) merely for the sake of achieving a medical device MD having different connections located in the first end or second end of the second portion MD″ respectively, as in the case of second portions MD″a. MD″b.

The example illustrated in FIG. 35 is merely exemplifying to display the idea of a modular implantable energized medical device MD. The group 650 of one or more first portions MD′ may comprise a variety of different features, such as first portions with or without a first energy storage unit, with or without a first wireless energy receiver unit for receiving energy transmitted wirelessly by an external wireless energy transmitter, with or without an internal wireless energy transmitter, and/or other features as described throughout the present disclosure. Other features include different height, width, or length of the first portion. It is to be understood that first portions MD′ having one or more such features may be combined with a particular shape or dimension to achieve a variety of first portions. The same applies to connecting portions MD-2 and second portions MD″.

### Pop Rivet Internal Wireless

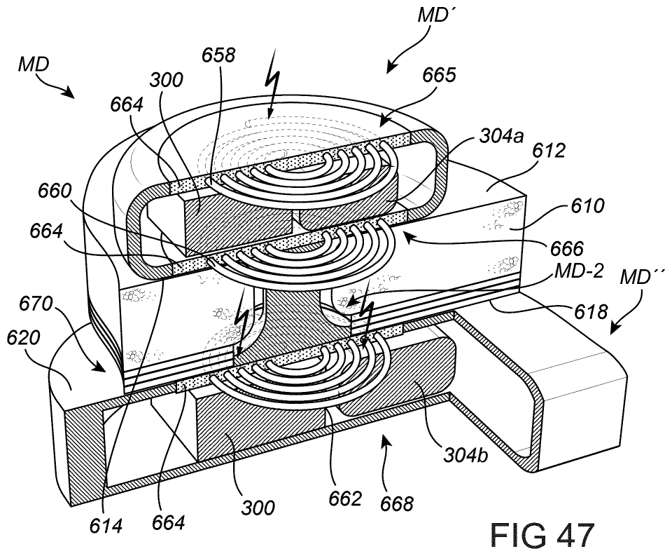

With reference to FIG. 36, an embodiment of an implantable energized medical device MD, which may be referred to as a remote unit in other parts of the present disclosure, will be described. The medical device MD is configured to be held in position by a tissue portion 610 of a patient. The medical device MD comprises a first portion MD′ configured to be placed on a first side of the tissue portion 610, the first portion MD′ having a first cross-sectional area in a first plane P1 and comprising a first surface configured to face and/or engage a first tissue surface of the first side of the tissue portion 610. The medical device MD further comprises a second portion MD″ configured to be placed on a second side of the tissue portion 610, the second side opposing the first side, the second portion MD″ having a second cross-sectional area in a second plane and comprising a second surface configured to engage a second tissue surface of the second side of the tissue portion 610. The medical device MD further comprises a connecting portion MD-2 configured to be placed through a hole in the tissue portion 610 extending between the first and second sides of the tissue portion 610. Here, the connecting portion MD-2 has a third cross-sectional area in a third plane. The connecting portion MD-2 is configured to connect the first portion MD′ to the second portion MD″. Here, the first portion MD′ comprises a first wireless energy receiver 308a for receiving energy transmitted wirelessly by an external wireless energy transmitter, and an internal wireless energy transmitter 308a configured to transmit energy wirelessly to the second portion. Furthermore, here the second portion comprises a second wireless energy receiver 308b configured to receive energy transmitted wirelessly by the internal wireless energy transmitter 308a.

Although receivers and transmitters may be discussed and illustrated separately in the present disclosure, it is to be understood that the receivers and/or transmitters may be comprised in a transceiver. Furthermore, the receivers and/or transmitters in the first portion MD′ and second portion MD″, respectively, may form part of a single receiving or transmitting unit configured for receiving or transmitting energy and/or communication signals, including data. Furthermore, the internal wireless energy transmitter and/or a first wireless communication receiver/transmitter may be a separate unit 308c located in a lower portion of the first portion MD′ close to the connecting portion MD-2 and the second portion MD″. Such placement may provide that energy and/or communication signals transmitted by the unit 308c will not be attenuated by internal components of the first portion MD′ when being transmitted to the second portion MD″. Such internal components may include a first energy storage unit 304a.

The first portion MD′ here comprises a first energy storage unit 304a connected to the first wireless energy receiver 308a. The second portion comprises a second energy storage unit 304b connected to the second wireless energy receiver 308b. Such an energy storage unit may be a solid-state battery, such as a thionyl chloride battery.

In some embodiments, the first wireless energy receiver 308a is configured to receive energy transmitted wirelessly by the external wireless energy transmitter and store the received energy in the first energy storage unit 304a. Furthermore, the internal wireless energy transmitter 308a is configured to wirelessly transmit energy stored in the first energy storage unit 304a to the second wireless energy receiver 308b, and the second wireless energy receiver 308b is configured to receive energy transmitted wirelessly by the internal wireless energy transmitter 308a and to store the received energy in the second energy storage unit 305b.

The first energy storage unit 304a may be configured to store less energy than the second energy storage unit 304b, and/or configured to be charged faster than the second energy storage unit 304b. Hereby, charging of the first energy storage unit 304a may be relatively quick, whereas transfer of energy from the first energy storage unit 304a to the second energy storage unit 304b may be relatively slow. Thus, a user can quickly charge the first energy storage unit 304a, and will not-during such charging—be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b, via the first wireless energy transmitter 308a, 308c and the second wireless energy receiver 308b.

The first portion may comprise a first controller comprising at least one processing unit 306a. The second portion may comprise a second controller comprising at least one processing unit 306b. At least one of the first and second processing unit 306a. 306b may be connected to a wireless transceiver 308a, 308b, 308c for communicating wirelessly with an external device.

The first controller may be connected to a first wireless communication receiver 308a. 308c in the first portion MD′ for receiving wireless communication from an external device and/or from a wireless communication transmitter 308b in the second portion MD″. Furthermore, the first controller may be connected to a first wireless communication transmitter 308a, 308c in the first portion MD′ for transmitting wireless communication to a second wireless communication receiver 308b in the second portion MD″. The second controller may be connected to the second wireless communication receiver 308b for receiving wireless communication from the first portion MD′. The second controller may further be connected to a second wireless communication transmitter 308b for transmitting wireless communication to the first portion MD″.

In some embodiments, the first wireless energy receiver 308a comprises a first coil, and the wireless energy transmitter 308a, 308c comprises a second coil.

Pop rivet shoe

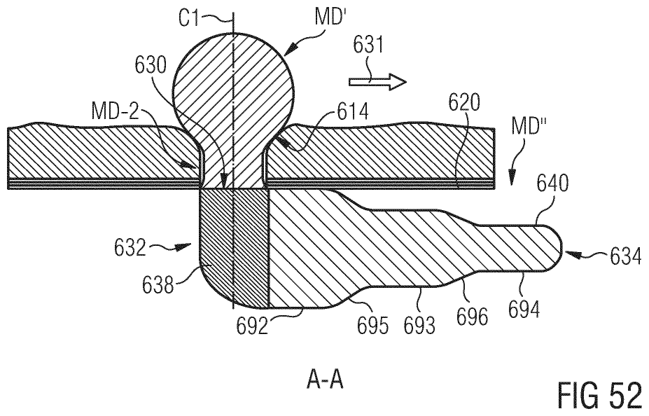

With reference to FIGS. 37, 40A and 40B, an embodiment of an implantable energized medical device MD, which may be referred to as a remote unit in other parts of the present disclosure, will be described. The medical device MD is configured to be held in position by a tissue portion 610 of a patient. The medical device MD comprises a first portion MD′ configured to be placed on a first side 612 of the tissue portion 610, the first portion MD′ having a first cross-sectional area A1 in a first plane P1 and comprising a first surface 614 configured to face and/or engage a first tissue surface 616 of the first side 612 of the tissue portion 610. The medical device MD further comprises a second portion MD″ configured to be placed on a second side 618 of the tissue portion 610, the second side 618 opposing the first side 612, the second portion MD″ having a second cross-sectional area A2 in a second plane P2 and comprising a second surface 620 configured to engage a second tissue surface 622 on the second side 618 of the tissue portion 610. The medical device MD further comprises a connecting portion MD-2 configured to be placed through a hole in the tissue portion 610 extending between the first and second sides 612, 618 of the tissue portion 610. Here, the connecting portion MD-2 has a third cross-sectional area A3 in a third plane P3. The connecting portion MD-2 is configured to connect the first portion MD′ to the second portion MD″. In the illustrated embodiment, a connecting interface 630 between the connecting portion MD-2 and the second portion MD″ is eccentric with respect to the second portion MD″.

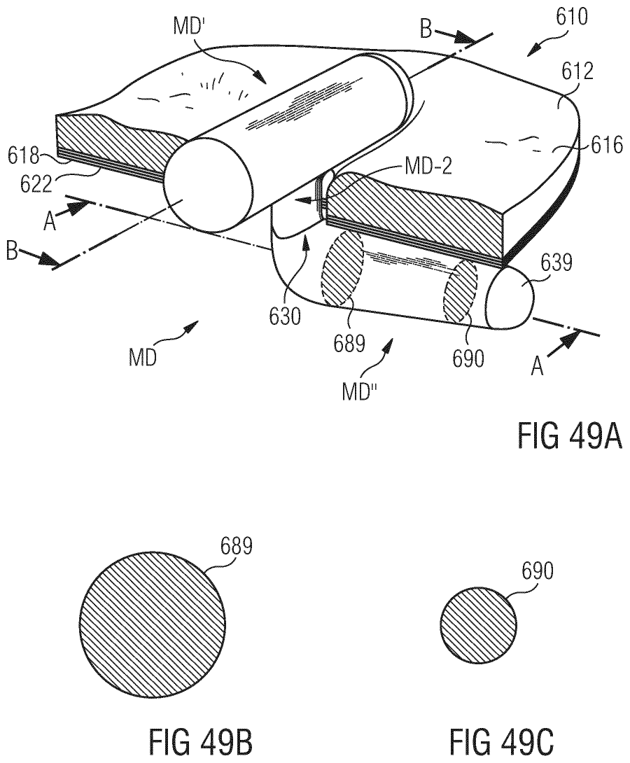

The first portion MD′ has an elongated shape in the illustrated embodiment of FIG. 1. Similarly, the second portion MD″ has an elongated shape. However, the first portion MD′ and/or second portion MD″ may assume other shapes, such as a flat disk, e.g., having a width and length being larger than the height, a sphere, an ellipsoid, or any other polyhedral or irregular shape, some of these being exemplified in FIGS. 37 to 38.

As illustrated in FIGS. 40A and 40B, the connecting interface 630 between the connecting portion MD-2 and the second portion MD″ may be eccentric, with respect to the second portion MD″ in a first direction 631, but not in a second direction 633 being perpendicular to the first direction. The first direction 631 is here parallel to the line A-A, to the second plane P2, and to a length of the second portion MD″. The second direction 633 is here parallel to the line B-B, to the second plane P2, and to a width of the second portion MD″. It is also possible that the connecting interface between the connecting portion MD-2 and the second portion MD″ is eccentric, with respect to the second portion MD″, in the first direction 631 as well as in the second direction 633 being perpendicular to the first direction 631.

Similarly, a connecting interface between the connecting portion MD-2 and the first portion MD′ may be eccentric with respect to the first portion MD′ in the first direction 631 and/or in the second direction 633.

The first portion MD′, connecting portion MD-2 and second portion MD″ may structurally form one integral unit. It is, however, also possible that the first portion MD′ and the connecting portion MD-2 structurally form one integral unit while the second portion MD″ forms a separate unit, or that the second portion MD″ and the connecting portion MD-2 structurally form one integral unit while the first portion MD′ forms a separate unit.

Additionally, or alternatively, the second portion MD″ may comprise a removable and/or interchangeable portion 639. In some embodiments, the removable portion 639 may form part of a distal region. A removable portion may also form part of a proximal region. Thus, the second portion MD″ may comprise at least two removable portions, each being arranged at a respective end of the second portion MD″. The removable portion 639 may house, hold or comprise one or several functional parts of the medical device MD, such as gears, motors, connections, reservoirs, and the like as described in other parts of the present disclosure. An embodiment having such a removable portion 639 will be able to be modified as necessary to circumstances of a particular patient.

In the case of the first portion MD″, connecting portion MD-2 and second portion MD″ structurally forming one integral unit, the eccentric connecting interface between the connecting portion MD-2 and the second portion MD″, with respect to the second portion MD″, will provide that the medical device MD will be able to be inserted into the hole in the tissue portion. The medical device MD may for example be inserted into the hole at an angle, similar to how a foot is inserted into a shoe, to allow most or all of the second portion MD″ to pass through the hole, before it is angled, rotated and/or pivoted to allow any remaining portion of the second portion MD″ to pass through the hole and allow the medical device MD to assume its intended position.

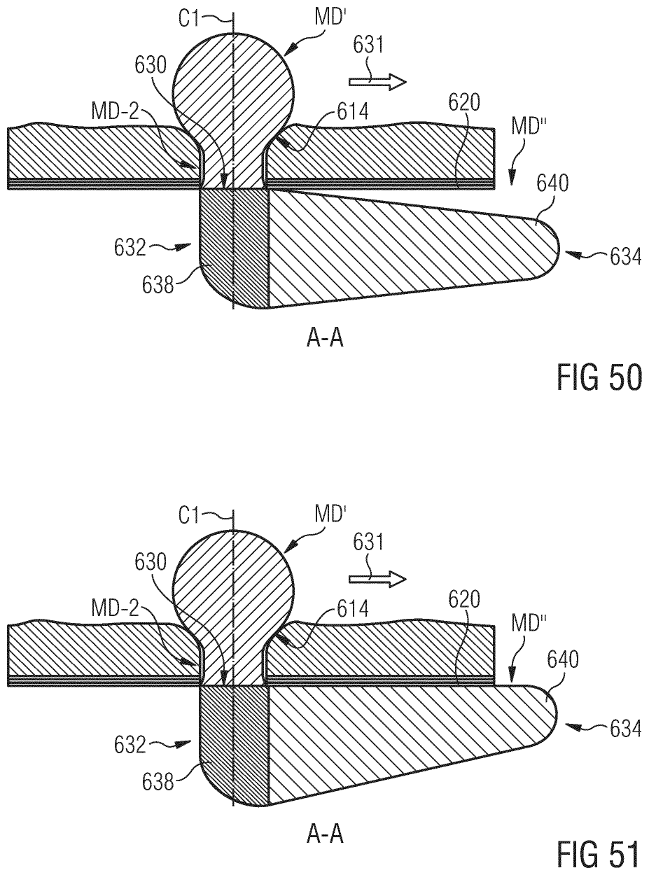

As illustrated in FIGS. 37, 38 and 39, the first portion MD′ may assume a variety of shapes, such as an oblong shape, a flat disk shape, a spherical shape, or any other polyhedral or irregular shape. Similarly, the second portion MD″ may assume a variety of shapes, such as an oblong shape, a flat disk shape, a spherical shape, or any other polyhedral or irregular shape. The proposed shapes of the first and second portions MD′, MD″ may be mixed and combined to form embodiments not exemplified in the illustrated embodiments. For example, one or both of the first and second portions MD′, MD″ may have a flat oblong shape. In this context, the term “flat” is related to the height of the first or second portion MD′, MD″, i.e. in a direction parallel to a central extension C1 of the connecting portion MD-2. The term “oblong” is related to a length of the first or second portion MD″, MD″.

With reference to FIGS. 40A to 40B, the second portion MD″ has a first end 632 and a second end 634 opposing the first end 632. The length of the second portion MD″ is defined as the length between the first end 632 and the second end 634. The length of the second portion MD″ is furthermore extending in a direction being different from the central extension C1 of the connecting portion MD-2. The first end 632 and second end 634 are separated in a direction parallel to the second plane P2. Similarly, the first portion MD′ has a length between a first and a second end, the length extending in a direction being different from the central extension C1 of the connecting portion MD-2.

The second portion MD″ may be curved along its length. For example, one or both ends of the second portion MD″ may point in a direction being substantially different from the second plane P2, i.e. curving away from or towards the tissue portion when implanted. In some embodiments, the second portion MD″ curves within the second plane P2, exclusively or in combination with curving in other planes. The second portion MD″ may also be curved in more than one direction, i.e. along its length and along its width, the width extending in a direction perpendicular to the length.

The first and second ends 632, 634 of the second portion MD″ may respectively comprise an elliptical point. For example, the first and second ends 632, 634 may comprise a hemispherical end cap respectively. It is to be understood that also the first and second ends of the first portion MD′ may have such features.

The second portion MD″ may have at least one circular cross-section along the length between the first end 632 and second end 634, as illustrated in FIG. 37. It is, however, possible for the second portion MD″ to have at least one oval cross-section or at least one elliptical cross-section along the length between the first end 632 and the second end 634. Such cross-sectional shapes may also exist between ends in a width direction of the second portion MD″. Similarly, such cross-sectional shapes may also exist between ends in a length and/or width direction in the first portion MD′.

In the following paragraphs, some features and properties of the second portion MD″ will be described. It is, however, to be understood that these features and properties may also apply to the first portion MD′.

The second portion MD″ has a proximal region 636, an intermediate region 638, and a distal region 640. The proximal region 636 extends from the first end 632 to an interface between the connecting portion MD-2 and the second portion MD″, the intermediate region 638 is defined by the connecting interface 630 between the connecting portion MD-2 and the second portion MD″, and the distal region 640 extends from the connecting interface 630 between the connecting portion MD-2 and the second portion MD″ to the second end 634. The proximal region 636 is shorter than the distal region 640 with respect to the length of the second portion, i.e. with respect to the length direction 631. Thus, a heel (the proximal region) and a toe (the distal region) are present in the second portion MD″.

The second surface 620, configured to engage with the second tissue surface 622 of the second side 618 of the tissue portion 610, is part of the proximal region 636 and the distal region 640. If a length of the second portion MD″ is defined as x, and the width of the second portion MD″ is defined as y along respective length and width directions 631, 633 being perpendicular to each other and substantially parallel to the second plane P2, the connecting interface between the connecting portion MD-2 and the second portion MD″ is contained within a region extending from x>0 to x<x/2 and/or y>0 to y<y/2, x and y and 0 being respective end points of the second portion MD″ along said length and width directions. In other words, the connecting interface between the connecting portion MD-2 and the second portion MD″ is eccentric in at least one direction with respect to the second portion MD″ such that a heel and a toe are formed in the second portion MD″.

The first surface 614 configured to face and/or engage the first tissue surface 616 of the first side 612 of the tissue portion 610 may be substantially flat. In other words, the first portion MD′ may comprise a substantially flat side facing towards the tissue portion 610. Furthermore, an opposing surface of the first portion MD′, facing away from the tissue portion 610, may be substantially flat. Similarly, the second surface 620 configured to engage the second tissue surface 622 of the second side 618 of the tissue portion 610 may be substantially flat. In other words, the second portion MD″ may comprise a substantially flat side facing towards the tissue portion 610. Furthermore, an opposing surface of the second portion MD″, facing away from the tissue portion 610, may be substantially flat.

The second portion MD″ may be tapered from the first end 632 to the second end 634, thus giving the second portion MD″ different heights and/or widths along the length of the second portion MD″. The second portion may also be tapered from each of the first end 632 and second end 634 towards the intermediate region 638 of the second portion MD″.

Some dimensions of the first portion MD′, the second portion MD″ and the connecting portion MD-2 will now be disclosed. Any of the following disclosures of numerical intervals may include or exclude the end points of said intervals.

The first portion MD′ may have a maximum dimension in the range of 10 to 60 mm, such as in the range of 10 to 40 mm, such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm. By the term “maximum dimension” it is hereby meant the largest dimension in any direction.

The first portion MD′ may have a diameter in the range of 10 to 60 mm, such as in the range of 10 to 40 mm, such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm.

The connecting portion MD-2 may have a maximum dimension in the third plane P3 in the range of 2 to 20 mm, such as in the range of 2 to 15 mm, such as in the range of 2 to 10 mm, such as in the range of 5 to 10 mm, such as in the range of 8 to 20 mm, such as in the range of 8 to 15 mm, such as in the range of 8 to 10 mm.

The second portion MD″ may have a maximum dimension in the range of 30 to 90 mm, such as in the range of 30 to 70 mm, such as in the range of 30 to 60 mm, such as in the range of 30 to 40 mm, such as in the range of 35 to 90 mm, such as in the range of 35 to 70 mm, such as in the range of 35 to 60 mm, such as in the range of 35 to 40 mm.

The first portion has a first height H1, and the second portion has a second height H2, both heights being in a direction perpendicular to the first and second planes P1, P2. The first height may be smaller than the second height. However, in the embodiments illustrated in FIGS. 40A to 40B, the first height H1 is substantially equal to the second height H2. Other height ratios are possible, for example the first height H1 may be less than ⅔ of the second height H2, such as less than ½ of the second height H2, such as less than ⅓ of the second height H2, such as less than ¼ of the second height H2, such as less than ⅕ of the second height H2, such as less than 1/10 of the second height H2.

As illustrated in FIGS. 40A to 40B, the proximal region 636 has a length 642 being smaller than a length 646 of the distal region 640. The intermediate region 638 has a length 644, and a width 648. In some embodiments, the length 644 of the intermediate region 638 is greater than the width 648. In other words, the connecting interface between the connecting portion MD-2 and the second portion MD″ may be elongated, having a longer dimension (in the exemplified case, the length) and a shorter dimension (in the exemplified case, the width). It is also possible that the length 644 of the intermediate region 638 is shorter than the width 648 of the intermediate region 638.

The length 646 of the distal region 640 is preferably greater than the length 644 of the intermediate region 638, however, an equally long distal region 640 and intermediate region 638 or a shorter distal region 640 than the intermediate region 638 are also possible. The length 642 of the proximal region 636 may be smaller than, equal to, or greater than the length 644 of the intermediate region 638.

The length 644 of the intermediate region 638 is preferably less than half of the length of the second portion MD″, i.e. less than half of the combined length of the proximal region 636, the intermediate region 638, and the distal region 630. In some embodiments, the length 644 of the intermediate region 638 is less than a third of the length of the second portion MD″, such as less than a fourth, less than a fifth, or less than a tenth of the length of the second portion MD″.

The connecting portion may have one of an oval cross-section, an elongated cross-section, and a circular cross-section, in a plane parallel to the third plane P3. In particular, the connecting portion may have several different cross-sectional shapes along its length in the central extension C₁.

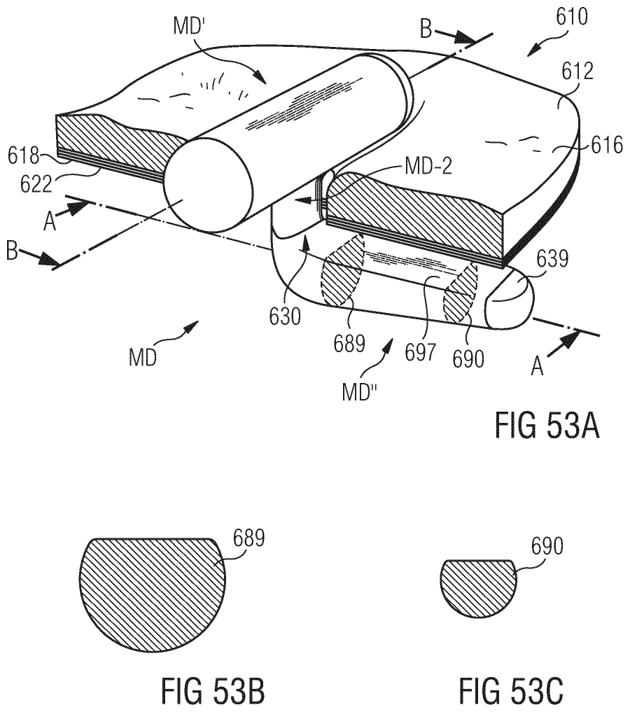

In some embodiments the distal region 640 is configured to be directed downwards in a standing patient, i.e. in a caudal direction when the medical device MD is implanted. As illustrated in FIGS. 41A to 41D, different orientations of the second portion MD″ relative to the first portion MD′ are possible. In some embodiments, a connection between either the first portion MD′ and the connecting portion MD-2 or between the second portion MD″ and the connecting portion MD-2 may allow for a plurality of different connecting orientations. For example, a connection mechanism between the first portion MD′ and the connecting portion MD-2 (or between the second portion MD″ and the connecting portion MD-2) may posses a 90-degrees rotational symmetry to allow the second portion MD′ to be set in four different positions with respect to the first portion MD, each differing from the other by 90 degrees. Other degrees of rotational symmetry are, of course, possible, such as 30 degrees, 45 degrees, 60 degrees, 120 degrees, 180 degrees and so on. In other embodiments there are no connective mechanisms between any of the first portion MD′, the connecting portion MD-2, and the second portion MD″ (i.e. the portions are made as one integral unit), and in such cases different variants of the medical device MD can be achieved during manufacturing. In other embodiments, the connective mechanism between the first portion MD′ and the connecting portion MD-2 (or between the second portion MD″ and the connecting portion MD-2) is non-reversible, i.e. the first portion MD″ and the second portion MD″ may initially be handled as separate parts, but the orientation of the second portion MD″ relative to the first portion MD′ cannot be changed once it has been selected and the parts have been connected via the connecting portion MD-2.

The different orientations of the second portion MD″ relative to the first portion MD′ may be defined as the length direction of the second portion MD″ having a relation or angle with respect to a length direction of the first portion MD′. Such angle may be 15, 30, 45, 60, 75 90, 105, 120, 135, 150, 165, 180, 195, 210, 225, 240, 255, 270, 285, 300, 315, 330, 345 or 360 degrees. In particular, the angle between the first portion MD′ and the second portion MD″ may be defined as an angle in the planes P1 and P2, or as an angle in a plane parallel to the tissue portion 610, when the medical device MD is implanted. In the embodiment illustrated in FIGS. 41A to 41D, the length direction of the second portion MD″ is angled by 0, 90, 180, and 270 degrees with respect to the length direction of the first portion MD″.

The second end 634 of the second portion MD″ may comprise one or several connections for connecting to an implant being located in a caudal direction from a location of the implantable energized medical device MD in the patient. Hereby, when the medical device MD is implanted in a patient, preferably with the distal region 640 and second end 634 pointing downwards in a standing patient, the connections will be closer to the implant as the second end 634 will be pointing in the caudal direction whereas the first end 632 will be pointing in the cranial direction. It is also possible that the second end 634 of the second portion MD″ is configured for connecting to an implant, i.e. the second end 634 may comprise a port, connector or other type of connective element for transmission of power, fluid and/or signals.

Likewise, the first end 632 of the second portion MD″ may comprise one or several connections for connecting to an implant which is located in a cranial direction from a location of the implantable energized medical device MD in the patient. Hereby, when the medical device MD is implanted in a patient, preferably with the distal region 640 and second end 634 pointing downwards in a standing patient, the connections will be closer to the implant as the first end 632 will be pointing in the cranial direction whereas the second end 634 will be pointing in the caudal direction. It is also possible that the first end 632 of the second portion MD″ is configured for connecting to an implant. i.e. the first end 632 may comprise a port, connector or other type of connective element for transmission of power, fluid and/or signals.

Referring now to FIGS. 41E-K, 41M, 41N, 41P and 41Q. The following will discuss some features of the first portion MD′, and in some cases additionally or alternatively of the connecting portion MD-2, which enable the first portion MD′ to increase its cross-sectional area in the first plane (i.e. to increase an area of the first surface configured to face the first tissue surface), and/or which enable the first portion MD′ to be rotated, translated, or otherwise moved in relation to the connecting portion MD-2. In some embodiments, the first portion MD′ will be configured to extend further away from the connecting portion MD-2 in or within the first plane. It is to be understood that these features can be combined with other features of the implantable energized medical device. In particular, the specific shape of the first portion, connecting portion and/or second portion in the illustrated embodiments are merely exemplary. Other shapes are possible, as discussed in the present disclosure. Accordingly, the elongated second portion MD″ does not necessarily need to be elongated as shown for example in FIG. 41E, and furthermore, the first portion MD′ does not necessarily need to have a semicircular shape.

With reference to FIG. 41E, an implantable energized medical device MD is shown, wherein the first portion MD′ is configured and shaped such that an edge 710 of the first portion MD′ is substantially aligned with the connecting portion MD-2 with regard to the first direction 631. In other words, no part of the first portion MD′ protrudes forward of the connecting portion MD-2 with regard to the first direction 631. Hereby, insertion of the implantable energized medical device MD may be facilitated, in particular when angled downwards, since the first portion MD′ will not abut the tissue until most or all of the second portion MD″ has been inserted through the hole in the tissue. Although the edge 710, as well as other edges of the first portion MD′, are hereby shown as having no radius, radiused edges are possible. Thus, the edge 710 may have a radius, and/or the first portion MD″, and/or the second portion MD″, and/or the connecting portion MD-2, may comprise radiused edges.

With reference to FIGS. 41F and 41G, a first portion MD′ is shown being configured to have its surface area increased. Here, the first cross-sectional area is increased, thereby increasing an area of the first surface configured to face (and in some embodiments also configured to contact) the first tissue surface. In the illustrated embodiment, the first portion MD′ comprises a first element 712 and a second element 714 being hingedly interconnected to allow the first element 712 to assume a first state (not shown) wherein the first element 712 is arranged on top of the second element 714, and a second state wherein the first element 712 is folded to be located adjacent or next to the second element 714. A similar configuration may be achieved by other means of interconnection between the first element 712 and second element 714, i.e. the configuration is not limited to a hinge-type connection. For example, the first element 712 and second element 714 may be constructed of a single piece of material being flexible enough to be able to fold over itself to assume the first and second state respectively.

Preferably, the first and second element 712. 714 are interconnected and formed such that a transition between the first and second element 712. 714 along the first direction 631 is flush.

Furthermore, while in the first state, the first portion MD′ may possess the same feature as discussed in conjunction with FIG. 41E, i.e. the first portion MD′ may be substantially aligned with the connecting portion MD-2.

With reference to FIGS. 41H and 41I, a first portion MD′ is shown being configured to have its surface area increased. Here, the first cross-sectional area is increased, thereby increasing an area of the first surface configured to face (and in some embodiments also configured to contact) the first tissue surface. In the illustrated embodiment, the first portion MD′ comprises a first element 712 and a second element 714. The second element 714 here comprises a slot 715 configured to partially or fully house the first element 712. The first element 712 is configured to rotate about an axis to assume a first state, wherein the first element 712 is partially or completely housed in within the slot 715, and a second state wherein the first element 712 protrudes from the slot 715 to increase the first cross-sectional area. The first element 712 may be configured to rotate 180 degrees about the axis. In the illustrated example, the first and second elements 712. 714 are shaped as semi-circles and form a shape conforming to a full circle in the second state. However, it is also possible that the first element 712 only rotate about the axis up to 90 degrees, thus forming a shape conforming to three quarters of a circle in the second state. Other shapes are also possible, e.g. polygons.

With reference to FIGS. 41J and 41K, a similar configuration as described with reference to FIGS. 41H and 41I is shown. However, here the second element 714 does not comprise a slot, and the first element is thus not housed in a slot. Instead, the first element 712 is arranged on top of the second element 714 (similar to the embodiment of FIGS. 41F and 41G). The first portion MD′ is here configured to have its surface area increased, in particular the first cross-sectional area is increased, thereby increasing an area of the first surface configured to face (and in some embodiments also configured to contact) the first tissue surface. The first element 712 is configured to rotate about an axis to assume a first state, wherein the first element 712 is partially or completely arranged on top of the second element 714. Here. “completely arranged on top of” means that the first element 712 is confined within the borders of the second element 714. By rotation of the first element 712 about the axis, the first element 712 can assume a second state wherein the first element 712 protrudes over an edge or border of the second element 714 to increase the first cross-sectional area. The first element 712 may be configured to rotate 180 degrees about the axis. However, it is also possible that the first element 712 only rotate about the axis up to 90 degrees. Other shapes of the first and second element 712. 714 are also possible, e.g. polygons.

With reference to FIGS. 41M and 41N, a first portion MD′ is shown being configured to have its surface area increased. Here, the first cross-sectional area is increased, thereby increasing an area of the first surface configured to face (and in some embodiments also configured to contact) the first tissue surface. In the illustrated embodiment, the first portion MD′ comprises a first element 712 and a second element 714. The first element 712 here comprises a slot configured to partially or completely house the second element 714. The first element 712 is configured to assume a first state, as shown in FIG. 41M, wherein the second element 714 is arranged partially or fully within the slot of the first element 712, and a second state, as shown in FIG. 41N, wherein the first element 712 has been moved in a first direction to cause the second element 714 to protrude from the slot of the first element 712, and to cause the first element 712 to extend further away from the connecting portion MD-2 in the first plane. As will be understood, other variations are possible, e.g. the second element 714 may comprise the slot, and the first element 712 may be partially or fully housed within such slot, and subsequently the first element 712 or the second element 714 may be moved to protrude from such slot.

With reference to FIGS. 41P and 41Q, a first portion MD′ is shown being configured to be moved in relation to the connecting portion MD-2. The expression “configured to be moved” may in this context be interpreted as the first portion MD′ being configured to assume at least two different positions with regard to the connecting portion MD-2 while still remaining in direct contact with the connecting portion. Here, the connecting portion MD-2 comprises a protruding element 717 and the first portion MD′ comprises a slot 718, wherein the protruding element 717 is configured to slide within the slot 718 along a predetermined path, e.g. in a first direction and a direction opposite said first direction. The protruding element 717 may be configured to be interlocked within the slot 718 such that the protruding element 717 can only be removed from the slot 718 in a preconfigured position. In other embodiments, the protruding element 717 may be permanently enclosed within the slot 718. By sliding the first portion MD′ in the first direction, an extension of the first portion MD′ in the first plane with respect to the connecting portion MD-2 will be able to be adjusted. Any position between the endpoints of the slot 718 may be able to be assumed by the first portion MD′. In particular, first portion MD′ and/or the connecting portion MD-2 may comprise a locking mechanism configured to secure a position of the first portion MD′ in relation to the connecting portion MD-2. Such locking mechanism may rely on flexible parts being biased towards each other to maintain the first portion MD′ and connecting portion MD-2 in a fixed position in relation to each other. Other possible locking mechanisms include the use of friction, snap-locking means, etc.

### Pop Rivet Cross

With reference to FIGS. 42 and 43, an embodiment of an implantable energized medical device MD, which may be referred to as a remote unit in other parts of the present disclosure, will be described. The medical device MD is configured to be held in position by a tissue portion 610 of a patient. The medical device MD comprises a first portion MD′ configured to be placed on a first side 612 of the tissue portion 610, the first portion MD′ having a first cross-sectional area in a first plane P1 and comprising a first surface 614 configured to face and/or engage a first tissue surface 616 on the first side 612 of the tissue portion 610. The medical device MD further comprises a second portion MD″ configured to be placed on a second side 618 of the tissue portion 610, the second side 618 opposing the first side 612, the second portion MD″ having a second cross-sectional area in a second plane and comprising a second surface 620 configured to engage a second tissue surface 622 on the second side 618 of the tissue portion 610. The medical device MD further comprises a connecting portion MD-2 configured to be placed through a hole in the tissue portion 610 extending between the first and second sides 612. 618 of the tissue portion 610. The connecting portion MD-2 here has a third cross-sectional area in a third plane. The connecting portion MD-2 is configured to connect the first portion MD′ to the second portion MD″.

With reference to FIG. 44, the first cross-sectional area has a first cross-sectional distance CD1a and a second cross-sectional distance CD2a, the first and second cross-sectional distances CDla. CD2a being perpendicular to each other and the first cross-sectional distance CD1a being longer than the second cross-sectional distance CD2a. Furthermore, the second cross-sectional area has a first cross-sectional distance CD1b and a second cross-sectional distance CD2b, the first and second cross-sectional distances CD2a. CD2b being perpendicular to each other and the first cross-sectional distance CD1b being longer than the second cross-sectional distance CD2b. The first cross-sectional distance CD1a of the first cross-sectional area and the first cross-sectional distance CD1b of the second cross-sectional area are rotationally displaced in relation to each other by an angle exceeding 45 degrees to facilitate insertion of the second portion MD″ through the hole in the tissue portion 610. In the embodiment illustrated in FIG. 44, the rotational displacement is 90 degrees.

The rotational displacement of the first portion MD′ and second portion MD″ forms a cross-like structure, being particularly advantageous in that insertion through the hole in the tissue portion 610 may be facilitated and, once positioned in the hole in the tissue portion 610, a secure position may be achieved. In particular, if the medical device MD is positioned such that the second portion MD″ has its first cross-sectional distance CD1b extending along a length extension of the hole 611 in the tissue portion 610, insertion of the second portion MD″ through the hole 611 may be facilitated. Furthermore, if the first portion MD′ is then displaced in relation to the second portion MD″ such that the first cross-sectional distance CD1a of the first portion MD′ is displaced in relation to a length extension of the hole 611, the first portion MD′ may be prevented from traveling through the hole 611 in the tissue portion. In these cases, it is particularly advantageous if the hole 611 in the tissue portion is oblong, ellipsoidal or at least has one dimension in one direction longer than a dimension in another direction. Such oblong holes in a tissue portion may be formed for example in tissue having a fiber direction, where the longest dimension of the hole may be aligned with the fiber direction.

In the embodiment illustrated in FIG. 42, the first surface 614 of the first portion MD′ is flat, thus providing a larger contact surface to the first tissue surface 616 and consequently less pressure on the tissue portion. A more stable position may also be achieved by the flat surface. Also, the second surface 620 of the second portion MD″ may be flat. However, other shapes, such as those described in other parts of the present disclosure, are possible.

As shown in FIG. 44, the connecting portion MD-2 may have an elongated cross-section in the third plane. It may be particularly advantageous if the connecting portion MD-2 has a longer length 644 than width 648, said length 644 extending in the same direction as a length direction of the second portion MD″, i.e. in the same direction as an elongation of the second portion MD″. Hereby, the elongation of the connecting portion MD-2 may run in the same direction as an elongation of the hole in the tissue portion.

With reference to FIG. 45, the rotational displacement of first cross-sectional distance of the first cross-sectional area and the first cross-sectional distance of the second cross-sectional area is shown, here at an angle of about 45 degrees. Accordingly, there is a rotational displacement, in the first, second and third planes, between a length direction 633 of the first portion MD′ and a length direction 631 of the second portion MD″. Other angles of rotational displacement are possible, such as 60, 75, 90, 105, 120, 135 degrees, etc.

One and the same device MD may be capable of assuming several different arrangements with regard to a rotational displacement of the first portion MD′ and second portion MD″. In particular, this is possible when the first portion MD′ and/or the second portion MD″ is configured to detachably connect to the interconnecting portion MD-2. For example, a connection mechanism between the first portion MD′ and the connecting portion MD-2, or between the second portion MD″ and the connecting portion MD-2, may possess a rotational symmetry to allow the first portion MD′ to be set in different positions in relation to the connecting portion MD-2 and in extension also in relation to the second portion MD″. Likewise, such rotational symmetry may allow the second portion MD-2″ to be set in different positions in relation to the connecting portion MD-2 and in extension also in relation to the first portion MD″.

With reference to FIGS. 46A to 46C, a procedure of insertion of the medical device MD in a tissue portion 610 will be described. The medical device MD may be oriented such that a length direction 631 of the second portion MD″ points downwards into the hole 611. Preferably, the second portion MD″ is positioned such that it is inserted close to an edge of the hole 611. The second portion MD″ may then be inserted partially through the hole 611 until the point where the first portion MD′ abuts the first tissue surface 616. Here, a 90 degrees rotational displacement between the first portion MD′ and the second portion MD″, as described above, will allow a relatively large portion of the second portion MD″ to be inserted before the first portion MD′ abuts the first tissue surface 616. Subsequently, the medical device MD may be pivoted to slide or insert the remaining portion of the second portion MD″ through the hole 611. While inserting the remaining portion of the second portion MD″, the tissue may naturally flex and move to give way for the second portion MD″. Upon having fully inserted the second portion MD″ through the hole 611 such that the second portion MD″ is completely located on the other side of the tissue portion 610, the tissue may naturally flex back.

### Pop Rivet Ceramic Coils

With reference to FIG. 47, an embodiment of an implantable energized medical device MD, which may be referred to as a remote unit in other parts of the present disclosure, will be described.

The medical device MD is configured to be held in position by a tissue portion 610 of a patient. The medical device MD comprises a first portion MD′ configured to be placed on a first side 612 of the tissue portion 610, the first portion MD′ having a first cross-sectional area in a first plane P1 and comprising a first surface 614 configured to face and/or engage a first tissue surface of the first side 612 of the tissue portion 610. The medical device MD further comprises a second portion MD″ configured to be placed on a second side 618 of the tissue portion 610, the second side 618 opposing the first side 612, the second portion MD″ having a second cross-sectional area in a second plane and comprising a second surface 620 configured to engage a second tissue surface of the second side 618 of the tissue portion 610. The medical device MD further comprises a connecting portion MD-2 configured to be placed through a hole in the tissue portion 610 extending between the first and second sides 612, 618 of the tissue portion 610. Here, the connecting portion MD-2 has a third cross-sectional area in a third plane. The connecting portion MD-2 is configured to connect the first portion MD′ to the second portion MD″.

At least one of the first portion and the second portion comprises at least one coil embedded in a ceramic material, the at least one coil being configured for at least one of: receiving energy transmitted wirelessly, transmitting energy wirelessly, receiving wireless communication, and transmitting wireless communication. In the illustrated embodiment, the first portion MD′ comprises a first coil 658 and a second coil 660, and the second portion MD″ comprises a third coil 662. The coils are embedded in a ceramic material 664

As discussed in other part of the present disclosure, the first portion MD′ may comprise a first wireless energy receiver configured to receive energy transmitted wirelessly from an external wireless energy transmitter, and further the first portion MD′ may comprise a first wireless communication receiver. The first wireless energy receiver and the first wireless communication receiver may comprise the first coil 658. Accordingly, the first coil 658 may be configured to receive energy wirelessly and/or to receive communication wirelessly.

By the expression “the receiver/transmitter comprising the coil” it is to be understood that said coil may form part of the receiver/transmitter.

The first portion MD′ comprises a distal end 665 and a proximal end 666, here defined with respect to the connecting portion MD-2. In particular, the proximal end 665 is arranged closer to the connecting portion MD-2 and closer to the second portion MD″ when the medical device MD is assembled. In the illustrated embodiment, the first coil 658 is arranged at the distal end 665.

The first portion MD′ may comprise an internal wireless energy transmitter and further a first wireless communication transmitter. In some embodiments, the internal wireless energy transmitter and/or the first wireless communication transmitter comprise(s) the first coil 658. However, in some embodiments the internal wireless energy transmitter and/or the first wireless communication transmitter comprises the second coil 660. Here, the second coil 660 is arranged at the proximal end 665 of the first portion MD′. Such placement of the second coil 660 may provide that energy and/or communication signals transmitted by the second coil 660 will not be attenuated by internal components of the first portion MD′ when being transmitted to the second portion MD″.

In some embodiments, the first wireless energy receiver and the internal wireless energy transmitter comprise a single coil embedded in a ceramic material. Accordingly, a single coil may be configured for receiving energy wirelessly and for transmitting energy wirelessly. Similarly, the first wireless communication receiver and the first wireless communication transmitter may comprise a single coil embedded in a ceramic material. Even further, in some embodiments a single coil may be configured for receiving and transmitting energy wirelessly, and for receiving and transmitting communication signals wirelessly.

The coils discussed herein are preferably arranged in a plane extending substantially parallel to the tissue portion 610.

The second portion MD″ may comprise a second wireless energy receiver and/or a second wireless communication receiver. In some embodiments, the third coil 662 in the second portion MD″ comprises the second wireless energy receiver and/or the second wireless communication receiver.

The second portion MD″ comprises a distal end 668 and a proximal end 670, here defined with respect to the connecting portion MD-2. In particular, the proximal end 668 is arranged closer to the connecting portion MD-2 and closer to the first portion MD′ when the medical device MD is assembled. In the illustrated embodiment, the third coil 662 is arranged at the proximal end 668 of the second portion MD″. Such placement of the third coil 662 may provide that energy and/or communication signals received by the third coil 662 will not be attenuated by internal components of the second portion MD″ when being received from the first portion MD′.

The first portion MD′ may comprise a first controller 300a connected to the first coil 658, second coil 660 and/or third coil 662. The second portion MD″ may comprise a second controller 300b connected to the first coil 658, second coil 660 and/or third coil 662.

In the illustrated embodiment, the first portion MD′ comprises a first energy storage unit 304a connected to the first wireless energy receiver 308a, i.e. the first coil 658. The second portion comprises a second energy storage unit 304b connected to the second wireless energy receiver 308b, i.e. the third coil 662. Such an energy storage unit may be a solid-state battery, such as a thionyl chloride battery.

In some embodiments, the first coil 658 is configured to receive energy transmitted wirelessly by the external wireless energy transmitter and to store the received energy in the first energy storage unit 304a. Furthermore, the first coil 658 and/or the second coil 660 may be configured to wirelessly transmit energy stored in the first energy storage unit 304a to the third coil 662, and the third coil 662 may be configured to receive energy transmitted wirelessly by the first coil 658 and/or the second coil 660 and to store the received energy in the second energy storage unit 305b.

The first energy storage unit 304a may be configured to store less energy than the second energy storage unit 304b and/or to be charged faster than the second energy storage unit 304b. Herein, charging of the first energy storage unit 304a may be relatively quick, whereas transfer of energy from the first energy storage unit 304a to the second energy storage unit 304b may be relatively slow. Thus, a user can quickly charge the first energy storage unit 304a and will not-during such charging—be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b via the first and/or second coil and the third coil.

Pop rivet gear

FIGS. 48A and 48B illustrate a gear arrangement and magnetic coupling for coupling the implantable energized medical device MD to an implant exerting force on a body part, and in particular a gear arrangement for transferring mechanical movement through an outer housing of the medical device MD or an outer housing of the second portion MD″.

The housing 484 of the medical device MD or second portion MD″ may be present in some embodiments of the medical device MD. In such embodiments, the housing 484 is configured to enclose, at least, the controller (not shown), motor M, any receivers and transmitters if present (not shown), and any gear arrangements G, G1, G2 if present. Herein, such features are protected from bodily fluids. The housing 484 may be an enclosure made from one of or a combination of: a carbon-based material (such as graphite, silicon carbide, or a carbon fiber material), a boron material, a polymer material (such as silicone, Peek R, polyurethane, UHWPE or PTFE), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability such that migration of fluid through the walls of the enclosure is prevented.

The implantable energized medical device may comprise at least part of a magnetic coupling, such as a magnetic coupling part 490a. A complementary part of the magnetic coupling, such as magnetic coupling part 490b, may be arranged adjacent to the medical device MD, so as to magnetically couple to the magnetic coupling part 490a and form the magnetic coupling. The magnetic coupling part 490b may form part of an entity not forming part of the medical device MD. However, in some embodiments the second portion MD″ comprises several chambers being hermetically sealed from each other. Such chambers may be coupled via the magnetic coupling as discussed herein. The magnetic coupling 490a, 490b provides for that mechanical work output by the medical device MD via, e.g., an electric motor can be transferred from the medical device MD to, e.g., an implant configured to exert force on a body part of a patient. In other words, the magnetic coupling 490a, 490b provides for that mechanical force can be transferred through the housing 484.

The coupling between components, such as between a motor and gear arrangement, or between a gear arrangement and a magnetic coupling, may be achieved by, e.g., a shaft or the like.

In some embodiments, for example as illustrated in FIG. 48A, a force output by a motor M in the second portion MD″ is connected to the magnetic coupling part 490a. The magnetic coupling part 490a transfers the force output from the motor M to the magnetic coupling part 490b, i.e. via the magnetic coupling 490a, 490b. The force output transferred via the magnetic coupling 490a, 490b here has a torque T1, which is substantially the same torque as delivered by the motor M. The magnetic coupling part 490b is connected to a gear arrangement G, located external to the medical device MD, for example in a medical implant configured to exert force on a body part or intermediate to a medical implant configured to exert force on a body part. The gear arrangement G is configured to increase the torque of the force delivered via the magnetic coupling 490a, 490b to deliver a force with torque T2 being higher than torque T1 to a medical implant. Consequently, low torque may be provided by the motor M, i.e. a relatively small force with high angular velocity, which is transferred via the magnetic coupling 490a, 490b before the torque is increased via gear arrangement G to achieve a relatively large force with low angular velocity. Hereby, the magnetic coupling 490a, 490b may utilize relatively weak magnetic forces to transfer the mechanical work through the housing 484 of the medical device MD without the risk of slipping between the magnetic coupling parts 490a, 490b.

In some embodiments, for example as illustrated in FIG. 48A, a force output of a motor M in the second portion MD″ is connected to a first gear arrangement G1, which in turn is coupled to the magnetic coupling part 490a. The motor M here provides a mechanical force with torque TO. The magnetic coupling part 490a transfers the force output from the motor M to the first gear arrangement G1. The first gear arrangement G1 is configured to increase the torque of the force delivered from the motor M to deliver a force with a higher torque T1 to the magnetic coupling 490a, 490b. The magnetic coupling part 490a transfers the force with torque T1 to the magnetic coupling part 490b. The magnetic coupling part 490b is connected to a second gear arrangement G2 located external to the medical device, for example in a medical implant configured to exert force on a body part, or intermediate to a medical implant configured to exert force on a body part. The second gear arrangement G2 is configured to increase the torque of the force delivered via the magnetic coupling 490a, 490b to deliver a force with torque T2 being higher than torque T1, and thus higher than torque TO, to a medical implant. Consequently, low torque may be provided by the motor M, i.e. a relatively small force with high angular velocity. The torque of the force provided by the motor M is then increased by the first gear arrangement G1, before the force is transferred via the magnetic coupling 490a, 490b. The torque of the force transferred via the magnetic coupling 490a, 490b is then yet again increased via the second gear arrangement G2 to achieve a relatively large force with low angular velocity. Hereby, the magnetic coupling 490a, 490b may utilize relatively weak magnetic forces to transfer the mechanical work through the housing 484 of the medical device MD without the risk of slipping between the magnetic coupling parts 490a, 490b. Furthermore, since some of the torque increase is made within the second portion MD″, and a remaining portion of the torque increase is made external to the medical device and the second portion MD″, the gear arrangements G1. G2 may be sized and configured appropriately to share the work of increasing the torque.

### Pop Rivet Tapered

With reference to FIGS. 49A-C. 50, 51, 52 and 53A-C, embodiments of an energized medical device MD, which may be referred to as a remote unit in other parts of the present disclosure, will be described. As illustrated, these implantable energized medical devices have a second portion being shaped in a particular manner in order to facilitate removal of the implantable energized medical device once it has been implanted for a period of time and fibrotic tissue has begun to form around the second portion. It is hereby disclosed that these types of second portions, as illustrated in FIGS. 49A-C. 50, 51, 52 and 53A-C, and as disclosed below, may be combined with any of the other features of the implantable energized medical device discussed in the present disclosure.

The device MD is configured to be held in position by a tissue portion 610 of a patient. The device MD comprises a first portion MD′ configured to be placed on a first side 612 of the tissue portion 610, the first portion MD′ having a first cross-sectional area in a first plane and comprising a first surface configured to face and/or engage a first tissue surface 616 of the first side 612 of the tissue portion 610. The device MD further comprises a second portion MD″ configured to be placed on a second side 618 of the tissue portion 610, the second side 618 opposing the first side 612, the second portion MD″ having a second cross-sectional area in a second plane and comprising a second surface configured to engage a second tissue surface 622 of the second side 618 of the tissue portion 610. The device MD further comprises a connecting portion MD-2 configured to be placed through a hole in the tissue portion 610 extending between the first and second sides 612. 618 of the tissue portion 610. The connecting portion MD-2 here has a third cross-sectional area in a third plane. The connecting portion MD-2 is configured to connect the first portion MD′ to the second portion MD″. In the illustrated embodiment, a connecting interface 630 between the connecting portion MD-2 and the second portion MD″ is arranged at an end of the second portion MD″.

The first portion MD′ may have an elongated shape. Similarly, the second portion MD″ may have an elongated shape. However, the first portion MD′ and/or second portion MD″ may assume other shapes, such as a flat disk e.g. having a width and length being larger than the height, a sphere, an ellipsoid, or any other polyhedral or irregular shape, some of these being exemplified in FIGS. 37 to 39.

To provide a frame of reference for the following disclosure, and as illustrated in FIGS. 50, 51 and 52, a first direction 631 is here parallel to the line A-A, to the second plane, and to a length of the second portion MD″. A second direction 633 is here parallel to the line B-B, to the second plane, and to a width of the second portion MD″. The second portion MD″ has a first end 632 and a second end 634 opposing the first end 632. The length of the second portion MD″ is defined as the length between the first end 632 and the second end 634. The length of the second portion MD″ is furthermore extending in a direction being different to the central extension C1 of the connecting portion MD-2. The first end 632 and second end 634 are separated in a direction parallel to the second plane. Similarly, the first portion MD′ has a length between a first and a second end, the length extending in a direction being different to the central extension C1 of the connecting portion MD-2.

The first portion MD′, connecting portion MD-2 and second portion MD″ may structurally form one integral unit. It is however also possible that the first portion MD′ and the connecting portion MD-2 structurally form one integral unit, while the second portion MD″ form a separate unit, or, that the second portion MD″ and the connecting portion MD-2 structurally form one integral unit, while the first portion MD′ form a separate unit.

Additionally, or alternatively, the second portion MD″ may comprise a removable and/or interchangeable portion 639 as described in other parts of the present disclosure.

In the following paragraphs, some features and properties of the second portion MD″ will be described. It is however to be understood that these features and properties may also apply to the first portion MD′.

The second portion MD″ has an intermediate region 638, and a distal region 640. A proximal region may be present, as described in other parts of the present disclosure. The intermediate region 638 is defined by the connecting interface 630 between the connecting portion MD-2 and the second portion MD″, and the distal region 640 extends from the connecting interface 630 between the connecting portion MD-2 and the second portion MD″ to the second end 634.

The first surface 614 configured to face and/or engage the first tissue surface 616 of the first side 612 of the tissue portion 610 may be substantially flat. In other words, the first portion MD′ may comprise a substantially flat side facing towards the tissue portion 610. Furthermore, an opposing surface of the first portion MD′, facing away from the tissue portion 610, may be substantially flat. Similarly, the second surface 620 configured to engage the second tissue surface 622 of the second side 618 of the tissue portion 610 may be substantially flat. In other words, the second portion MD″ may comprise a substantially flat side facing towards the tissue portion 610. Furthermore, an opposing surface of the second portion MD″, facing away from the tissue portion 610, may be substantially flat.

The second portion MD″ may be tapered from the first end 632 to the second end 634, thus giving the second portion MD″ different heights and/or widths along the length of the second portion MD″. The second portion may also be tapered from each of the first end 632 and second end 634 towards the intermediate region 638 of the second portion MD″.

Still referring to FIGS. 49A-C, 50, 51, 52, and 53A-C, the second portion MD″ and connecting portion MD-2 here form a connecting interface 630. Furthermore, the second portion MD″ has a lengthwise cross-sectional area along the first direction, wherein a second lengthwise cross-sectional area 690 is smaller than a first lengthwise cross-sectional area 689 and wherein the first lengthwise cross-sectional area 689 is located closer to the connecting interface 630 with regard to the first direction 631. Hereby, a tapered second portion is formed, being tapered towards the second end 634. The lengthwise cross-sectional area of the second portion MD″ may decrease continuously from an end of the intermediate region 638 towards the second end 634, as illustrated for example in FIG. 50. The decrease may be linear, as illustrated for example in FIG. 50. However, other types of decreasing lengthwise cross-sectional areas are possible, such as a parabolic, exponential, stepwise, or stepwise with radiused edges between each step thus forming a smooth rounded contour.

FIGS. 49B and 49C illustrate how the lengthwise cross-sectional area decrease over the length of the second portion MD″ towards the second 634, as viewed along the line A-A. FIG. 49B illustrate the first lengthwise cross-sectional area 689, and FIG. 49C illustrate the second lengthwise cross-sectional area 690.

In some embodiments, the lengthwise cross-sectional area may decrease over a majority of the length of the second portion towards the second end 634. In some embodiments, a decrease of the lengthwise cross-sectional area over at least ¼ of the length of the second portion towards the second end 634 may be sufficient. In the example illustrated in FIG. 50, the lengthwise cross-sectional area decrease over about 85% of the length of the second portion.

With the second portion MD″ having rotational symmetry along the first direction 631, as illustrated for example in FIG. 49A, the shape of the second portion MD″ may be conical.

As illustrated in FIG. 51, the second portion MD″ may have an upper surface, which include the second surface 620 configured to engage a second tissue surface of the second side of the tissue portion as discussed in other parts of the present disclosure, wherein the upper surface or second surface 620 is substantially flat and parallel to the second plane. In some embodiments the upper surface may be substantially perpendicular to the central extension C1 of the connecting portion MD-2. Hereby, the second surface may be configured to lay flat against the second side of the tissue portion. In such embodiments, a lower surface of the second portion MD″, opposite the second surface 620 and facing away from the first portion MD″, may be configured to taper towards the second end 634, thus achieving the decreasing lengthwise cross-sectional area along the first direction 631 towards the second end 634.

FIG. 52 illustrate an embodiment wherein the lengthwise cross-sectional area decrease in a stepwise manner towards the second end 634 of the second portion MD″. Here, the second portion MD″ has three major segments 692, 693, 694 having substantially constant diameter and each respective diameter being smaller moving towards the second end 634, being connected by intermediate segments 695, 696, wherein the diameter decreases along the first direction 631. Other variations of major segments having substantially constant diameter, and intermediate segments, having a decreasing diameter along the first direction 632, are possible, such as at least two major segments connected by a single intermediate segment with decreasing diameter, at least four major segments connected by three intermediate segments with decreasing diameter, and so on.

Referring now to FIGS. 53A-C, an implantable energized medical device similar to the one illustrated in FIG. 51 is illustrated. As can be seen in the perspective view of FIG. 53A, the second portion MD″ has a decreasing lengthwise cross-sectional area towards the second end. The upper surface 697 is also visible in this view, being substantially flat and providing a contact area to the second tissue surface 622. The first lengthwise cross-sectional area 689 is larger than the second cross-sectional area 690, as can be seen in FIGS. 53B and 53C, and the first lengthwise cross-sectional area 689 is located closer to the connecting interface between the connecting portion MD-2 and the second portion MD″ with regard to the first direction.

Further Aspect Combinable with any One of the Other Aspects-Communication

### General Communication Housing

1. An external device configured for communication with an implantable medical device implanted in a patient, the external device comprising:
   a display device,
   a housing unit configured to mechanically and disconnectably connect to the display device, the housing unit comprising:
   a first communication unit for receiving communication from the display device, and
   a second communication unit for wirelessly transmitting communication to the implantable medical device.
2. The external device according to aspect 1, wherein the external device comprises a handheld electronic device.
3. The external device according to any one of aspects 1 and 2, wherein the external device is configured for communicating with the implantable medical device for changing an operational state of the implantable medical device.
4. The external device according to any one of the preceding aspects, wherein the first communication unit is a wireless communication unit for wireless communication with the display device.
5. The external device according to aspect 4, wherein:
   the first communication unit is configured to communicate wirelessly with the display device using a first communication frequency,
   the second communication unit is configured to communicate wirelessly with the implantable medical device using a second communication frequency, and
   the first and second communication frequencies are different.
6. The external device according to any one of the preceding aspects, wherein the second communication unit is configured to communicate wirelessly with the implantable medical device using electromagnetic waves at a frequency below 100 KHz.
7. The external device according to any one of the preceding aspects, wherein the second communication unit is configured to communicate wirelessly with the implantable medical device using electromagnetic waves at a frequency below 40 KHz.
8. The external device according to any one of aspects 4-7, wherein the first communication unit is configured to communicate wirelessly with the display device using electromagnetic waves at a frequency above 100 kHz.
9. The external device according to any one of the preceding aspects, wherein:
   the first communication unit is configured to communicate with the display device using a first communication protocol,
   the second communication unit is configured to communicate wirelessly with the implantable medical device using a second communication protocol, and
   the first and second communication protocols are different.
10. The external device according to any one of aspects 3-9, wherein the housing unit comprises:
   a first antenna configured for wireless communication with the display device, and
   a second antenna configured for wireless communication with the implantable medical device.
11. The external device according to any one of aspects 1-3, wherein the first communication unit is a wire-based communication unit for wire-based communication with the display device.
12. The external device according to any one of the preceding aspects, wherein the display device comprises:
   a first communication unit for communication with the housing unit, and
   a second communication unit for wireless communication with a second external device.
13. The external device according to aspect 12, wherein the second communication unit of the display device is configured for communicating with the second external device over the Internet
14. The external device according to any one of aspects 12 and 13, wherein the first communication unit of the display device is a wireless communication unit for wireless communication with the housing unit.
15. The external device according to aspect 14, wherein:
   the first communication unit of the display device is configured to communicate wirelessly with the housing unit using a first communication frequency,
   the second communication unit of the display device is configured to communicate wirelessly with the second external device using a second communication frequency, and
   the first and second communication frequencies are different.
16. The external device according to any one of aspects 14 and 15, wherein:
   the first communication unit of the display device is configured to communicate wirelessly with the housing unit using a first communication protocol,
   the second communication unit of the display device is configured to communicate wirelessly with the second external device using a second communication protocol, and
   the first and second communication protocols are different.
17. The external device according to any one of aspects 14-16, wherein the display device comprises:
   a first antenna configured for wireless communication with the housing, and
   a second antenna configured for wireless communication with the second external device.
18. The external device according to any one of aspects 12-13, wherein the first communication unit is a wire-based communication unit for wire-based communication with the housing unit.
19. The external device according to any one of the preceding aspects, wherein the display device is configured to display a user interface to the patient.
20. The external device according to any one of the preceding aspects, wherein the housing unit is configured to transmit information pertaining to the display of the user interface to the display device.
21. The external device according to any one of aspects 19 and 20, wherein the display device is configured to:
   receive input pertaining to communication to or from the implantable medical device from the patient, and
   transmit communication based on the received input to the housing unit.
22. The external device according to any one of aspects 19-21, wherein the display device comprises a touch screen configured to display the user interface and receive the input from the patient.
23. The external device according to any one of the preceding aspects, wherein the housing unit is configured to display a user interface to the patient.
24. The external device according to any one of the preceding aspects, wherein the first communication unit of the housing unit is configured to receive communication from the implantable medical device pertaining to input from the patient, and wirelessly transmit communication based on the received input to the implantable medical device, using the second communication unit.
25. The external device according to any one of the preceding aspects, wherein the second communication unit of the housing unit is configured for wireless communication with the implantable medical device using a standard network protocol.
26. The external device according to aspect 25, wherein the standard network protocol is selected from a list comprising:
   RFID-type protocol,
   WLAN-type protocol,
   Bluetooth-type protocol,
   BLE-type protocol,
   NFC-type protocol,
   3G/4G/5G-type protocol, and
   GSM-type protocol.
27. The external device according to aspect 25, wherein the second communication unit of the housing unit comprises a Bluetooth transceiver.
27. The external device according to any one of the preceding aspects, wherein the second communication unit of the housing unit is configured for wireless communication with the implantable medical device using a proprietary network protocol.
28. The external device according to any one of aspects 25-27, wherein the second communication unit of the housing unit comprises a UWB transceiver.
29. The external device according to any one of aspects 4-28, wherein the first communication unit of the housing unit is configured for wireless communication with the display device using a standard network protocol.
30. The external device according to aspect 29, wherein the standard network protocol is an NFC type protocol.
31. The external device according to any one of aspects 4-28, wherein the first communication unit of the housing unit is configured for wireless communication with the display device using a proprietary network protocol.
32. The external device according to any one of aspects 4-31, wherein a communication range of the first communication unit of the housing unit is less than a communication range of the second communication unit of the housing unit.
33. The external device according to any one of aspects 14-32, wherein a communication range of the first communication unit of the display device is less than a communication range of the second communication unit of the display device.
34. The external device according to any one of the preceding aspects, wherein at least one of the housing unit and the display device is configured allow communication between the housing unit and the display device on the basis of a distance between the housing unit and the display device.
35. The external device according to any one of the preceding aspects, wherein at least one of the housing unit and the display device is configured allow communication between the housing unit and the display device on the basis of the housing unit being mechanically connected to the display device.
36. The external device according to any one of the preceding aspects, wherein the housing unit is configured allow communication between the housing unit and the implantable medical device on the basis of a distance between the housing unit and the implantable medical device.
37. The external device according to any one of the preceding aspects, wherein the housing unit further comprises an encryption unit configured to encrypt communication received from the display device.
38. The external device according to aspect 37, wherein the housing unit is further adapted to transmit the encrypted communication, using the second communication unit, to the implantable medical device.
39. The external device according to any one of aspects 14-38, wherein the second communication unit of the display device is configured to be disabled to enable at least one of:
   communication between the display device and the housing unit, and communication between the housing unit and the implantable medical device.
40. The external device according to any one of the preceding aspects, wherein the display device is a wearable device or a handset.
41. The external device according to aspect 40, wherein the housing unit comprises a case for the wearable device or handset.
42. The external device according to any one of the preceding aspects, wherein the implantable medical device is an implantable medical device configured to exert a force on a body portion of the patient.
43. The external device according to any one of the preceding aspects, wherein the implantable medical device comprises an electrical motor and a controller (300) for controlling the electrical motor.
44. The external device according to any one of aspects 1-41, wherein the implantable medical device comprises an energy consuming part.
45. A housing unit configured for communication with an implantable medical device when implanted in a patient, the housing unit being configured to mechanically and disconnectably connect to a display device and comprising
   a first communication unit for receiving communication from the display device and
   second communication unit for wirelessly transmitting communication to the implantable medical device.
46. The housing unit according to aspect 45, wherein the display device is a wearable device or a handset and the housing unit comprises a case for the wearable device or handset.
47. The housing unit according to any one of aspects 45-46, wherein the first communication unit is a wireless communication unit for wireless communication with the display device.
48. The housing unit according to aspect 47, wherein:
   the first communication unit is configured to communicate wirelessly with the display device using a first communication frequency,
   the second communication unit is configured to communicate wirelessly with the implantable medical device using a second communication frequency, and
   the first and second communication frequencies are different.
49. The housing unit according to any one of aspects 45-48, wherein the second communication unit is configured to communicate wirelessly with the implantable medical device using electromagnetic waves at a frequency below 100 KHz.
50. The housing unit according to any one of aspects 45-49, wherein the second communication unit is configured to communicate wirelessly with the implantable medical device using electromagnetic waves at a frequency below 40 KHz.
51. The housing unit according to any one of aspects 47-50, wherein the first communication unit is configured to communicate wirelessly with the display device using electromagnetic waves at a frequency above 100 kHz.
52. The housing unit according to any one of aspects 45-51, wherein:
   the first communication unit is configured to communicate wirelessly with the display device using a first communication protocol,
   the second communication unit is configured to communicate wirelessly with the implantable medical device using a second communication protocol, and
   the first and second communication protocols are different.
53. The housing unit according to any one of aspects 47-52, wherein the housing unit comprises:
   a first antenna configured for wireless communication with the display device, and
   a second antenna configured for wireless communication with the implantable medical device.
54. The housing unit according to any one of aspects 45-46, wherein the first communication unit is a wire-based communication unit for wire-based communication with the display device.
55. The housing unit according to any one of aspects 45-54, wherein the housing unit is configured to transmit information pertaining to the display of a user interface to the display device.
56. The housing unit according to any one of aspects 45-55, wherein the housing unit is configured to receive patient input from the display device.
57. The housing unit according to any one of aspects 45-56, wherein the housing unit is configured to display a user interface to the patient.
58. The housing unit according to any one of aspects 45-57, wherein the second communication unit is configured for wireless communication with the implantable medical device using a standard network protocol.
59. The housing unit according to aspect 58, wherein the standard network protocol is one selected
   from a list comprising:
   RFID-type protocol,
   WLAN-type protocol,
   Bluetooth-type protocol,
   BLE-type protocol,
   NFC-type protocol,
   3G/4G/5G-type protocol, and
   GSM-type protocol.
60. The housing unit according to aspect 58, wherein the second communication unit comprises a Bluetooth transceiver.
61. The housing unit according to any one of aspects 45-57, wherein the second communication unit is configured for wireless communication with the implantable medical device using a proprietary network protocol.
62. The housing unit according to any one of aspects 58-61, wherein the second communication unit of the housing unit comprises a UWB transceiver.
63. The housing unit according to any one of aspects 47-62, wherein the first communication unit of the housing unit is configured for wireless communication with the display device using a standard network protocol.
64. The housing unit according to aspect 63, wherein the standard network protocol is an NFC type protocol.
65. The housing unit according to any one of aspects 47-62, wherein the first communication unit of the housing unit is configured for wireless communication with the display device using a proprietary network protocol.
66. The housing unit according to any one of aspects 47-65, wherein a communication range of the first communication unit is less than a communication range of the second communication unit.
67. The housing unit according to any one of aspects 45-66, wherein the housing unit is configured allow communication between the housing unit and the display device on the basis of a distance between the housing unit and the display device.
68. The housing unit according to any one of aspects 45-67, wherein the housing unit is configured allow communication between the housing unit and the display device on the basis of the housing unit being mechanically connected to the display device.
69. The housing unit according to any one of aspects 45-68, wherein the housing unit is configured allow communication between the housing unit and the implantable medical device on the basis of a distance between the housing unit and the implantable medical device.
70. The housing unit according to any one of aspects 45-69, wherein the housing unit further comprises an encryption unit configured to encrypt communication received from the display device.
71. The housing unit according to aspect 70, wherein the housing unit is further adapted to transmit the encrypted communication, using the second communication unit, to the implantable medical device.
72. The housing unit according to aspects 45-71, wherein the minimum bounding box of the housing unit and the display device when mechanically connected, is no more than: 10% wider, 10% longer or 100% higher, than the minimum bounding box of the display device.
73. The housing unit according to aspects 45-72, wherein the housing unit comprises one or more switches configured to, when the housing is not mechanically connected to the display device, be used by the patient.
74. The housing unit according to aspect 73, wherein the switches are at least partly covered by the display device, when the display device is mechanically connected to the housing unit.
75. The housing unit according to any one of aspects 45-74, wherein at least a part of the housing unit is configured to bend to mechanically connect to the display device.
76. The housing unit according to any one of aspects 45-75, wherein at least a part of the housing unit is configured to covers at least one side of the display device.
77. The housing unit according to any one of aspects 45-76, wherein the housing unit is configured to clasp the display device.
78. The housing unit according to any one of aspects 45-76, wherein the housing unit is configured to mechanically connect to the display unit by an attachment device mechanically connected to the housing unit and to the display device.
79. The housing unit according to any one of aspects 45-76, wherein the housing unit comprises a magnet for magnetically attaching the housing unit to the display device.
80. The housing unit according to any one of aspects 45-79, wherein the housing unit is configured to communicate with an implantable medical device configured to exert a force on a body portion of the patient.
81. The external device according to any one of aspects 45-80, wherein the housing unit is configured to communicate with an implantable medical device comprising an electrical motor and a controller for controlling the electrical motor.

### General Security Mode

1. An implantable controller for an implantable medical device, the implantable controller comprises:
   a wireless transceiver for communicating wirelessly with an external device, a security module, and
   a central unit configured to be in communication with the wireless transceiver, the security module and the implantable medical device:
   the wireless transceiver is configured to receive communication from the external device including at least one instruction to the implantable medical device, and transmit the received communication to the central unit,
   the central unit is configured to send secure communication to the security module, derived from the received communication from the external device, and the security module is configured to at least one of:
   decrypt at least a portion of the secure communication, and
   verify the authenticity of the secure communication, and
   the security module is configured to transmit a response communication to the central unit, and
   the central unit is configured to communicate the at least one instruction to the implantable medical device, the at least one instruction being based on:
   the response communication, or
   a combination of the response communication and the received communication from the external device.
2. The implantable controller according to aspect 1, wherein the security module comprises a set of rules for accepting communication from the central unit.
3. The implantable controller according to aspect 2, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
4. The implantable controller according to aspect 4, wherein the set of rules comprises a rule stipulating that communication from the central unit is only accepted when the wireless transceiver has been placed in the off-mode for a specific time period.
5. The implantable controller according to any one of the preceding aspects wherein the central unit is configured to verify a digital signature of the received communication from the external device.
6. The implantable controller according to aspect 4, wherein the set of rules comprises a rule stipulating that communication from the central unit is only accepted when the digital signature of the received communication has been verified by the central unit.
7. The implantable controller according to any one of the preceding aspects, wherein the central unit is configured to verify the size of the received communication from the external device.
8. The implantable controller according to aspect 7, wherein the set of rules comprises a rule stipulating that communication from the central unit is only accepted when the size of the received communication has been verified by the central unit.
9. The implantable controller according to any one of the preceding aspects, wherein:
   the wireless transceiver is configured to receive a message from the external device being encrypted with at least a first and second layer of encryption,
   the central unit is configured to decrypt a first layer of decryption and transmit at least a portion of the message comprising the second layer of encryption to the security model, and
   the security module is configured to decrypt the second layer of encryption and transmit a response communication to the central unit based on the portion of the message decrypted by the security module.
10. The implantable controller according to aspect 9, wherein the central unit is configured to decrypt a portion of the message comprising a digital signature, such that the digital signature can be verified by the central unit.
11. The implantable controller according to aspect 9, wherein the central unit is configured to decrypt a portion of the message comprising message size information, such that the message size can be verified by the central unit.
12. The implantable controller according to aspect 9, wherein the central unit is configured to decrypt a first and second portion of the message, and wherein the first portion comprises a checksum for verifying the authenticity of the second portion.
13. The implantable controller according to any one of aspects 9-12, wherein the response communication transmitted from the security module comprises a checksum, and wherein the central unit is configured to verify the authenticity of at least a portion of the message decrypted by the central unit using the received checksum.
14. The implantable controller according to aspect 4, wherein the set of rules comprises a rule related to the rate of data transfer between the central unit and the security module.
15. The implantable controller according to any one of aspects 9-14, wherein the security module is configured to decrypt a portion of the message comprising a digital signature, encrypted with the second layer of encryption, such that the digital signature can be verified by the security module.
16. The implantable controller according to any one of aspects 4-15, wherein the central unit is only capable of decrypting a portion of the receive communication from the external device when the wireless transceiver is placed in the off-mode.
17. The implantable controller according to any one of aspects 4-16, wherein the central unit is only capable of communicating the at least one instruction to the implantable medical device when the wireless transceiver is placed in the off-mode.
18. The implantable controller according to any one of the preceding aspects, wherein the implantable controller is configured to:
   receive, using the wireless transceiver, a message from the external device comprising a first non-encrypted portion and a second encrypted portion,
   decrypt the encrypted portion, and
   use the decrypted portion to verify the authenticity of the non-encrypted portion.
19. The implantable controller according to aspect 18, wherein the central unit is configured to:
   transmit the encrypted portion to the security module,
   receive a response communication from the security module, based on information contained in the encrypted portion being decrypted by the security module,
   and use the response communication to verify the authenticity of the non-encrypted portion.
20. The implantable controller according to any one of aspects 18-19, wherein the non-encrypted portion comprises at least a portion of the at least one instruction to the implantable medical device.
21. The implantable controller according to any one of the preceding aspects, wherein the implantable controller is configured to:
   receive, using the wireless transceiver, a message from the external device comprising information related to at least one of: a physiological parameter of the patient and a physical parameter of the implanted medical device, and
   use the received information to verify the authenticity of the message.
22. The implantable controller according to aspect 21, wherein the physiological parameter of the patient comprises at least one of: a temperature, a heart rate and a saturation value.
23. The implantable controller according to aspect 21, wherein the physical parameter of the implanted medical device comprises at least one of: a current setting or value of the implanted medical device, a prior instruction sent to the implanted medical device or an ID of the implanted medical device.
24. The implantable controller according to any one of aspects 21-23, wherein the portion of the message comprising the information is encrypted, and wherein the central unit is configured to transmit the encrypted portion to the security module and receive a response communication from the security module, based on the information having been decrypted by the security module.
25. The implantable controller according to any one of the preceding aspects, wherein the security module comprises a hardware security module comprising at least one hardware-based key.
26. The implantable controller according to aspect 25, wherein the hardware-based key corresponds to a hardware-based key in the external device.
27. The implantable controller according to aspect 25, wherein the hardware-based key corresponds to a hardware-based key on a key-card connectable to the external device.
28. The implantable controller according to any one of the preceding aspects, wherein the security module comprises a software security module comprising at least one software-based key.
29. The implantable controller according to aspect 28, wherein the software-based key corresponds to a software-based key in the external device.
30. The implantable controller according to aspect 28, wherein the software-based key corresponds to a software-based key on a key-card connectable to the external device.
31. The implantable controller according to any one of the preceding aspects, wherein the security module comprises a combination of a software-based key and a hardware-based key.
32. The implantable controller according to any one of the preceding aspects, wherein the security module comprises at least one cryptoprocessor.
33. The implantable controller according to any one of the preceding aspects, wherein the wireless transceiver is configured to receive communication from a handheld external device.
34. The implantable controller according to any one of the preceding aspects, wherein the at least one instruction to the implantable medical device comprises an instruction for changing an operational state of the implantable medical device.
35. The implantable controller according to any one of the preceding aspects, wherein the wireless transceiver is configured to communicate wirelessly with the external device using electromagnetic waves at a frequency below 100 kHz.
36. The implantable controller according to aspect 35, wherein the wireless transceiver is configured to communicate wirelessly with the external device using electromagnetic waves at a frequency below 40 KHz.
37. The implantable controller according to any one of the preceding aspects, wherein:
   the wireless transceiver is configured to communicate wirelessly with the external device using a first communication protocol,
   the central unit is configured to communicate with the security module using a second communication protocol, and
   the first and second communication protocols are different.
38. The implantable controller according to any one of the preceding aspects, wherein the wireless transceiver is configured to communicate wirelessly with the external device using a standard network protocol.
39. The implantable controller according to aspect 38, wherein the standard network protocol is
   selected from a list comprising:
   RFID-type protocol,
   WLAN-type protocol,
   Bluetooth-type protocol,
   BLE-type protocol,
   NFC-type protocol,
   3G/4G/5G-type protocol, and
   GSM-type protocol.
40. The implantable controller according to any one of aspects 1-37, wherein the wireless transceiver is configured to communicate wirelessly with the external device using a proprietary network protocol.
41. The implantable controller according to any one of aspects 1-40, wherein the wireless transceiver comprises a UWB transceiver.
42. The external device according to any one of the preceding aspects, wherein the security module and the central unit are comprised in a controller.
43. The external device according to aspect 42, wherein the wireless transceiver is comprised in the controller.
44. The external device according to any one of the preceding aspects, wherein the implantable medical device is an implantable medical device configured to exert a force on a body portion of the patient.
45. The external device according to any one of the preceding aspects, wherein the implantable medical device comprises an electrical motor and wherein the controller is configured for controlling the electrical motor.

### Variable_Impedance_1

1. An implantable medical device comprising a receiving unit comprising:
   at least one coil configured for receiving transcutaneously transferred energy,
   a measurement unit configured to measure a parameter related to the energy received by the coil,
   a variable impedance electrically connected to the coil,
   a switch placed between the variable impedance and the coil for switching off the electrical connection between the variable impedance and the coil, and a controller configured to:
   control the variable impedance for varying the impedance and thereby tune the coil based on the measured parameter, and
   control the switch for switching off the electrical connection between the variable impedance and the coil in response to the measured parameter exceeding a threshold value.
2. The implantable medical device according to aspect 1, wherein the controller is configured to vary the variable impedance in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
6. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
7. The implantable medical device according to aspect 6, wherein the controller is configured to control the variable impedance in response to the pulse pattern deviating from a predefined pulse pattern.
8. The implantable medical device according to aspect 6, wherein the controller is configured to control the switch for switching off the electrical connection between the variable impedance and the coil in response to the pulse pattern deviating from a predefined pulse pattern.
9. The implantable medical device according to any one of the preceding aspects, wherein the measurement unit is configured to measure a temperature in the implantable medical device or in the body of the patient, and wherein the controller is configured to control the first and second switch in response to the measured temperature.
10. The implantable medical device according to any one of the preceding aspects, wherein the variable impedance comprises a resistor and a capacitor.
11. The implantable medical device according to any one of the preceding aspects, wherein the variable impedance comprises a resistor and an inductor.
12. The implantable medical device according to any one of the preceding aspects, wherein the variable impedance comprises an inductor and a capacitor.
13. The implantable medical device according to any one of the preceding aspects, wherein the variable impedance comprises a digitally tuned capacitor.
14. The implantable medical device according to any one of the preceding aspects, wherein the variable impedance comprises a digital potentiometer.
15. The implantable medical device according to any one of the preceding aspects, wherein the variable impedance comprises a variable inductor.
16. The implantable medical device according to any one of the preceding aspects, wherein the variation of the impedance is configured to lower the active power that is received by the receiving unit.
17. The implantable medical device according to any one of the preceding aspects, wherein the variable impedance is placed in series with the coil.
18. The implantable medical device according to any one of aspects 1-16, wherein the variable impedance is placed parallel to the coil.
19. The implantable medical device according to any one of the preceding aspects, further comprising an energy storage unit connected to the receiving unit, and wherein the energy storage unit is configured for storing energy received by the receiving unit.
20. The implantable medical device according to any one of the preceding aspects, further comprising an energy consuming part.
21. The implantable medical device according to aspect 20, wherein the energy consuming part of the implantable medical device is configured to exert a force on a body portion of the patient.
22. The implantable medical device according to aspect 20, wherein the energy consuming part of the implantable medical device comprises an electrical motor and wherein the controller is configured for controlling the electrical motor.

### Variable_Impedance_2

1. An implantable medical device comprising a receiving unit comprising:
   at least one coil configured for receiving transcutaneously transferred energy, a measurement unit configured to measure a parameter related to the energy received by the coil,
   a first switch is placed at a first end portion of the coil,
   a second switch placed at a second end portion of the coil, such that the coil can be completely disconnected from other portions of the implantable medical device, and
   a controller configured to control the first and second switch for completely disconnecting the coil from other portions of the implantable medical device on the basis of the measured parameter.
2. The implantable medical device according to aspect 1, wherein the controller is configured to control the first and second switch in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
6. The implantable medical device according to aspect 5, wherein the controller is configured to control the first and second switch in response to the pulse pattern deviating from a predefined pulse pattern.
7. The implantable medical device according to any one of the preceding aspects, wherein the measurement unit is configured to measure a temperature in the implantable medical device or in the body of the patient, and wherein the controller is configured to control the first and second switch in response to the measured temperature.
8. The implantable medical device according to any one of the preceding aspects, further comprising an energy storage unit connected to the receiving unit, and wherein the energy storage unit is configured for storing energy received by the receiving unit.
9. The implantable medical device according to any one of the preceding aspects, further comprising an energy consuming part.
10. The implantable medical device according to aspect 9, wherein the energy consuming part of the implantable medical device is configured to exert a force on a body portion of the patient.
11. The implantable medical device according to aspect 9, wherein the energy consuming part of the implantable medical device comprises an electrical motor and wherein the controller is configured for controlling the electrical motor.

### Variable_Impedance_3

1. An implantable medical device comprising a receiving unit comprising:
   at least one coil configured for receiving transcutaneously transferred energy, a measurement unit configured to measure a parameter related to the energy received by the coil, and
   a controller, wherein:
   the receiving unit is configured to receive transcutaneously transferred energy in pulses according to a pulse pattern, and
   the measurement unit is configured to measure a parameter related to the pulse pattern, and
   the controller is configured to control the receiving unit in response to the pulse pattern of the received energy deviating from a predetermined pulse pattern.
2. The implantable medical device according to aspect 1, further comprising at least one switch placed in series with the coil for switching of the coil, wherein the controller is configured to control the switch to switch of the coil in response to the pulse pattern of the received energy deviating from a predetermined pulse pattern.
3. The implantable medical device according to aspect 1, further comprising a variable impedance electrically connected to the coil, for varying the impedance and thereby tuning the coil, and wherein the controller is configured to control the variable impedance in response to the pulse pattern of the received energy deviating from a predetermined pulse pattern.
4. The implantable medical device according to any one of the preceding aspects, wherein the measurement unit is configured to measure a parameter related to the energy received by the coil over a time period.
5. The implantable medical device according to any one of the preceding aspects, wherein the measurement unit is configured to measure a parameter related to a change in energy received by the coil.
6. The implantable medical device according to any one of the preceding aspects, wherein the measurement unit is configured to measure a temperature in the implantable medical device or in the body of the patient, and wherein the controller is configured to control the first and second switch in response to the measured temperature.
7. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
8. The implantable medical device according to aspect 3, wherein the variable impedance comprises a resistor and a capacitor.
9. The implantable medical device according to aspect 3, wherein the variable impedance comprises a resistor and an inductor.
10. The implantable medical device according to aspect 3, wherein the variable impedance comprises an inductor and a capacitor.
11. The implantable medical device according to aspect 3, wherein the variable impedance comprises a digitally tuned capacitor.
12. The implantable medical device according to aspect 3, wherein the variable impedance comprises a digital potentiometer.
13. The implantable medical device according to aspect 3, wherein the variable impedance comprises a variable inductor.
14. The implantable medical device according to any one of aspects 3-12, wherein the variation of the impedance is configured to lower the active power that is received by the receiving unit.
15. The implantable medical device according to any one of aspects 3-13, wherein the variable impedance is placed in series with the coil.
16. The implantable medical device according to any one of aspects 3-13, wherein the variable impedance is placed parallel to the coil.
17. The implantable medical device according to any one of the preceding aspects, further comprising an energy storage unit connected to the receiving unit, and wherein the energy storage unit is configured for storing energy received by the receiving unit.
18. The implantable medical device according to any one of the preceding aspects, further comprising an energy consuming part.
19. The implantable medical device according to aspect 18, wherein the energy consuming part of the implantable medical device is configured to exert a force on a body portion of the patient.
20. The implantable medical device according to aspect 18, wherein the energy consuming part of the implantable medical device comprises an electrical motor and wherein the controller is configured for controlling the electrical motor.

### Method of Communication

1. A method of using the system for injecting a substance into a patient's body according to any one of the preceding aspects, comprising a step of wireless communication between components of the system.
2. The method according to aspect 1, comprising at least one of the following steps:
   encrypting the wireless communication from or to, or both from and to, a controller of the system,
   signing data transmitted by a controller via the wireless communication, and
   inputting authentication data of the patient to authenticate a user of the system.
3. The method according to aspect 2, wherein the step of encrypting the wireless communication includes encryption with a public key and decryption with a private key.
4. The method according to aspect 3, comprising the step of deriving the private key as a combined key by combining at least a first key and a second key.
5. The method according to any one of aspects 2 to 4, wherein the step of signing the data transmitted by the controller via the wireless communication involves use of a private key, wherein the method comprises the further step of verifying the signed data using a public key.
6 The method according to any one of aspects 2 to 5, comprising the step of obtaining authentication data of the patient.
7. The method according to aspect 6, wherein the step of obtaining authentication data of the patient includes obtaining such data using at least one of a fingerprint reader, a retina scanner, a camera, a graphical user interface for inputting a code, and a microphone.
8. The method according to any one of aspects 2 to 7, comprising the step of generating a sensation detectable by a sense of the patient and the step of authenticating a communication channel between two controllers of the system by inputting authentication data of the patient relating to the sensation.
9. The method according to aspect 8, wherein the step of authenticating the communication channel involves a step of verifying that the authentication data match data from a sensation generator relating to the sensation generated by the sensation generator.
10. The method according to aspect 8 or 9, wherein the step of generating a sensation detectable by the sense of the patient comprises generation of at least one of:
   a vibration, which includes or does not include a fixed-frequency mechanical vibration,
   a sound, which includes or does not include a superposition of fixed-frequency mechanical vibrations,
   a photonic signal, which includes or does not include a non-visible light pulse, such as an infrared pulse,
   a light signal, which includes or does not include a visual light pulse,
   an electrical signal, which includes or does not include an electrical current pulse, and
   a heat signal, which includes or does not include a thermal pulse.

1-135. (canceled)

136. A system for treating a patient having a disorder related to a patient's intestine, comprising a plurality of electrical stimulation devices, wherein each of the electrical stimulation devices comprises one or more electrodes for electrically stimulating muscle or neural tissue of the intestine and a wireless energy receiver configured to receive energy for stimulating the muscle or neural tissue wirelessly.

137. The system of claim 136, comprising an individual wireless energy transmitter for each one of the electrical stimulation devices and configured to transfer energy to the respective one of the electrical stimulation devices.

138. The system of claim 136, wherein the wireless energy receiver is configured to receive the energy via RFID pulses.

139. The system of claim 136, wherein each of the electrical stimulation devices comprises an internal controller.

140. The system of claim 139, wherein the internal controller includes an individual code by which it is individually addressable by an external controller or remote controller.

141. The system of claim 136, configured to electrically stimulate, by means of the electrodes of the electrical stimulation devices, the muscle or neural tissue sufficiently for a muscle of the intestine to contract to an extent such that the intestine constricts.

142. The system of claim 136, wherein the system is configured to be applied to a reservoir section of the intestine which is formed from surgically modified intestine that has been cut along a mutual contact line of laterally adjacent sections of a bent portion of intestine and connected so that the upper and lower halves of the cut intestine form an intestinal wall of the reservoir section.

143. The system of claim 136, wherein at least the electrodes of the electrical stimulation devices are configured to be implanted in surgically created folds of the patient's intestine.

144. The system of claim 136, comprising at least one mechanical or hydraulic constriction device configured to be implanted outside the patient's intestine in close proximity thereto for constricting the intestine from the outside thereof.

145. The system of claim 136, configured to electrically stimulate, by means of the electrodes of the electrical stimulation devices, the muscle or neural tissue in an area of the intestine constricted by the at least one mechanical or hydraulic constriction device sufficiently for increasing blood flow through the tissue of the intestine.

146. The system of claim 136, wherein the system is configured such that at least one of:
wireless communication from or to, or both from and to, a controller of the system is encrypted,
data transmitted by a controller via wireless communication is signed, and
authentication of a user of the system involves input of authentication data of the patient.

147. The system of claim 136, wherein the one or more electrodes comprise a bare electrode portion configured to form a metal-tissue interface with the tissue, thereby allowing faradaic charge transfer to be the predominant charge transfer mechanism over said interface.

148. The system of claim 136, wherein the one or more electrodes comprise an electrode portion at least partly covered by a dielectric material configured to form a dielectric-tissue interface with tissue, thereby allowing for a faradaic portion of the charge transfer mechanism over said interface to be reduced.

149. The system of claim 136, wherein the electrical stimulation devices are configured such that at least two of the electrodes can be arranged on opposing sides of the patient's intestine.

150. The system of claim 136, configured to control the electrodes such that the tissue is stimulated by a series of electrical pulses.

151. The system of claim 136, comprising an outer surface and a coating arranged on the outer surface.

152. The system of claim 136, wherein the system makes part of:
(a) an artificial sphincter which is configured, when implanted, to act on a wall of an intestine of a patient so as to restrict flow of intestinal contents through the intestine or
(b) an emptying device which is configured, when implanted, to act on a wall of an intestine of a patient so as to advance intestinal contents contained in the intestine out of the intestine.

153. A method of using a system for treating a patient having a disorder related to a patient's intestine, the system comprising a plurality of electrical stimulation devices, wherein each of the electrical stimulation devices comprises one or more electrodes for electrically stimulating muscle or neural tissue of the intestine and a wireless energy receiver configured to receive energy for stimulating the muscle or neural tissue wirelessly, wherein the method comprises at least one of the following steps:
(a) wirelessly transmitting energy to and receiving the energy by the energy receiver and
(b) electrically stimulating, by means of the electrodes of the plurality of electrical stimulation devices, the muscle tissue in an area of the intestine constricted by the at least one mechanical or hydraulic constriction device sufficiently for increasing blood flow through the tissue of the intestine.

154. A method of implanting a system for treating a patient having a disorder related to the patient's intestine, comprising the steps of:
making an incision in the body of the patient for accessing the intestine,
inserting a plurality of electrical stimulation devices, wherein each of the plurality of electrical stimulation devices comprises one or more electrodes for electrically stimulating muscle or neural tissue of the intestine and a wireless energy receiver configured to receive energy for stimulating the muscle or neural tissue wirelessly,
placing the electrodes of the plurality of electrical stimulation devices in connection with the intestine,
inserting one or a plurality of wireless energy transmitters,
placing the one or plurality of wireless energy transmitters in proximity to the plurality of electrical stimulation devices so as to allow transfer of energy from the one or plurality of energy transmitters to all of the plurality of electrical stimulation devices.
